(19) United States
(12) Patent Application Publication
(10) Pub. No.: US 2023/0405086 A1
(43) Pub. Date: Dec. 21, 2023

(54) COMPOSITIONS AND METHODS FOR PREVENTING, DETECTING, AND TREATING INFLAMMATORY BOWEL DISEASE

(71) Applicants: ICAHN SCHOOL OF MEDICINE AT MOUNT SINAI, New York, NY (US); THE GOVERNING COUNCIL OF THE UNIVERSITY OF TORONTO, Toronto (CA)

(72) Inventors: Sacha GNJATIC, New York, NY (US); Arthur MORTHA, Toronto (CA); Jean-Frederic COLOMBEL, New York, NY (US); Romain REMARK, New York, NY (US); Miriam MERAD, New York, NY (US)

(73) Assignees: ICAHN SCHOOL OF MEDICINE AT MOUNT SINAI, New York, NY (US); THE GOVERNING COUNCIL OF THE UNIVERSITY OF TORONTO, Toronto, ON (CA)

(21) Appl. No.: 17/997,616

(22) PCT Filed: Apr. 30, 2021
(86) PCT No.: PCT/US2021/030264

§ 371 Date: Oct. 31, 2022

Related U.S. Application Data
(60) Provisional application No. 63/018,960, filed on May 1, 2020.

(51) Int. Cl. A61K 38/19; G01N 33/564; A61P 1/12

(52) U.S. Cl. CPC A61K 38/193; G01N 33/564; A61P 1/12; G01N 2800/065; G01N 2333/535

(57) ABSTRACT

The present disclosure relates to a composition comprising a post-translationally modified Granulocyte Macrophage-Colony Stimulating Factor (GM-CSF) protein. The disclosure further relates to methods of preventing or treating Crohn's Disease and/or a condition resulting from Crohn's Disease in a subject. The disclosure further relates to methods for diagnosing and/or predicting severity of and/or treating Crohn's Disease in a subject. Also disclosed are methods for diagnosing inflammatory bowel disease in a subject and methods for diagnosing a pre-disease state of Crohn's Disease in a subject.

This application claims benefit of U.S. Provisional Patent Application Ser. No. 63/018,960, filed May 1, 2020, which is hereby incorporated by reference in its entirety.

### FIELD

The present disclosure relates generally to compositions and methods for preventing, detecting, and treating inflammatory bowel disease.

### BACKGROUND

Inflammatory bowel disease (IBD) is an increasingly diagnosed chronic inflammatory pathology of the gastrointestinal tract, affecting 0.3% of the world's population. Ng et al., “Worldwide Incidence and Prevalence of Inflammatory Bowel Disease in the 21st Century: A Systematic Review of Population-Based Studies,” Lancet 390:2769-2778 (2018). IBD is broadly sub-classified into Ulcerative Colitis (UC) and Crohn's Disease (CD) based on general pathological appearance. While both pathologies affect the gastrointestinal tract, what causes them remains puzzling and renders both diseases multifactorial. O'Toole and Korzenik, “Environmental triggers for IBD,” Current Gastroenterology Reports 16:396 (2014) and Ananthakrishnan et al., “Environmental Triggers in IBD: a Review of Progress and Evidence,” Nature Reviews. Gastroenterology and Hepatology 15:39-49 (2018). Genome-wide association studies (GWAS) provide strong support that IBD is a pathology driven by mono and multigenetic variations (McGovern et al., “Genetics of Inflammatory Bowel Diseases,” Gastroenterology 149: 1163-1176 (2015) and Plevy et al., “Combined Serological, Genetic, and Inflammatory Markers Differentiate non-IBD, Crohn's Disease, and Ulcerative Colitis Patients,” Inflammatory Bowel Disease 19:1139-1148 (2013)), but non-genetic and environmental factors are increasingly considered as contributors to the heterogeneity of this disease. Silverberg et al., “Toward an Integrated Clinical, Molecular and Serological Classification of Inflammatory Bowel Disease: Report of a Working Party of the 2005 Montreal World Congress of Gastroenterology,” Canadian Journal of Gastroenterology 19 Suppl A:5A-36A (2005). Finding factors contributing to IBD development that may predict disease onset is therefore of high clinical relevance.

A key immunologic characteristic of IBD is the break in intestinal homeostasis, commonly manifested through insufficient barrier integrity, decreased immunologic tolerance, or excessive inflammation. Abraham and Cho, “Inflammatory Bowel Disease,” The New England Journal of Medicine 361:2066-2078 (2009) and Uhlig et al., “Differential Activity of IL-12 and IL-23 in Mucosal and Systemic Innate Immune Pathology,” Immunity 25:309-318 (2006). The cytokine Granulocyte Macrophage-Colony Stimulating Factor (GM-CSF) reportedly plays a dual role in intestinal inflammation and was shown to have both protective and inflammatory properties in CD. Gathungu et al., “Granulocyte-macrophage Colony-Stimulating Factor Autoantibodies: A Marker of Aggressive Crohn's Disease,” Inflamatory Bowel Disease 19:1671-1680 (2013); Goldstein et al., “Defective Leukocyte GM-CSF Receptor (CD116) Expression and Function in Inflammatory Bowel Disease,” Gastroenterology 141:208-216 (2011); Griseri et al., “Granulocyte Macrophage Colony-Stimulating Factor-Activated Eosinophils Promote Interleukin-23 Driven Chronic Colitis,” Immunity 43:187-199 (2015); Han et al., “Loss of GM-CSF Signalling in Non-Haematopoietic Cells Increases NSAID Ileal Injury,” Gut 59:1066-1078 (2010); Han et al., “Granulocyte-macrophage Colony-Stimulating Factor Autoantibodies in Murine Ileitis and Progressive Ileal Crohn's Disease,” Gastroenterology 136:1261-1271 (2009); Hirata et al., “GM-CSF-facilitated Dendritic Cell Recruitment and Survival Govern the Intestinal Mucosal Response to a Mouse Enteric Bacterial Pathogen,” Cell Host & Microbe 7:151-163 (2010); Lang et al., “Transgenic Mice Expressing a Hemopoietic Growth Factor Gene (GM-CSF) Develop Accumulations of Macrophages, Blindness, and a Fatal Syndrome of Tissue Damage,” Cell 51:675-686 (1987); Nylund et al., “Granulocyte Macrophage-Colony-Stimulating Factor Autoantibodies and Increased Intestinal Permeability in Crohn Disease,” Journal of Pediatric Gastroenterology and Nutricion 52:542-548 (2011); Pearson et al., “ILC3 GM-CSF Production and Mobilisation Orchestrate Acute Intestinal Inflammation,” eLife 5:e10066 (2016); Sainathan et al., “Granulocyte Macrophage Colony-Stimulating Factor Ameliorates DSS-induced Experimental Colitis,” Inflammatory Bowel Diseases 14:88-99 (2008); and Song et al., “Unique and Redundant Functions of NKp46+ILC3s in Models of Intestinal Inflammation,” The Journal of Experimental Medicine 212:1869-1882 (2015). An important source of GM-CSF in the mucosa are group 3 innate lymphoid cells (ILC3), which act together with myeloid immune cells to sustain local intestinal immune homeostasis through microenvironmental signals. Kinnebrew et al., “Interleukin 23 Production by Intestinal CD103(+)CD11b(+) Dendritic Cells in Response to Bacterial Flagellin Enhances Mucosal Innate Immune Defense,” Immunity 36:276-287 (2012); Mortha et al., “Microbiota-dependent Crosstalk Between Macrophages and ILC3 Promotes Intestinal Homeostasis,” Science 343:1249288 (2014); and Klose and Artis, “Innate Lymphoid Cells as Regulators of Immunity, Inflammation and Tissue Homeostasis,” Nature Immunology 17:765-774 (2016). GM-CSF-produced by ILC3 promotes anti-bacterial and immunomodulatory myeloid cell functions at mucosal surfaces. Hamilton and Anderson, “GM-CSF Biology,” Growth Factors 22:225-231 (2004); Greter et al., “GM-CSF Controls Nonlymphoid Tissue Dendritic Cell Homeostasis but Is Dispensable for the Differentiation of Inflammatory Dendritic Cells,” Immunity 36:1031-1046 (2012); Stanley et al., “Granulocyte/macrophage Colony-Stimulating Factor-Deficient Mice Show No Major Perturbation of Hematopoiesis but Develop a Characteristic Pulmonary Pathology,” Proceedings of the National Academy of Sciences of the United States of America 91:5592-5596 (1994); and Bogunovic et al., “Origin of the Lamina Propria Dendritic Cell Network,” Immunity 31: 513-525 (2009). In a feedback loop, myeloid cells produce the metabolite retinoic acid (RA) which controls the transcriptional stability of ILC3 in healthy tissues. Aychek and Jung, “Immunology. The Axis of Tolerance,” Science 343:1439-1440 (2014). However, in CD patients, myeloid cells can promote ILC3 de-differentiation into inflammatory group 1 ILC (ILC1) via Interleukin(IL)-12 and IL-23. Vonarbourg et al., “Regulated Expression of Nuclear Receptor RORgammat Confers Distinct Functional Fates to NK Cell Receptor-Expressing RORgammat(+) Innate Lymphocytes,” Immunity 33:736-751 (2010); Bernink et al., “Interleukin-12 and -23 Control Plasticity of CD127(+) Group 1 and Group 3 Innate Lymphoid Cells in the Intestinal Lamina Propria,” Immunity 43:146-160 (2015); Bernink et al., “Human type 1 Innate Lymphoid Cells Accumulate in Inflamed Mucosal Tissues,” Nature Immunology 14:221-229 (2013); Buonocore et al., “Innate Lymphoid Cells Drive Interleukin-23-dependent Innate Intestinal Pathology,” Nature 464:1371-1375 (2010); Spencer et al., “Adaptation of Innate Lymphoid Cells to a Micronutrient Deficiency Promotes Type 2 Barrier Immunity,” Science 343:432-437 (2014); Kim et al., “Retinoic Acid Differentially Regulates the Migration of Innate Lymphoid Cell Subsets to the Gut,” Immunity 43:107-119 (2015); and Goverse et al., “Vitamin A Controls the Presence of RORgamma+Innate Lymphoid Cells and Lymphoid Tissue in the Small Intestine,” Journal of Immunology 196:5148-5155 (2016). The sources, kinetics, and dynamics of tissue-specific growth factors in the human mucosa are not well defined. It has been demonstrated (Mortha et al., “Microbiota-dependent Crosstalk Between Macrophages and ILC3 Promotes Intestinal Homeostasis,” Science 343:1249288 (2014); Magri et al., “Innate Lymphoid Cells Integrate Stromal and Immunological Signals to Enhance Antibody Production by Splenic Marginal Zone B Cells,” Nature Immunology 15:354-364 (2014); and Croxatto et al., “Group 3 Innate Lymphoid Cells Regulate Neutrophil Migration and Function in Human Decidua,” Mucosal Immunology 9:1372-1383 (2016)) that tissue-resident RORyt-expressing ILC3 imprint myeloid effector functions (via production of RA, transforming growth factor-beta (TGF-β) and IL-10) in a GM-CSF-dependent manner. GM-CSF-stimulated dendritic cells (DC) and macrophages were further shown to contribute to the generation and maintenance of immunosuppressive regulatory T cells (Treg). Mortha et al., “Microbiota-dependent Crosstalk Between Macrophages and ILC3 Promotes Intestinal Homeostasis,” Science 343:1249288 (2014). Deficiency in ILC3, similar to deficiency in GM-CSF, significantly impairs anti-microbial immunity and mucosal homeostasis. Hirata et al., “GM-CSF-facilitated Dendritic Cell Recruitment and Survival Govern the Intestinal Mucosal Response to a Mouse Enteric Bacterial Pathogen,” Cell Host & Microbe 7:151-163 (2010); Mortha et al., “Microbiota-dependent Crosstalk Between Macrophages and ILC3 Promotes Intestinal Homeostasis,” Science 343:1249288 (2014); Magri et al., “Innate Lymphoid Cells Integrate Stromal and Immunological Signals to Enhance Antibody Production by Splenic Marginal Zone B Cells,” Nature Immunology 15:354-364 (2014); and Kasahara et al., “Role of Granulocyte-Macrophage Colony-Stimulating Factor Signaling in Regulating Neutrophil Antifungal Activity and the Oxidative Burst During Respiratory Fungal Challenge,” Journal of Infectious Diseases 213:1289-1298 (2016). Recent reports identifying genetic loss-of-function mutations in the GM-CSF receptor of IBD patients further support an important protective role for GM-CSF signaling in CD. Chuang et al., “A Frameshift in CSF2RB Predominant Among Ashkenazi Jews Increases Risk for Crohn's Disease and Reduces Monocyte Signaling via GM-CSF,” Gastroenterology 151:710-723 (2016). Administration of yeast-produced recombinant GM-CSF (Sargramostim) was shown to improve IBD and ameliorate symptoms in several reports. Han et al., “Loss of GM-CSF Signalling in Non-Haematopoietic Cells Increases NSAID Ileal Injury,” Gut 59:1066-1078 (2010); Sainathan et al., “Granulocyte Macrophage Colony-Stimulating Factor Ameliorates DSS-induced Experimental Colitis,” Inflammatory Bowel Diseases 14:88-99 (2008); and Dabritz, “Granulocyte Macrophage Colony-Stimulating Factor and the Intestinal Innate Immune Cell Homeostasis in Crohn's Disease,” American Journal of Physiology. Gastrointestinal and Liver Physiology 306:G455-465 (2014). However, larger trials with recombinant GM-CSF for CD failed to reach statistical significance, maybe due to exceptionally high placebo group responses and suboptimal study design. Dabritz et al., “Reprogramming of Monocytes by GM-CSF Contributes to Regulatory Immune Functions During Intestinal Inflammation,” Journal of Immunology 194:2424-2438 (2015); Dieckgraefe and Korzenik, “Treatment of Active Crohn's Disease with Recombinant Human Granulocyte-Macrophage Colony-Stimulating Factor,” Lancet 360:1478-1480 (2002); Korzenik et al., “Sargramostim for Active Crohn's Disease,” The New England Journal of Medicine 352:2193-2201 (2005); and Roth et al., “Sargramostim (GM-CSF) for Induction of Remission in Crohn's Disease: A Cochrane Inflammatory Bowel Disease and Functional Bowel Disorders Systematic Review of Randomized Trials,” Inflammatory Bowel Diseases 18:1333-1339 (2012).

Besides genetic and epigenetic GM-CSF signaling defects, neutralizing anti—GM-CSF autoantibodies may also be contributing to disease. Such autoantibodies are thought to cause pulmonary alveolar proteinosis (PAP), resulting in a deficiency in alveolar macrophages and increased pulmonary pathologies. Piccoli et al., “Neutralization and Clearance of GM-CSF by Autoantibodies in Pulmonary Alveolar Proteinosis,” Nature Communications 6:7375 (2015) and Bonfield et al., “PU.1 Regulation of Human Alveolar Macrophage Differentiation Requires Granulocyte-Macrophage Colony-Stimulating Factor,” American Journal of Physiology. Lung Cellular and Molecular Physiology 285:L1132-1136 (2003). Importantly, anti—GM-CSF autoantibodies are found in a subset of CD patients and are associated with ileal involvement, higher disease severity, relapse and increased complications during the course of disease. Gathungu et al., “Granulocyte-macrophage Colony-Stimulating Factor Autoantibodies: A Marker of Aggressive Crohn's Disease,” Inflamatory Bowel Disease 19:1671-1680 (2013); Han et al., “Granulocyte-macrophage Colony-Stimulating Factor Autoantibodies in Murine Ileitis and Progressive Ileal Crohn's Disease,” Gastroenterology 136:1261-1271 (2009); Nylund et al., Granulocyte Macrophage-Colony-Stimulating Factor Autoantibodies and Increased Intestinal Permeability in Crohn Disease,” Journal of Pediatric Gastroenterology and Nutricion 52:542-548 (2011); Jurickova et al., “Pediatric Crohn Disease Patients With Stricturing Behaviour Exhibit Ileal Granulocyte-Macrophage Colony-Stimulating Factor (GM-CSF) Autoantibody Production and Reduced Neutrophil Bacterial Killing and GM-CSF Bioactivity,” Clinical and Experimental Immunology 172:455-465 (2013); and Dabritz et al., “Granulocyte Macrophage Colony-Stimulating Factor Auto-Antibodies and Disease Relapse in Inflammatory Bowel Disease,” American Journal of Gastroenterology 108:1901-1910 (2013). Intriguingly, it remains unclear why PAP patients do not display intestinal pathologies and why CD patients do not show PAP-associated pulmonary symptoms. Furthermore, both GM-CSF overproduction and the absence of GM-CSF, significantly increase the susceptibility to develop IBD, emphasizing the heterogeneity in CD pathogenesis. Griseri et al., “Granulocyte Macrophage Colony-Stimulating Factor-Activated Eosinophils Promote Interleukin-23 Driven Chronic Colitis,” Immunity 43:187-199 (2015); Han et al., “Loss of GM-CSF Signalling in Non-Haematopoietic Cells Increases NSAID Ileal Injury,” Gut 59:1066-1078 (2010); Hirata et al., “GM-CSF-facilitated Dendritic Cell Recruitment and Survival Govern the Intestinal Mucosal Response to a Mouse Enteric Bacterial Pathogen,” Cell Host & Microbe 7:151-163 (2010); Lang et al., “Transgenic Mice Expressing a Hemopoietic Growth Factor Gene (GM-CSF) Develop Accumulations of Macrophages, Blindness, and a Fatal Syndrome of Tissue Damage,” Cell 51:675-686 (1987); Pearson et al., “ILC3 GM-CSF Production and Mobilisation Orchestrate Acute Intestinal Inflammation,” eLife 5:e10066 (2016); Sainathan et al., “Granulocyte Macrophage Colony-Stimulating Factor Ameliorates DSS-induced Experimental Colitis,” Inflammatory Bowel Diseases 14:88-99 (2008); and Song et al., “Unique and Redundant Functions of NKp46+ILC3s in Models of Intestinal Inflammation,” The Journal of Experimental Medicine 212:1869-1882 (2015). These contradictory findings require further investigation to determine whether anti— GM-CSF autoantibodies in IBD patients are a consequence or a cause of their disease and further support the importance of GM-CSF as a potential drug for the treatment of Crohn's Disease. A more detailed diagnosis and classification of patients suitable for GM-CSF treatment are needed.

The present disclosure is directed to overcoming these and other deficiencies in the art.

### SUMMARY

A first aspect of the present disclosure relates to a composition comprising a post-translationally modified Granulocyte Macrophage-Colony Stimulating Factor (GM-C SF) protein.

A second aspect of the present disclosure relates to a method for diagnosing inflammatory bowel disease in a subject. The method includes contacting a sample from a subject with a reagent comprising the composition described herein. The method further includes detecting presence or absence of anti-Granulocyte Macrophage-Colony Stimulating Factor (GM-CSF) autoantibodies in the sample based on said contacting and diagnosing the inflammatory bowel disease in the subject based on said detecting.

A third aspect of the present disclosure relates to a method for diagnosing a pre-disease state of Crohn's Disease in a subject. The method includes contacting a sample from a subject with a reagent comprising the composition described herein. The method further includes detecting presence or absence of anti-Granulocyte Macrophage-Colony Stimulating Factor (GM-CSF) autoantibodies in the sample based on said contacting and diagnosing the pre-disease state of Crohn's Disease in the subject based on said detecting.

A fourth aspect of the present disclosure relates to a method of preventing or treating Crohn's Disease and/or a condition resulting from Crohn's Disease in a subject. The method includes selecting a subject having or at risk of having Crohn's Disease and administering a recombinant Granulocyte Macrophage-Colony Stimulating Factor (GM-CSF) protein to the selected subject under conditions effective to prevent or treat Crohn's Disease and/or a condition resulting from Crohn's Disease in the subject.

A fifth aspect of the present disclosure relates to a method for diagnosing and/or predicting severity of and/or treating Crohn's Disease in a subject. The method includes measuring a level of anti-Granulocyte Macrophage-Colony Stimulating Factor (GM-CSF) autoantibodies in a subject, wherein the measured level of anti-GM-C SF autoantibodies in the subject diagnoses Crohn's Disease and/or predicts the severity of the Crohn's Disease, and administering a recombinant Granulocyte Macrophage-Colony Stimulating Factor (GM-CSF) protein to the diagnosed subject.

A sixth aspect of the present disclosure relates to a method for diagnosing and/or predicting severity of and/or treating Crohn's Disease. The method includes detecting a glycoprofile of GM-CSF in a sample, and diagnosing Crohn's Disease and/or predicting the severity of Crohn's Disease based on said detecting.

Here, it is demonstrated that auto-reactive antibodies to GM-CSF in Crohn's Disease (CD) patients occur years before the onset of disease and alter myeloid cell homeostasis.

Anti-GM-CSF autoantibodies are detected in Crohn's Disease (CD) patients with severe disease manifestation (complicated disease at diagnosis). Using longitudinally collected sera from a military risk-factor cohort, naturally occurring anti-GM-CSF autoantibodies are characterized in a subset of CD patients years before disease onset. These anti-GM-CSF autoantibodies, including IgA, are neutralizing, target unique post-translational modifications, and their early presence is associated with complicated CD at presentation. By impairing an ILC3-GM-CSF-myeloid cell axis in the inflamed mucosa of CD patients, it is proposed that anti— GM-CSF autoantibodies negatively regulate gut myeloid homeostasis and thereby promote a “pre-diseased” tissue-resident immune state in CD.

Here, an analysis of 1800 serum samples is provided, collected longitudinally over a 10-year period as part of a prospective risk-factor cohort at the Department of Defense, from subjects who eventually developed CD (n=220), UC (n=200), or from matched individuals remaining healthy (HD, n=200). Using a combination of ELISA, Western Blot, and multiplexed Mass Cytometry assays, the titers, isotype profiles, epitopes, and function of anti-GM-CSF autoantibodies were analyzed. It is shown that anti-GM-CSF autoantibodies occur up to 2000 days prior to diagnosis in asymptomatic subjects developing CD, and are associated with higher risk of severe disease at presentation (HR=2.9 by log rank, p<0.001). By recognizing a set of post-translational modifications on GM-CSF, IgA- and IgG2-dominant anti-GM-CSF autoantibodies from CD patients impair communication of ILC3 and myeloid cell across the GM-CSF-GM-CSFR axis in the inflamed CD mucosa, and may thereby promote an imbalanced immune state fostering the establishment of a “pre-diseased” CD tissue state. The results described herein identify a subgroup of individuals at high risk of developing severe CD, and show novel mechanisms of pathology that may be harnessed for novel CD therapies.

The present disclosure includes results from two independent cohorts of IBD patients identified that approximately 30% of all CD patients present with detectable levels of anti-GM-CSF autoantibodies. These antibodies first bind to post-translational modifications on GM-CSF; second, are of mucosal isotypes (IgA, IgM, and IgG); and third, are detectable in the serum years before CD is diagnosed. These findings suggest that modifications of GM-CSF leading to the removal of the antibody epitopes will generate a cytokine that is suitable for the use in all CD patients including those with anti-GM-CSF autoantibodies and that specific diagnostic assays may be useful as a biomarker for this devastating pathology.

To tackle the gap in diagnostic and therapeutic tools, genetically engineered variants of the human cytokine GM-CSF were generated. Using site-directed mutagenesis, 6 known glycosylation sites were mutated either individually or simultaneously. The newly generated variants of GM-CSF, thus, either lack all or site-specific post-translationally added glycosylations when expressed. In parallel to this, it was identified that approximately 30% of all patients diagnosed with CD have detectable levels of anti-GM-CSF autoantibodies in their sera. Interestingly, these antibodies are detectable long before the diseases shows clinical manifestation and neutralized GM-CSF, resulting in an impaired GM-CSF receptor activity in mononuclear phagocytes (MNP). It was further identified that CD-associated, anti-GM-CSF autoantibodies recognize exclusively post-translational glycosylations on GM-CSF. Using enzymatically de-glycosylated forms of GM-CSF, lacking all post-translationally added sugar chains it was demonstrated that deglycosylated GM-CSF is able to engage the GM-CSF receptor on MNP and induce a normal signaling cascade, demonstrating bioactivity. More striking, deglycosylated GM-CSF is able to escape the neutralizing effects of anti-GM-CSF autoantibodies in CD patients. The findings of the present disclosure thus show that modified variants of GM-CSF are a potential therapeutic drug for the treatment of CD patients presenting with anti-GM-CSF autoantibodies.

Considering the advancement in modern medicine, diagnosis of CD is still in its infancies and only able to determine accurate diagnosis, if a full-blown clinical manifestation is presented. Serological diagnostic tools that allow a precise diagnosis of CD are virtually absent and likely challenging to obtain for all CD patients, given the overwhelming heterogeneity of this disease. With these findings that GM-CSF autoantibodies are detectable years before the onset of disease, it is believed that an ELISA-based assay, specific to these antibodies could serve as a tool to faithfully predict CD in a subgroup of patients and more specifically those who will present with a complication at diagnosis.

While ELISAs against anti-GM-CSF antibodies are easily to set up with commercially available reagents, ELISAs that allow the discrimination of glycosylation-specific anti-GM-CSF autoantibodies are not available. The genetically designed GM-CSF variants of the present disclosure are a perfect tool to establish such an ELISA-based assay to faithfully diagnose IBD patients using the sera of patients or even predict the risk of developing CD when performed routinely in geographic areas high CD prevalence.

The genetically modified variants of GM-CSF lacking specific post-translational glycosylations were designed to carry a cleavable protein-tag that allows efficient purification and enrichment for testing in vivo. Once removed, the purified recombinant variants of human GM-CSF lacking site specific of all post-translational modifications could be used for in vivo applications.

These variants of GM-CSF are a foundation of future tools for the personalized diagnosis and treatment of a larger subgroup of Crohn's Disease patients. With diagnostic tools in hands, diagnosis of a specific group of Crohn's Disease patients and recombinant proteins at site, tailored to escape the neutralizing effect of their autoantibodies, it is believed that this approach and tools have a great potential to be developed in two types of products.

An ELISA for the recognition of Crohn's Disease-specific anti-GM-CSF autoantibodies and the prediction of developing CD in healthy patients using this ELISA is described herein. Mass/Flow Cytometry based assay for the personalized characterization of CD-specific anti-GM-CSF patients is also described herein. Recombinant variants of human GM-CSF capable to escape anti-GM-CSF recognition for therapy are further described herein.

### BRIEF DESCRIPTION OF THE DRAWINGS

FIGS. 1A-1E depict the characterization of anti-GM-CSF autoantibodies in CD patients. In FIG. 1A, serum from healthy donors (HD), pulmonary alveolar proteinosis (PAP), CD, and ulcerative colitis (UC) patients were analyzed for their anti-GM-CSF antibody titers using anti-GM-CSF ELISA. Reciprocal titers for total IgG are shown and indicate predominant anti-GM-CSF antibodies in CD and in positive control PAP compared to UC and HD. FIG. 1B shows isotype profiles of individual patient sera from the PAP and CD patient group that were determined using anti-GM-CSF ELISA and isotype-specific HRP-conjugated secondary antibodies (anti-pan Ig, anti-IgG1, anti-IgG2, anti-IgG3, anti-IgG4, anti-IgA, anti-IgM, and anti-IgE). Heat maps show results of all patients tested in FIG. 1A. Each horizontal row represents one individual patient. Vertical rows indicate reciprocal titers for the indicated isotype. CD differed from PAP in having a predominant IgG2 and IgA profile. In FIG. 1C, recombinant GM-CSF and enzymatically stripped GM-CSF were separated using native PAGE. Western blots were generated and membranes probed with either polyclonal anti-human GM-CSF antibodies or sera from CD and PAP patients. Membranes were developed using pan anti-human-Ig-AP or anti-human-IgA-AP antibodies. CD patients reacted with post-translationally modified forms of GM-CSF, even after stripping large sugars. In FIG. 1D, plots show quantification of pSTAT5 signal in DC, MP, and pDC either left unstimulated or stimulated for 20 minutes with GM-CSF pre-incubated with serum from the indicated patient groups. Results indicate neutralizing activity of serum samples from CD with auto-GM-CSF antibodies, by reduction of pSTAT5 signaling. In FIG. 1E, loss in pSTAT5 signal intensity in the indicated cell population correlates with reciprocal titers of anti-GM-CSF antibodies in the indicated patient groups. One-way analysis of variance (ANOVA) Bonferroni's multiple comparison test was performed.

FIGS. 2A-2E show CD-specific anti-GM-CSF autoantibodies precede the onset of disease by years. FIG. 2A and FIG. 2B show reciprocal titers of anti-GM-CSF IgG and IgA autoantibodies in combined serum samples (training and validation cohort) at two time points prior to diagnosis and one time point post diagnosis, and frequency of samples with anti-GM-CSF antibodies was determined using a cutoff of titers >100 for positivity and shown as percentage at the bottom of dot plots. Anti-GM-CSF IgG and IgA autoantibodies were significantly higher as well as more frequent at all time points in CD samples compared to UC and HD samples, in both training and validations sets. In addition, within CD patients, titers and frequency increased as time of diagnosis neared. FIG. 2C shows a trajectory of anti-GM-CSF autoantibody titers. Blue lines indicate patients with anti-GM-CSF autoantibodies at the earliest time point of serum collection. Red lines indicate sero-converter, while black lines indicate patients negative for anti-GM-CSF autoantibodies. FIG. 2D shows risk hazard ratio to develop complications after diagnosis for patients with anti-GM-CSF autoantibodies (red) and without anti-GM-CSF autoantibodies 6 years prior to onset of disease (solid lines for IgG, dotted for IgA). In FIG. 2E, correlations of anti-GM-CSF autoantibodies with ASCA IgG and ASCA IgA antibodies at different time points prior to diagnosis are shown.

FIGS. 3A-3G illustrate that the inflamed CD mucosa shows impaired homeostatic functions in GM-CSF-responsive myeloid cells. Lamina propria leukocytes (LPL) were isolated from non-inflamed (NI) and inflamed (INF) ileal resection tissues. Cells were stained with an 28 marker-containing cocktail of antibodies (Table 2, as shown herein). FIG. 3A shows plots show relative distribution of leukocyte populations in NI (top plot) and INF (bottom plot) ileal CD resections material. Legend adjacent to plots show code for supervised color-coded labeling of populations. In FIG. 3B, whole LPL preparations from NI and INF ileal CD resection were either left untreated or stimulated with GM-CSF for 20 minutes, fixed, and stained with the same antibody cocktail used in FIG. 3A followed by intracellular staining for pSTAT5. Increase in signal intensity is visualized in t-SNE plots by changes in colors. Dark blue (low pSTAT5) and yellow/red (high pSTAT5). FIG. 3C depicts a heat map showing changes in STATS phosphorylation intensity after GM-CSF stimulation across myeloid populations in FIG. 3B. Myeloid cells (Dendritic cell subsets, DC and macrophages, MP) were identified using anti-CD45, anti-CD11 c, anti-HLA-DR, anti-CD14, anti-CD1c and anti-CD141 antibodies. FIG. 3D shows representative staining of NI and INF lamina propria cells from ileal CD resections. Macrophages were identified as CD45⁺CD11⁺HLA-DR⁺CD14⁺ and BDCA1 and BDCA3 dendritic cells were identified as CD45⁺CD11⁺HLA-DR⁺CD14⁻CD1⁺CD141⁻ or CD45⁺CD11⁺HLA-DR⁺CD14⁻CD1⁻CD141⁺ cells respectively. In. FIG. 3E, retinoic acid (RA) production was assessed in all APC using ALDEFLOUR staining on freshly isolated cells from NI and INF ileal CD resections. Mean fluorescence intensity (MFI) was quantified in CD45⁺CD11⁺HLA-DR⁺ cells of NI and INF tissues. In FIG. 3F, NI ileal CD biopsies from one control and one CSF2RB^{MUT} carrier were obtained and ALDEFLUOR staining was assessed in their myeloid CD45⁺CD11⁺HLA-DR⁺ populations. In FIG. 3G, INF ileal CD biopsies from one control and one CSF2RB^{MUT} carrier were obtained and ALDEFLUOR staining was assessed in their myeloid CD45⁺CD11⁺HLA-DR⁺ populations.

FIGS. 4A-4G depict innate and adaptive sources of intestinal GM-CSF in CD patients. Non-inflamed (NI) and inflamed (INF) ileal resection from CD patients were processed and leukocytes were isolated. Post isolation, cells were cultured ex vivo in complete media containing Brefeldin A for 4 hours. After culture, cell surfaces were stained with anti-CD45, anti-CD3, anti-CD4, anti-CD161, anti-CD127, anti-CD117 and anti-NKp44 antibodies prior to fixation and intracellular staining with anti-GM-CSF antibodies. Events in FIG. 4A show GM-CSF⁺CD45⁺ lamina propria cells. Dot plots adjacent show percentages of GM-CSF⁺CD45⁺ lamina propria cells in NI and INF resections. FIG. 4B shows GM-CSF⁺CD45⁺ amina propria cells that were analyzed for their expression of the surface markers CD3 and NKp44. Contour plots show representative staining of NI and INF resection tissue. Numbers within gates represent percentages. In FIG. 4C, plots show changes in percentages of NKp44⁺GM-CSF⁺CD45⁺ lamina propria cells and CD3⁺GM-CSF⁺CD45⁺ lamina propria cells within NI and INF resection tissue. In FIG. 4D, NKp44⁺ cells were analyzed for their expression of CD127, CD117, CD161, RORγt, and CD69. In FIG. 4E, NCR⁺ILC3 were identified as CD45⁺CD3⁻CD4⁻CD127⁺CD161⁺NKp44⁺CD117⁺ cells. GM-CSF production in NCR⁺ILC3 of NI and INF tissues was quantified using intracellular cytokine staining. Plots show percentages of GM-CSF⁺ cells within the NCRALC population of NI and INF tissues. In FIG. 4F, NCR⁺ILC3 were quantified within all ILCs (CD45⁺CD3⁻CD4⁻CD127⁺CD161⁺) as percentages of NKp44 +CD117⁺ cells. Plots adjacent to contour plots show quantification of NCRALC3. In FIG. 4G, INFy production was measured in ILC1/ex-NCR⁺ILC3 cells in NI and INF tissues. Plots adjacent to contour plots show quantification of IFNy +CD45 +CD3⁻CD4⁻CD127⁺CD16 NKp44⁻ILC1/ex-NCR⁺ILC3 cells. Datasets shown are representative of 4-20 individual ileal CD resections. Statistical analysis was performed using student's t-test. P values are indicated adjacent to datasets.

FIGS. 5A-5F show specificity and affinity of anti-GM-CSF autoantibodies in CD patients. As shown in FIG. 5A, ELISA specific to cytokines (G-CSF, IL-2 and GM-CSF), nuclear antigens and unrelated autoantigens was performed using sera from PAP patients and IBD patients. Sera simultaneously reacting against GM-CSF and other antigens were excluded from the study. In FIG. 5B, binding avidity of anti-GM-CSF autoantibodies was determined using anti-GM-CSF ELISA with titer-adjusted PAP and IBD sera. Post incubation of plates with anti-GM-CSF sera, wells were washed with buffers containing increasing amounts of NaCl. Signal detected by the ELISA plate reader post washing with high salt buffer was normalized to signals obtained in regular ELISAs. The binding strength of PAP sera (black) and IBD sera (red) are displayed. FIG. 5C shows anti-GM-CSF ELISA that were performed using CD, UC, and HC sera. Secondary antibodies recognizing IgG, IgA and IgM were used to identify enrichment of anti-GM-CSF autoantibodies in CD patients. In FIG. 5D, sera from CD patient were tested for their association with one of the three behavioral stages described in the Montreal Classification. In FIG. 5E, PBMCs were isolated from a buffy coat. For every tested serum, 2 million PBMCs were seeded into the well of a 96 well plate. Cells were either left unstimulated or stimulated with rhGM-CSF for 20 minutes. GM-CSF-stimulated samples were pre-incubated with either serum from CD patients, tested negative for anti-GM-CSF antibodies, anti-GM-CSF antibody-positive CD patients or sera from PAP patients. Cells were fixed after stimulation, barcoded using a combination of CD45-antibodies conjugated to different isotopes and intracellular barcodes. Prior to surface staining with a panel of surface marker (Table 2 as shown herein), cells were then pooled and stained for surface marker and intracellular pSTAT5. Phosphorylation of STAT5 analyzed in the indicated populations. Heat maps show signal intensity of anti-pSTAT5 staining in yellow color code for individual patients (lanes) within the indicated population (row). In FIG. 5F, PBMCs were stimulated in vitro with rhIL-3 in the presence or absence of the indicated sera for 20 minutes. Cells were washed, barcoded and stained with surface antibodies follow by Intracellular staining with antibodies against phosphorylated STAT5. Mass cytometry was performed and intensity of pSTAT5 was visualized using heat maps. Scatter plots show quantification of IL-3 mediated STAT5 phosphorylation in Basophils in the presence and absence of anti-GM-CSF autoantibodies in the CD sera.

FIGS. 6A-6C depict that anti-GM-CSF autoantibodies recognize native GM-CSF. FIG. 6A is a bar graph showing titers of anti-GM-CSF autoantibody ELISA on native and denaturated GM-CSF using sera from PAP and CD patients. FIG. 6B shows native PAGE of GM-CSF (Sargramostim) and stripped GM-CSF stained with Coomassie Brilliant Blue. FIG. 6C is a western blot of recombinantly expressed human GM-CSF purified from HEK293 cells stably secreting wild type human GM-CSF or human GM-CSF mutated to lack glycosylations.

FIGS. 7A-7F show that anti-GM-CSF autoantibodies precede the onset of CD. In FIGS. 7A-7D, anti-GM-CSF ELISA was performed using serum samples obtained from CD patients, UC patients, and HD, and FIGS. 7A-7B show the breakdown of data between a training and validation cohort. Sera were obtained at two (training cohort) and three (validation cohort) time points prior to diagnosis of disease and at time point after diagnosis of disease. Titers of anti-GM-CSF IgG and IgA were determined for each time point, and frequency was determined using a cutoff of titers >100 for positivity and shown as percentage at the bottom of dot plots. Anti-GM-CSF autoantibodies were significantly higher as well as more frequent at all time points in CD samples compared to UC and HD samples, in both training and validations sets. In addition, within CD patients, titers and frequency increased as time of diagnosis neared. Trajectory of anti-GM-CSF autoantibody titers in CD, UC and HD across different time points are displayed in FIG. 7E and FIG. 7F. Blue lines indicate patient tested positive at the earliest time point of collection. Red lines indicate sero-converter, while black lines indicate patients without anti-GM-CSF autoantibodies.

FIGS. 8A-8F show that anti-GM-CSF autoantibodies determine disease location and disease severity in two independent cohorts. In FIGS. 8A-8F, serum samples described in Table 3 as shown herein were analyzed for the association of IgG and IgA with disease location, Obstruction, Penetrance, Surgery, perianal involvement, and complications. A trainings cohort was established in FIGS. 8A-8C and compared to a validation cohort in FIGS. 8D-8F.

FIGS. 9A-9E show that the inflamed CD mucosa shows intact GM-CSFR expression but reduced homeostatic, GM-CSF-dependent myeloid functions. FIGS. 9A and 9B show CD116 and CD131 expression intensity across all leukocyte populations in NI (FIG. 9A) and INF (FIG. 9B) tissues identified by t-SNE analysis. Scale adjacent to plots indicates signal intensity. FIG. 9C is a histogram of representative ALDEFLOUR staining on intestinal macrophages from the NI (red) and INF (blue) CD mucosa. FIG. 9D depicts plots showing percentages of ALDEFLUOR staining⁺MP, CD141⁺DC and CD1⁺DC. In FIG. 9E, blood CD14⁺ monocytes were cultured in GM-CSF or M-CSF. Cells were analyzed for RA production using ALDEFLUOR staining 5 days later.

FIGS. 10A-10D show results of enzymatically treated GM-CSF, or genetically engineered GM-CSF lacking all posttranslational glycosylations. In FIG. 10A, purified CD14⁺ monocytes were stimulated with rhGM-CSF (Sargramostim) or stripped rhGM-CSF for 20 minutes. Cells were analyzed for pSTAT5 levels. In FIG. 10B, U937 myelomonocytic cells were analyzed for their expression of CD116 and CD131. Histograms show surface stained cells (blue) and unstained controls (grey). In FIG. 10C, U937 cells were stimulated with rhGM-CSF (Sargramostim), purified fully glycosylated GM-CSF or mutated GM-CSF lacking all posttranslational glycosylation sites. Following stimulation, pSTAT5 levels were analyzed. FIG. 10D plots show quantification of pSTAT5 signal intensity in monocytes and DC either stimulated with GM-CSF or stripped GM-CSF for 20 minutes pre-incubated with serum form the indicated patient groups. One-way analysis of variance (ANOVA) Bonferroni's multiple comparison test was performed.

FIG. 11 illustrates the establishment of a pre-diseased state through anti-GM-CSF autoantibodies in CD by model of CD development in anti-GM-CSF autoantibody carrying individuals. Scheme shows cellular crosstalk in healthy intestinal tissue. NCR⁺ILC3 produce GM-CSF that engages the GM-CSFR on myeloid cells to trigger the production of RA. Retinoic acid in turn stabilizes NCR⁺ILC3 and prevents excessive differentiation into IFN-y producing ex-RORγt NCR⁺ILC3/ILC1. In the presence of anti-GM-CSF autoantibodies, released by B cells, GM-CSF is neutralized and GM-CSFR signaling reduced, leading to a decreased production of retinoic acid. Consequently, decreased production of GM-CSF and increased differentiation into IFNy producing ex-RORγt NCR⁺ILC3/ILC1. The inflamed CD mucosa is characterized by decreased levels of GM-CSF produced by NCR⁺ILC3, reduced levels of RA from myeloid cells and excessive differentiation into IFNy producing ILC1/ex-ILC3.

FIG. 12 shows that heterodimeric GM-CSF (also referred to herein as “CSF2”) receptor is expressed on myeloid subsets and signals through JAK2/STATS which supports anti-fungal/viral and bacterial defense and supports immune tolerance.

FIG. 13 shows that human GM-CSF is glycosylated in its mature native form. Glycosylation sites on human GM-CSF (referred to interchangeably herein as “CSF2”), for example, include S22, S24, T27, S26, N44, and/or N54.

FIG. 14 shows that stable cell lines expressing human GM-CSF (i.e., CSF2) deficient in glycosylation sites are produced.

FIG. 15 shows stimulation of STATS phosphorylation in U937 cells with recombinant GM-CSF (i.e., CSF2).

FIG. 16 shows that recombinant human GM-CSF (i.e., CSF2) deficient in glycosylation sites is biologically active.

FIG. 17 shows that HIS-tag purification yields recombinant human GM-CSF (i.e., CSF2) from stable HEK293 clones lacking one or all glycosylations.

FIG. 18 shows that purification of recombinant human GM-CSF (i.e., CSF2) deficient in glycosylation does not alter biologically activity.

FIG. 19 shows a scheme demonstrating the workflow for pSTAT5 staining in samples.

FIGS. 20A-20C show that GM-CSF from CD patients display a differential profile of N-glycans. FIG. 20A is a schematic representation of N-glycan highlighting lectin recognition. FIG. 20B is a characterization of N-glycosylation of yeast- and CHO-producing recombinant GM-CSF by lectin blot for L-PHA, MALII, GNA and AAL as well as western blot for GM-CSF for the same recombinant GM-CSF. The WB for each lectin and GM-CSF were completed in different runs. M represents the protein molecular weight marker (kDa). FIG. 20C shows relative levels of L-PHA, GNA and AAL binding to GM-CSF from healthy donors (HD) and Crohn's Disease (CD) patients, normalized for the total levels of GM-CSF of each sample, as well as GM-CSF levels determined by ELISA using the same samples. Mann-Whitney test *p-value<0.05.

FIG. 21 shows the predictive performance of anti-flagellin X and ASCA-IgA antibody markers in terms of receiver operator curves (ROC) for years 1, 2, 3, 4, and 5 before diagnosis.

### DETAILED DESCRIPTION

A first aspect relates to a composition comprising a post-translationally modified Granulocyte Macrophage-Colony Stimulating Factor (GM-CSF) protein.

It is to be appreciated that certain aspects, modes, embodiments, variations, and features of the present disclosure are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this disclosure belongs.

As used herein, the term “about” means that the numerical value is approximate and small variations would not significantly affect the practice of the disclosed embodiments. Where a numerical limitation is used, unless indicated otherwise by the context, “about” means the numerical value can vary by ±1 or ±10% , or any point therein, and remain within the scope of the disclosed embodiments.

Where a range of values is described, it should be understood that intervening values, unless the context clearly dictates otherwise, between the upper and lower limit of that range and any other stated or intervening value in other stated ranges, may be used in the embodiments described herein.

As used herein, the terms “subject”, “individual”, or “patient,” are used interchangeably, and mean any animal, including mammals, such as mice, rats, other rodents, rabbits, dogs, cats, swine, cattle, sheep, horses, or primates, such as humans.

It is further appreciated that certain features described herein, which are, for clarity, described in the context of separate embodiments, can also be provided in combination in a single embodiment. Conversely, various features which are, for brevity, described in the context of a single embodiment, can also be provided separately or in any suitable sub-combination.

The post-translationally modified Granulocyte Macrophage-Colony Stimulating Factor (GM-CSF) protein may be derived from a variety of organisms, for example, a mammal, in particular a human, insect, yeast, and/or bacteria. A post translationally modified GM-CSF protein may be one or more recombinant proteins produced, including but not limited to Molgramostim (E.coli), Sargramostim (Yeast), and Regramostim (Hamster).

GM-CSF protein (referred to interchangeably herein as Colony-stimulating factor 2 (“CSF2”)) is important for the survival, differentiation, and function of mononuclear phagocytes (MNPs). GM-CSF in accordance with the present disclosure is produced by T cells, innate lymphoid cells (ILC), and stromal cells, for example. GM-CSF signals via signal transducer and activator of transcription, STATS. Heterodimeric CSF2 receptor in accordance with the present disclosure may be expressed on myeloid subsets and signals through JAK2/STAT5 which supports anti-fungal/viral defense, bacterial defense, and immune tolerance.

Human GM-CSF is glycosylated in its mature native form. Glycosylation is generally considered to be important for protein structure, function, and stability (half-life). Protein glycosylation, the enzymatic process that attaches oligosaccharides to amino acid sidechains, is among the most abundant and complex post-translational modifications in nature and plays critical roles in human health. See Kightlinger et al., “A Cell-free Biosynthesis Platform for Modular Construction of Protein Glycosylation Pathways,” Nature Communications 10:5404 (2019), which is hereby incorporated by reference in its entirety. Glycosylation sites on human GM-CSF (i.e., CSF2), for example, include S22, S24, T27, S26, N44, and/or N54.

Once nucleic acid sequence and/or amino acid sequence information is available for a native protein (e.g., a native GM-CSF protein), a variety of techniques become available for producing virtually any mutation in the native sequence. Botstein et al., “Strategies and Applications of In Vitro Mutagenesis,” Science 229:1193-1201 (1985), which is hereby incorporated by reference in its entirety, reviews techniques for mutating nucleic acids. Mutants of native GM-CSFs, for example, may be produced by site-specific oligonucleotide-directed mutagenesis (see, e.g., Zoller et al., “Oligonucleotide-directed Mutagenesis of DNA Fragments Cloned Into M13 Vectors,” Methods in Enzymology 100:468-500 (1983) and U.S. Patent 4,518,584, both of which are hereby incorporated by reference in their entirety); direct synthesis by solid phase methods, (see, e.g., Clark-Lewis et al., “Automated Chemical Synthesis of a Protein Growth Factor for Hemopoietic Cells, interleukin-3,” Science 231:134-139 (1986) and Doescher, M., “Solid-Phase Peptide Synthesis,” Meth. Enzymol. 47:578-617 (1977), which are hereby incorporated by reference in their entirety); or by so-called “cassette” mutagenesis described by Wells et al., “Cassette Mutagenesis: An Efficient Method for Generation of Multiple Mutations at Defined Sites,” Gene 34:315-323 (1985), Estell et al., “Probing Steric and Hydrophobic Effects on Enzyme-Substrate Interactions by Protein Engineering,” Science 233:659-63 (1986), and Mullenbach et al., “Chemical Synthesis and Expression in Yeast of a Gene Encoding Connective Tissue Activating peptide-III. A Novel Approach for the Facile Assembly of a Gene Encoding a Human Platelet-Derived Mitogen,” J. Biol. Chem. 261:719-22 (1986), all of which are hereby incorporated by reference in their entirety.

Mutants of a naturally occurring GM-CSF may be desirable in a variety of circumstances. For example, undesirable side effects might be shown less by certain mutants, particularly if the side effect is associated with a different part of the polypeptide from that of the desired activity. In some expression systems, a native polypeptide may be susceptible to degradation by proteases. In such systems, selected substitutions and/or deletions of amino acids which change the susceptible sequences can significantly enhance yields. Mutations to proteins may also increase yields in purification procedures and/or increase shelf lives of proteins by eliminating amino acids susceptible to oxidation, acylation, alkylation, or other chemical modifications. In bacterial expression systems, yields can sometimes be increased by eliminating or replacing conformationally inessential cysteine residues (see, e.g., U.S. Pat. No. 4,518,584, which is hereby incorporated by reference in its entirety).

The present disclosure, in one embodiment, may relate to polypeptides with conservative amino acid substitutions, insertions, and/or deletions with respect to the mature native GM-CSF sequence. “Conservative” as used herein includes that the alterations are as conformationally neutral as possible, i.e., they are designed to produce minimal changes in the tertiary structure of the mutant polypeptides as compared to the native GM-CSF, and that the changes are as antigenically neutral as possible, i.e., they are designed to produce minimal changes in the antigenic determinants of the mutant polypeptides as compared to the native GM-CSF. Conformational neutrality may be desirable for preserving biological activity, and antigenic neutrality may be desirable for avoiding the triggering of immunogenic responses in subjects treated with the compounds of the present disclosure. Guidelines exist which can allow those skilled in the art to make alterations that have high probabilities of being conformationally and antigenically neutral if desired. Some of the those guidelines include: substitution of hydrophobic residues is less likely to produce changes in antigenicity because they are likely to be located in the protein's interior; substitution of physiochemically similar residues has a lower likelihood of producing conformational changes because the substituting amino acid can play the same structural role as the replaced amino acid; alteration of evolutionarily conserved sequences is likely to produce deleterious conformational effects because evolutionary conservation suggests sequences may be functionally important, and negatively charged residues, for example, Asp and Glu, tend to be more immunogenic than neutral or positively charged residues (see Geysen et al., “Chemistry of Antibody Binding to a Protein,” Science 235:1184-90 (1987), which is hereby incorporated by reference in its entirety).

One example of a mutation that is associated to Crohn's Disease (CD), for example, is a rare mutation of the GM-CSF (i.e., CSF2) receptor. See, e.g., Chuang et al., “A Frameshift in CSF2RB Predominant Among Ashkenazi Jews Increases Risk for Crohn's Disease and Reduces Monocyte Signaling via GM-CSF,” Gastroenterology 151:710-723 e712 (2016), which is hereby incorporated by reference in its entirety.

A mutation may, in one embodiment, lead to a truncation of the cytoplasmic tail of the beta chain. To evaluate the consequence of this mutation, myeloid cells may be isolated from patients without this mutation as well as patients carrying this mutation, then stimulation may be applied with varying concentrations of GM-CSF, then immunoblotting conducted for STAT5 phosphorylation. An aldefluor assay may be performed that measures retinoic acid production which is an important molecule in immune tolerance as another downstream readout for myeloid function. In one embodiment, a GM-CSF protein mutation has a functional impact on myeloid subsets.

While a post-translational modification may include, for example, a modification that occurs after translation, it may also include any protein that is capable of being modified before translation, during translation, and/or after translation. In certain embodiments, the conditions present may prevent glycosylation, and/or may prevent further modifications. Post-translational modifications as discussed herein include, for example, glycoprotein modification including glycoengineered proteins and proteins produced by custom glycosylation. Custom glycosylation systems and examples of glycoengineering are known in the art. For example, one such system, GlycoPRIME, which uses a cell-free platform for glycosylation pathway assembly by rapid in vitro mixing and expression, may be useful for controlling glycosylation and may be used to produce unique glycosylation motifs in a protein. See Kightlinger et al., “A Cell-free Biosynthesis Platform for Modular Construction of Protein Glycosylation Pathways,” Nature Communications 10:5404 (2019), which is hereby incorporated by reference in its entirety. Likewise, N-glycosyltransferases from Actinobacillus pleuropneumonias (ApNGT) have been shown to modify native and rationally designed glycosylation sites within eukaryotic proteins in vitro and in E. coli. See, e.g., Kightlinger et al., “A Cell-free Biosynthesis Platform for Modular Construction of Protein Glycosylation Pathways,” Nature Communications 10:5404 (2019), which is hereby incorporated by reference in its entirety. Arginine glycosylation systems are known to provide a target for intervention strategies in Salmonella or E. coli, and glycosyltransferase inhibitors have been identified that prevent NleB1 glycosylation of TRADD (which is an example target for SseK/NleB glycosyltransferases). Nothaft et al., “New Discoveries in Bacterial N-glycosylation to Expand the Synthetic Biology Toolbox,” Current Opinion in Chemical Biology 53:16-24 (2019), which is hereby incorporated by reference in its entirety.

Moreover, some cell free systems decouple the production of glycoprotein synthesis components and target glycoprotein production to allow customizable single-pot glycosylation reactions and screening, characterization, and optimization of glycosylation sequences for the underlying glycosyltransferases. See Nothaft et al., “New Discoveries in Bacterial N-glycosylation to Expand the Synthetic Biology Toolbox,” Current Opinion in Chemical Biology 53:16-24 (2019), which is hereby incorporated by reference in its entirety. The concept of customizable glycosylation reactions to control glycosylation conditions, thereby allowing for prevention of glycosylation, or, alternatively, selective deglycosylation of a particular protein (i.e., GM-CSF) either during, before, or after translation are all contemplated in the methods of the present disclosure. In one embodiment, the post-translationally modified GM-CSF protein is prevented from further modification. In one embodiment, the further modification that is prevented is one or more glycosylations.

In one embodiment of the present disclosure, the post-translationally modified GM-CSF protein is not glycosylated (i.e., every glycosylation site on GM-CSF protein is deglycosylated). In one embodiment of the present disclosure, the post-translationally modified GM-CSF protein comprises one or more deglycosylation sites on the GM-CSF. For example, each of the six glycosylation sites S22, S24, T27, S26, N44, and/or N54 may be deglycosylated. In another embodiment of the present disclosure, the post-translationally modified GM-CSF protein comprises at least one of S22, S24, T27, S26, N44, and/or N54. In another embodiment of the present disclosure, the post-translationally modified GM-CSF protein comprises one deglycosylation site on the GM-CSF protein. In another embodiment of the present disclosure, the post-translationally modified GM-CSF protein comprises two deglycosylation sites on the GM-CSF protein. In another embodiment of the present disclosure, the post-translationally modified GM-CSF protein comprises three deglycosylation sites on the GM-CSF protein. In yet another embodiment of the present disclosure, the post-translationally modified GM-CSF protein comprises four deglycosylation sites on the GM-CSF protein. In yet another embodiment of the present disclosure, the post-translationally modified GM-CSF comprises five deglycosylation sites on the GM-CSF protein. In another embodiment of the present disclosure, the post-translationally modified GM-CSF protein comprises six deglycosylation sites on the GM-CSF protein. For example, in the present disclosure stable cell lines expressing human GM-CSF protein (i.e., CSF2) deficient in all glycosylation sites may be produced. STATS phosphorylation in accordance with the present disclosure may be stimulated in U937 cells, for example, with recombinant human GM-CSF protein having one or more of its glycosylation sites deglycosylated.

The post-translationally modified GM-CSF protein may, in one embodiment, comprise a single modification at S22 glycosylation site, or a single modification at S24 glycosylation site, or a single modification at T27 glycosylation site, or a single modification at S26 glycosylation site, or a single modification at N44 glycosylation site, or a single modification at N54 glycosylation site. In another embodiment, the post-translationally modified GM-CSF protein may comprise two modifications at two of the following glycosylation sites: S22, S24, T27, S26, N44, and/or N54. In another embodiment, the post-translationally modified GM-CSF protein may comprise three modifications at three of the following glycosylation sites: S22, S24, T27, S26, N44, and/or N54. In another embodiment, the post-translationally modified GM-CSF protein may comprise four modifications at four of the following glycosylation sites: S22, S24, T27, S26, N44, and/or N54. In another embodiment, the post-translationally modified GM-CSF protein may comprise five modifications at five of the following glycosylation sites: S22, S24, T27, S26, N44, and/or N54. In another embodiment, the post-translationally modified GM-CSF protein may comprise six modifications at each of the following six glycosylation sites: S22, S24, T27, S26, N44, and N54. In one embodiment, the post-translational modification may comprise deglycosylation at the respective glycosylation site.

In accordance with the present disclosure, recombinant human GM-CSF (i.e., CSF2) deficient in glycosylation sites may be biologically active. HIS-tag purification as disclosed herein may yield recombinant human GM-CSF (i.e., CSF2) from stable HEK293 clones lacking one or all glycosylations. In one embodiment, purification of recombinant human GM-CSF (i.e., CSF2) deficient in glycosylation does not alter biological activity.

The post-translationally modified GM-CSF protein as described herein may be, for example, between about 1 kDa and about 100 kDa. For example, the GM-CSF protein may be about 1 kDa, about 5 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, and any amount therebetween. In other embodiments, the GM-CSF protein may be less than about 1 kDa or more than about 50 kDa. In one embodiment, the GM-CSF protein may be between about 18 kDa and about 30 kDa. In another embodiment, the GM-CSF protein may be about 15 kDa.

In one embodiment, the composition further comprises a pharmaceutically acceptable carrier. “Pharmaceutically acceptable carriers” as used herein refer to conventional pharmaceutically acceptable carriers See Remington's Pharmaceutical Sciences, by E. W. Martin, Mack Publishing Co., Easton, Pa., 15th Edition (1975), which is hereby incorporated by reference in its entirety, describes compositions suitable for pharmaceutical delivery of the inventive compositions described herein. In particular, a pharmaceutically acceptable carrier as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits. In one embodiment, the pharmaceutically acceptable carrier is selected from the group consisting of a liquid filler, a solid filler, a diluent, an excipient, a solvent, and an encapsulating material.

Pharmaceutically acceptable carriers (e.g., additives such as diluents, immunostimulants, adjuvants, antioxidants, preservatives and solubilizing agents) are nontoxic to the cell or subject being exposed thereto at the dosages and concentrations employed. Examples of pharmaceutically acceptable carriers include water, e.g., buffered with phosphate, citrate and another organic acid. Representative examples of pharmaceutically acceptable excipients that may be useful in the present disclosure include antioxidants such as ascorbic acid; low molecular weight (less than about 10 residues) polypeptides; proteins, such as serum albumin, gelatin, or immunoglobulins; adjuvants (selected so as to avoid adjuvant-induced toxicity, such as a (3-glucan as described in U.S. Pat. No. 6,355,625, which is hereby incorporated by reference in its entirety, or a granulocyte colony stimulating factor (GCSF)); hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt forming counterions such as sodium; and/or nonionic surfactants such as TWEEN®, polyethylene glycol (PEG), and PLURONICS®.

In addition, in various embodiments, the compositions according to the disclosure may be formulated for delivery via any route of administration. The route of administration may refer to any administration pathway known in the art, including but not limited to aerosol, nasal, oral, transmucosal, transdermal, subcutaneous, or parenteral. Parenteral refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or in the form of lyophilized powders.

The compositions according to the disclosure may be formulated as appropriate for such administration, which may be tailored to a given purpose, such as in a tablet, capsule, or other form for oral administration or injectable formulation for injection, or gel, cream, powder, ointment, or other composition for rectal or dermal application. Any suitable approach for delivery of composition can be utilized to practice this aspect. Typically, the composition will be administered to a patient in a vehicle that delivers the agent(s) to the target cell, tissue, or organ. Exemplary routes of administration include, without limitation, by intratracheal inoculation, aspiration, airway instillation, aerosolization, nebulization, intranasal instillation, oral or nasogastric instillation, intraperitoneal injection, intravascular injection, topically, transdermally, parenterally, subcutaneously, intravenous injection, intra-arterial injection (such as via the pulmonary artery), intramuscular injection, intrapleural instillation, intraventricularly, intralesionally, intracranially, intrathecally, intracerebroventricularly, intraspinally, by application to mucous membranes (such as that of the nose, throat, bronchial tubes, genitals, and/or anus), or implantation of a sustained release vehicle.

Some non-limiting examples include oral, parenteral, subcutaneous, intraperitoneal, intrapulmonary, intranasal, intracranial, and can be performed using an implantable device, such as an osmotic pump. Parenteral infusions include intramuscular, intravenous, intraarterial, intraperitoneal, pulmonary instillation as mist or nebulization, and subcutaneous administration. In one embodiment, the administering is carried out intraperitoneally, orally, parenterally, nasally, subcutaneously, intravenously, intramuscularly, intracerebroventricularly, intraparenchymally, by inhalation, intranasal instillation, by implantation, by intracavitary or intravesical instillation, intraocularly, intraarterially, intralesionally, transdermally, topically, intradermally, intrapleurally, intrathecally, or by application to mucous membranes.

In one embodiment, the composition may further comprise an adjuvant. Suitable adjuvants are known in the art and include, without limitation, flagellin, Freund's complete or incomplete adjuvant, aluminum hydroxide, lysolecithin, pluronic polyols, polyanions, peptides, oil emulsion, dinitrophenol, iscomatrix, and liposome polycation DNA particles. In one embodiment, the composition is formulated for the diagnosis and treatment of CD.

Another aspect of the present disclosure relates to a method for diagnosing inflammatory bowel disease in a subject. The method includes contacting a sample from a subject with a reagent comprising the composition described herein. The method further includes detecting presence or absence of anti-Granulocyte Macrophage-Colony Stimulating Factor (GM-CSF) autoantibodies in the sample based on said contacting and diagnosing the inflammatory bowel disease in the subject based on said detecting.

The inflammatory bowel disease may, in one example, be CD or UC or a combination of CD and UC. In one embodiment, the inflammatory bowel disease is selected from the group consisting of CD and UC. Some subjects in the context of this and other aspects described herein may have cancer and may have been administered an immune checkpoint blockade, which, in certain subjects may lead to the development of inflammatory bowel disease such as CD and/or UC. The methods and compositions described herein may be useful in treating such a subject (e.g., one who was treated with immune checkpoint blockade and having melanoma, which may in some instances lead to colitis as an adverse event).

GM-CSF autoantibodies as described herein may include, but are not limited to, anti-GM-CSF IgA, anti-GM-CSF IgG, anti-GM-CSF IgG1, anti-GM-CSF IgG2, anti-GM-CSF IgG3, anti-GM-CSF IgG4, and anti-GM-CSF IgM. In one embodiment, the GM-CSF autoantibodies are selected from the group consisting of anti-GM-CSF IgA, anti-GM-CSF IgG, anti-GM-CSF IgG1, anti-GM-CSF IgG2, anti-GM-CSF IgG3, anti-GM-CSF IgG4, and anti— GM-CSF IgM.

In one embodiment, the method further includes detecting the presence or absence of one or more additional marker. Examples of additional markers include but are not limited to anti-pANCA, ASCA, anti-CBir1 (flagellin), anti-OmpC (E. coli membrane), anti-A4 Fla2, and anti-FlaX.

Another aspect of the present disclosure relates to a method for diagnosing a pre-disease state of Crohn's Disease in a subject. The method includes contacting a sample from a subject with a reagent comprising the composition described herein. The method further includes detecting presence or absence of anti-Granulocyte Macrophage-Colony Stimulating Factor (GM-CSF) autoantibodies in the sample based on said contacting and diagnosing the pre-disease state of Crohn's Disease in the subject based on said detecting.

Homeostasis as described herein originates from the terms homoeos which refers to something “similar” and stasis which refers to something “standing stil.” Homeostasis as described herein includes the tendency toward a relatively stable equilibrium between interdependent elements, especially as maintained by physiological processes. In homeostatic conditions, there is for example, a balance between tolerance and inflammation. A loss of intestinal homeostasis as described herein includes, for example, an environment where there is a loss of balance leading to inflammation which exceeds tolerance. This loss of intestinal homeostasis may be found, for example, in severe chronic conditions such as inflammatory bowel disease (IBD).

Crohn's Disease (CD) is an example of IBD. Another example of IBD includes Ulcerative Colitis (UC). Crohn's disease in accordance with the present disclosure can include illeal CD, colonic CD, illeo-colic CD, and upper gastrointestinal CD. Ulcerative colitis may include, for example, ulcerative proctitis, left-sided colitis, and pancolitis.

CD is difficult to diagnose and distinguish from UC. CD as described herein includes, for example, a chronic inflammation in the gastrointestinal tract of a subject. Historically, it is difficult to treat CD and there is no known cure. Examples of standard treatments are limited to antibiotics, anti-inflammatory drugs, broad immunosuppression, and surgery. IBD may be caused by a number of factors, for example, genetic susceptibility, immune response, environmental triggers, and luminal microbial antigens and adjuvants. Generally, mononuclear phagocytes (MNP) maintain intestinal homeostasis and regulate the response to luminal and environmental antigens to promote functional heterogeneity.

Another aspect of the present disclosure relates to a method for diagnosing and/or predicting severity of and/or treating Crohn's Disease in a subject. The method includes measuring a level of anti-Granulocyte Macrophage-Colony Stimulating Factor (GM-CSF) autoantibodies in a subject, wherein the measured level of anti-GM-CSF autoantibodies in the subject diagnoses Crohn's Disease and/or predicts the severity of the Crohn's Disease, and administering a recombinant Granulocyte Macrophage-Colony Stimulating Factor (GM-CSF) protein to the diagnosed subject.

This aspect of the disclosure is carried out in accordance with the previously described aspects.

This aspect may be used to diagnose and/or predict severity of and/or treat CD in the presence of complicated disease with one or more stricture and/or one or more fistula/abscess. This aspect is useful in predicting complicated CD at time of diagnosis (i.e., in a subject presenting for the first time with a complication like a stricture and/or fistula/abscess). In one embodiment, the modified GM-CSF protein comprises a cleavable protein-tag. In another embodiment, the modified GM-CSF protein is purified.

For purposes of the present disclosure, specifically the previously described aspects that comprise detecting presence of absence of anti-Granulocyte Macrophage-Colony Stimulating Factor (GM-CSF) autoantibodies, the term “antibody” may include monoclonal antibodies, polyclonal antibodies, antibody fragments, genetically engineered forms of the antibodies, and combinations thereof. The term “antibody,” which is used interchangeably with the term “immunoglobulin,” includes full length (i.e., naturally occurring or formed by normal immunoglobulin gene fragment recombinatorial processes) immunoglobulin molecules (e.g., an IgG antibody) and immunologically active fragments thereof (i.e., including the specific binding portion of the full-length immunoglobulin molecule), which again may be naturally occurring or synthetic in nature. Accordingly, the term “antibody fragment” includes a portion of an antibody such as F(ab′)₂, F(ab)₂, Fab′, Fab, Fv, scFv, and the like. Regardless of structure, an antibody fragment binds with the same antigen that is recognized by the full-length antibody. Methods of making and screening antibody fragments are well-known in the art.

Naturally occurring antibodies typically have two identical heavy chains and two identical light chains, with each light chain covalently linked to a heavy chain by an inter-chain disulfide bond and multiple disulfide bonds further link the two heavy chains to one another. Individual chains may fold into domains having similar sizes (110-125 amino acids) and structures, but different functions. The light chain can comprise one variable domain (VL) and/or one constant domain (CL). The heavy chain can also comprise one variable domain (VH) and/or, depending on the class or isotype of antibody, three or four constant domains (CHI, CH 2, CH3 and CH4). In humans, the isotypes are IgA, IgD, IgE, IgG, and IgM, with IgA and IgG further subdivided into subclasses or subtypes (IgA1-2 and IgG1-4).

Generally, the variable domains show considerable amino acid sequence variability from one antibody to the next, particularly at the location of the antigen-binding site. Three regions, called hyper-variable or complementarity-determining regions (CDRs), are found in each of VL and VH, which are supported by less variable regions called framework variable regions. Antibodies include IgG monoclonal antibodies as well as antibody fragments or engineered forms. These are, for example, Fv fragments, or proteins wherein the CDRs and/or variable domains of the exemplified antibodies are engineered as single-chain antigen-binding proteins.

The portion of an antibody consisting of the VL and VH domains is designated as an Fv (Fragment variable) and constitutes the antigen-binding site. A single chain Fv (scFv or SCA) is an antibody fragment containing a VL domain and a VH domain on one polypeptide chain, wherein the N terminus of one domain and the C terminus of the other domain are joined by a flexible linker. The peptide linkers used to produce the single chain antibodies are typically flexible peptides, selected to assure that the proper three-dimensional folding of the VL and VH domains occurs. The linker is generally 10 to 50 amino acid residues, and in some cases is shorter, e.g., about 10 to 30 amino acid residues, or 12 to 30 amino acid residues, or even 15 to 25 amino acid residues. An example of such linker peptides includes repeats of four glycine residues followed by a serine residue.

Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies.

Fab (Fragment, antigen binding) refers to the fragments of the antibody consisting of the VL, CL, VH, and CH1 domains. Those generated following papain digestion simply are referred to as Fab and do not retain the heavy chain hinge region. Following pepsin digestion, various Fabs retaining the heavy chain hinge are generated. Those fragments with the interchain disulfide bonds intact are referred to as F(ab′)₂, while a single Fab′ results when the disulfide bonds are not retained. F(ab′)₂ fragments have higher avidity for antigen that the monovalent Fab fragments.

Fc (Fragment crystallization) is the designation for the portion or fragment of an antibody that comprises paired heavy chain constant domains. In an IgG antibody, for example, the Fc comprises CH2 and CH3 domains. The Fc of an IgA or an IgM antibody further comprises a CH4 domain. The Fc is associated with Fc receptor binding, activation of complement mediated cytotoxicity and antibody-dependent cellular-cytotoxicity (ADCC). For antibodies such as IgA and IgM, which are complexes of multiple IgG-like proteins, complex formation requires Fc constant domains.

Finally, the hinge region separates the Fab and Fc portions of the antibody, providing for mobility of Fabs relative to each other and relative to Fc, as well as including multiple disulfide bonds for covalent linkage of the two heavy chains.

Antibody “specificity” refers to selective recognition of an antibody for a particular epitope of an antigen. The term “epitope” includes any protein determinant capable of specific binding to an immunoglobulin or T-cell receptor or otherwise interacting with a molecule. Epitopic determinants generally consist of chemically active surface groupings of molecules such as amino acids or carbohydrate or sugar side chains and generally have specific three dimensional structural characteristics, as well as specific charge characteristics. An epitope may be “linear” or “conformational.” In a linear epitope, all of the points of interaction between the protein and the interacting molecule (such as an antibody) occur linearly along the primary amino acid sequence of the protein. In a conformational epitope, the points of interaction occur across amino acid residues on the protein that are separated from one another, i.e., noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen.

Monoclonal antibodies may be murine, human, humanized, or chimeric. A humanized antibody is a recombinant protein in which the CDRs of an antibody from one species; e.g., a rodent, rabbit, dog, goat, horse, or chicken antibody (or any other suitable animal antibody), are transferred into human heavy and light variable domains. The constant domains of an antibody molecule are derived from those of a human antibody. Methods for making humanized antibodies are well known in the art. Chimeric antibodies preferably have constant regions derived substantially or exclusively from human antibody constant regions and variable regions derived substantially or exclusively from the sequence of the variable region from a mammal other than a human. The chimerization process can be made more effective by also replacing the variable regions—other than the hyper-variable regions or the complementarity—determining regions (CDRs), of a murine (or other non-human mammalian) antibody with the corresponding human sequences. The variable regions other than the CDRs are also known as the variable framework regions (FRs).

As described herein, an “autoantibody” or an “autoimmune antibody” is an antibody produced by the immune system that is directed against one or more of the host's own proteins. Autoantibodies may be produced by a host's immune system when it fails to distinguish between self and non-self proteins. Typically, the immune system is able to discriminate by recognizing foreign substances (non-self) and ignoring the host's own cells (self). When an immune system in a subject stops recognizing one or more of the host's normal constituents as self, it may then produce autoantibodies that attack its own cells, tissues, and/or organs.

Methods for detecting the presence, or testing for the presence, of an autoantibody in a subject may be achieved a number of ways. Exemplary methods include, but are not limited to, protein microarrays, antibody-based (immunoassay-based) testing techniques (including Western blotting, immunoblotting, enzyme-linked immunosorbant assay (ELISA), “sandwich” immunoassays, radioimmunoassay (RIA), immunoprecipitation and dissociation-enhanced lanthanide fluoro-immuno assay (DELFIA), precipitin reactions, gel diffusion precipitin reactions, immunodiffusion assays, immunoradiometric assays and protein A immunoassays) proteomics techniques, surface plasmon resonance (SPR), versatile fibre-based SPR, chemiluminescence, fluorescent polarization, phosphorescence, immunohistochemistry, immunofluorescence, microcytometry, microscopy, fluorescence activated cell sorting (FACS), flow cytometry, mass spectrometry-based techniques (including liquid chromatography coupled to tandem mass spectrometry (LC-MS/MS), nano LC-MS/MS, matrix-assisted laser desorption/ionization mass spectrometry (MALDI-MS), as described for example in WO 2009/004576 which is hereby incorporated by reference in its entirety (including surface enhanced laser desorption/ionization mass spectrometry (SELDI-MS), especially surface-enhanced affinity capture (SEAC), surface-enhanced need desorption (SEND) or surface-enhanced photo label attachment and release (SEPAR)), and matrix-assisted laser desorption/ionization time-of-flight (MALDI-TOF) mass spectrometry. Examples of methods of detecting and measuring the level of anti-GM-CSF autoantibody in a sample are provided, for example, in U.S. Pat. Publ. No. 2010/0255513, which is incorporated herein by reference in its entirety.

Many techniques for detecting autoantibodies rely on an agent being detectably labelled. An agent is typically labelled by covalently or non-covalently combining the agent with a substance or ligand that provides or enables the generation of a detectable signal. Some examples include, but are not limited to, radioactive isotopes, enzymes, fluorescent substances, luminescent substances, ligands, microparticles, redox molecules, substrates, cofactors, inhibitors, and magnetic particles. Examples of radioactive isotopes include, but are not limited to ¹²⁵I, ¹³¹I, ³H , ¹²C, ¹³C, ³²P, ³⁶Cl, ⁵¹Cr, ⁵⁷CO, ⁵⁸CO, ⁵⁹Fe, ⁹⁰Y, and ¹⁸⁶Re. Examples of enzymes available as detection labels include, but are not limited to, peroxidase or alkaline phosphatase, β-glucuronidase, β-glucosidase, β-galactosidase, phosphofructokinase, urease, acetylcholinesterase, glucose oxidase, hexokinase and GDPase, RNase, glucose oxidase and luciferase, phosphoenolpyruvate carboxylase, aspartate aminotransferase, phosphenolpyruvate decarboxylase, and β-lactamase. Examples of fluorescent substances include, but are not limited to, rhodamine, phycoerythrin, fluorescin, isothiocyanate, phycocyanin, allophycocyanin, o-phthaldehyde, and fluorescamin. Examples of luminescent substances include acridinium esters, luciferin and luciferase. Examples of ligands include biotin and its derivatives. Examples of microparticles include colloidal gold and colored latex. Examples of the redox molecules include 1,4-benzoquinone, hydroquinone, ferrocene, ruthenium complexes, viologen, quinone, Ti ions, Cs ions, diimide, K₄W(CN)₈, [Os(bpy)₃]²⁺, [RU(bpy)₃]²⁺, and [MO(CN)₈]⁴⁻.

When the agent is an antigen from which the one or more autoantibodies are derived, antigen-autoantibody interactions can be detected using a number of the methods as described herein. In general, these methods rely on contacting a sample derived from a subject with a sample containing the corresponding antigen, or part thereof, under conditions which allow an immunospecific antigen-antibody binding reaction to occur. The antigen may be present in solution, or may be anchored to a solid support such that chip-based or microarray detection methods may be used. Generally, in a chip-based or microarray approach, a peptide having an amino acid sequence representing all, or a portion, of the antigen occupies a known location on a substrate. A sample that has been obtained from a subject may hybridized to the chip or microarray and binding of the corresponding autoantibody (if present) to the antigen is detected by, for example, mass spectrometry or an immunoassay-based assay. Protein microarrays are known in the art, for example, as described in U.S. Pat. Nos. 6,537,749 and 6,329,209, and WO 00/56934 and WO 03/048768, all of which are hereby incorporated by reference in their entirety.

As described herein, the presence of an autoantibody of interest can also be measured by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer, and hybrids of these. The mass spectrometer may be a laser desorption/ionization (LDI) mass spectrometer. In laser desorption/ionization mass spectrometry, the autoantibody or autoantibodies to be detected may be placed on the surface of a mass spectrometry probe, a device adapted to engage a probe interface of the mass spectrometer and to present the autoantibody or autoantibodies to ionizing energy for ionization and introduction into a mass spectrometer. A laser desorption mass spectrometer uses laser energy, for example from a laser that is ultraviolet, and also from an infrared laser, to desorb analytes from a surface, to volatilize and ionize them and make them available to the ion optics of the mass spectrometer. The analysis of autoantibodies by LDI can take the form of MALDI or of SELDI.

A SELDI method is described, for example, in U.S. Pat. Nos. 5,719,060 and 6,225,047, both of which are hereby incorporated by reference in their entirety. SELDI method relates to desorption/ionization gas phase ion spectrometry (e.g., mass spectrometry) where an analyte (in this instance one or more of the autoantibodies to be detected) is captured on the surface of a SELDI mass spectrometry probe. SELDI also encompasses affinity capture mass spectrometry, surface-enhanced affinity capture (SEAC) and immuno-capture mass spectrometry (icMS) as described by Penno et al., “Detection and Measurement of Carbohydrate Deficient Transferrin in Serum Using Immuno-Capture Mass Spectrometry: Diagnostic Applications for Annual Ryegrass Toxicity and Corynetoxin Exposure,” Res. Vet. Sci. 93:611-617 (2012), which is hereby incorporated by reference in its entirety. These platforms involve the use of probes that have a material on the probe surface that can capture an autoantibody through a non-covalent affinity interaction (adsorption) between the material and the autoantibody. The material may be referred to as an “adsorbent”, a “capture reagent”, an “affinity reagent” or a “binding moiety.” Probes may be called “affinity capture probes” and may have an adsorbent surface. The capture reagent may be any material that can bind an autoantibody. The capture reagent may be attached to the probe surface by physisorption or chemisorption. The probes, which may take the form of a functionalized biochip or magnetic bead, may have a capture reagent already attached to the surface, or the probes are pre-activated and include a reactive moiety that is capable of binding the capture reagent, for example, by a reaction forming a covalent or coordinate covalent bond.

A chromatographic adsorbent may be any adsorbent material used in chromatography. Chromatographic adsorbents include, for example, ion exchange materials, simple biomolecules (like nucleotides, amino acids, simple sugars, and fatty acids), metal chelators (such as nitrilotriacetic acid or iminodiacetic acid), immobilized metal chelates, hydrophobic interaction adsorbents, hydrophilic interaction adsorbents, dyes, and mixed mode adsorbents (like hydrophobic attraction or electrostatic repulsion adsorbents). One method which uses a chromatographic adsorbent is liquid chromatography coupled to tandem mass spectrometry (LC-MS/MS), including nano-LC-MS/MS.

A bio-specific adsorbent may include an adsorbent comprising a biomolecule, for example, a nucleic acid molecule, a polypeptide, a polysaccharide, a lipid, a steroid or a conjugate of these. In some examples, a bio-specific adsorbent may be a macromolecular structure such as a multiprotein complex, a biological membrane, or a virus. Examples of bio-specific adsorbents may include antibodies, receptor proteins, and nucleic acids. Biospecific adsorbents may have higher specificity for a target autoantibody than chromatographic adsorbents.

A probe with an adsorbent surface is typically contacted with a sample being tested for a period of time sufficient to allow the autoantibody or autoantibodies under investigation to bind to the adsorbent. After a period of incubation, a substrate may be washed to remove unbound material. Any suitable washing solutions may be used, including aqueous solutions. The amount of molecules that remain bound can be manipulated by adjusting the stringency of the wash. The elution characteristics of a wash solution may depend, for example, on pH, ionic strength, hydrophobicity, degree of chaotropism, detergent strength, and temperature. An energy absorbing molecule may be applied to the substrate.

In a further approach, autoantibodies may be captured with a solid-phase bound immuno-adsorbent that has antibodies that specifically bind to the or each autoantibody. After washing the adsorbent to remove unbound material, autoantibodies may be eluted from the solid phase and detected by applying them to a biochip that binds the autoantibodies.

An autoantibody which is bound to the substrate is detected in a gas phase ion spectrometer such as a time-of-flight mass spectrometer. An autoantibody may be ionized by an ionization source, like a laser. The generated ions may be collected by an ion optic assembly, and then a mass analyzer may disperse and analyze passing ions. A detector can then translate information of the detected ions into mass-to-charge ratios. Detection of an autoantibody may involve detection of signal intensity. Thus, both the quantity and mass of the autoantibody may be determined.

Another method of laser desorption mass spectrometry is known as surface-enhanced neat desorption (SEND). SEND uses probes having energy absorbing molecules that are chemically bound to the probe surface (SEND probe). Energy absorbing molecules (EAM) may include molecules that may absorb energy from a laser desorption/ionization source and then contribute to desorption and ionization of analyte molecules in contact therewith. EAM may include molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. The energy absorbing molecule may be incorporated into a linear or cross-linked polymer, for example, a polymethacrylate. SEND is described in U.S. Pat. No. 6,124,137 and WO 03/64594, both of which are hereby incorporated by reference in their entirety.

Another example of LDI is known as surface-enhanced photolabile attachment and Release (SEPAR). SEPAR involves using probes having moieties attached to the surface that can covalently bind an autoantibody, and then release the autoantibody through breaking a photolabile bond in the moiety after exposure to light, e.g. to laser light. SEPAR and other forms of SELDI are adaptable to detecting an autoantibody.

MALDI is another method of laser desorption/ionization. In one example of MALDI, the sample to be tested may be mixed with matrix and deposited directly on a MALDI chip. Depending on the sample being tested, an autoantibody may be first captured with bio-specific (for example, its corresponding antigen) or chromatographic materials coupled to a solid support such as a resin (for example, in a spin column). Specific affinity materials that may bind an autoantibody being detected. After purification on the affinity material, the auto-antibody under investigation is eluted and then detected by MALDI.

Analysis of autoantibodies by time-of-flight mass spectrometry generates a time-of-flight spectrum. The time-of-flight spectrum analysis typically represents the sum of signals from a number of pulses, which reduces dynamic range and noise. This time-of-flight data may be subjected to data processing using specialized software. Data processing may include TOF-to-M/Z transformation to generate a mass spectrum, baseline subtraction to eliminate instrument offsets and high frequency noise filtering to reduce high frequency noise.

Flow cytometry, for example, may be used to determine anti-GM-CSF autoantibody levels in a sample.

Phage display technology for expressing a recombinant antigen specific for anti— GM-CSF autoantibodies also can be used to determine the level of anti-GM-CSF autoantibody. Phage particles expressing the antigen specific for anti-GM-CSF autoantibody, or an antigen specific for anti-GM-CSF autoantibody, can be anchored, if desired, to a multiwell plate using an antibody such as an antiphage monoclonal antibody.

A variety of immunoassay formats including competitive and noncompetitive immunoassay formats may also be used (Self and Cook, “Advances in Immunoassay Technology,” Curr. Opin. Biotechnol. 7:60-65 (1996), which is incorporated by reference in its entirety). Immunoassays encompass capillary electrophoresis based immunoassays (CEIA) and can be automated, if desired. Immunoassays also may be used in conjunction with laser induced fluorescence (see e.g., Schmalzing et al., “Capillary Electrophoresis Based Immunoassays: A Critical Review,” Electrophoresis 18:2184-93 (1997) and Bao, J., “Capillary Electrophoretic Immunoassays,” Chromatogr. B. Biomed. Sci. 699:463-80 (1997), both of which are hereby incorporated by reference in their entirety). Liposome immunoassays, such as flow-injection liposome immunoassays and liposome immunosensors, also can be used to determine anti-GM-CSF autoantibody concentration.

Immunoassays, such as enzyme-linked immunosorbent assays (ELISAs), can be particularly useful. An ELISA, for example, can be useful for determining whether a sample is positive for anti-GM-CSF autoantibodies or for determining the anti-GM-CSF autoantibody level in a sample. An enzyme such as horseradish peroxidase (HRP), alkaline phosphatase (AP), or urease can be linked to a secondary antibody selective for anti-GM-CSF autoantibody, or to a secondary autoantibody selective for anti-GM-CSF autoantibody for use in the methods and compositions provided herein. A horseradish-peroxidase detection system can be used, for example, with the chromogenic substrate tetramethylbenzidine (TMB), which yields a soluble product in the presence of hydrogen peroxide. An alkaline phosphatase detection system can be used with the chromogenic substrate p- nitrophenyl phosphate, for example, which yields a soluble product readily detectable at a wavelength such as 405 nm. Similarly, a β-galactosidase detection system can be used with the chromogenic substrate o-nitrophenyl- -D-galactopyranoside (ONPG), which yields a soluble product detectable at 410 nm, or a urease detection system can be used with a substrate such as urea-bromocresol purple (Sigma Immunochemicals, St. Louis, Mo.). A useful secondary antibody linked to an enzyme can be obtained from a number of commercial sources; goat F(ab').sub.2 anti-human IgG-alkaline phosphatase, for example, can be purchased from Jackson Immuno-Research (West Grove, Pa.). In one embodiment, the measuring of the present aspect is conducted by enzyme-linked immunosorbent assay (ELISA), flow cytometry-based assay, and/or multiplex assay.

A radioimmunoassay also can be useful for determining the level of anti-GM-CSF autoantibodies in a sample. A radioimmunoassay using, for example, an iodine labeled secondary antibody (Harlow and Lane, ANTIBODIES A LABORATORY MANUAL, Cold Spring Harbor Laboratory: New York, 1988, which is incorporated herein by reference) is encompassed within the methods and compositions provided herein.

A secondary antibody labeled with a chemiluminescent marker also can be useful in the methods and compositions provided herein. A chemiluminescent secondary antibody is convenient for sensitive, non-radioactive detection of anti-GM-CSF autoantibodies and can be obtained commercially from various sources.

In addition, a detectable reagent labeled with a fluorochrome can be useful in the methods and compositions provided herein for determining the levels of anti-GM-CSF autoantibody in a sample. Appropriate fluorochromes include, for example, DAPI, fluorescein, Hoechst. 33258, R-phycocyanin, B-phycoerythrin, R-phycoerythrin, rhodamine, Texas red, or lissamine. A particularly useful fluorochrome is fluorescein or rhodamine. Secondary antibodies linked to fluorochromes can be obtained commercially.

A signal from the detectable reagent can be analyzed, for example, using a spectrophotometer to detect color from a chromogenic substrate; a radiation counter to detect radiation, such as a gamma counter for detection of iodine¹²⁵, or a fluorometer to detect fluorescence in the presence of light of a certain wavelength. For detection of enzyme-linked reagents, a quantitative analysis of the amount of anti-GM-CSF autoantibody can be made using a spectrophotometer such as an EMAX Microplate Reader (Molecular Devices, Menlo Park, Calif.) in accordance with the manufacturer's instructions. If desired, the assays can be automated or performed robotically, and the signal from multiple samples can be detected simultaneously.

Immunoassays using a secondary antibody selective for anti-GM-CSF autoantibodies are particularly useful in the methods and compositions provided herein.

Some embodiments of the methods and compositions provided herein can include measuring the level of anti-GM-CSF autoantibodies in a sample using a microarray (see, e.g., Price et al., “Protein Microarray Analysis Reveals BAFF-binding Autoantibodies in Systemic Lupus Erythematosus,” J. Clin. Invest. 123:5135-5145 (2013), which is hereby incorporated by reference in its entirety). In some embodiments, a microarray can include a nitrocellulose surface microarray platform containing GM-CSF. The GM-CSF can be printed on to a nitrocellulose-surface glass slides using a robotic microarrayer and software in replicates and across a range of concentrations. The array may be blocked in a protein solution, rinsed, and sample added comprising a primary anti-GM-CSF autoantibody. The array may be incubated, rinsed, and a fluorescently conjugated secondary antibody specific for the Fc region of the primary antibody probe.

Some embodiments of the methods and compositions provided herein can include measuring the level of anti-GM-CSF autoantibodies in a sample using particle-based technologies (see, e.g., Rosen et al., “Anti-GM-CSF Autoantibodies in Patients With Cryptococcal Meningitis,” J. Immunol. 190:3959-3966 (2013) and Ding et al.,“Determination of Human Anticytokine Autoantibody Profiles Using a Particle-Based Approach,” J. Clin. Immunol. 32:238-245 (2012), which are hereby incorporated by reference in their entirety). In some embodiments, fluorescing magnetic beads are conjugated to GM-CSF and beads are combined and incubated for with subject or control plasma, washed, and incubated with biotinylated mouse anti-human total IgG, as well as IgG subclasses, and IgA, IgM, and IgE (Sigma). Beads may be washed again and incubated with Streptavidin-PE (Bio-Rad) before being run in a multiplex assay on the Bio-Plex (Bio-Rad) instrument. Fluorescence intensity for each bead type is plotted as a function of Ab titer. See for example WO/2014/186416, which is hereby incorporated by reference in its entirety. In one embodiment, the anti-GM-CSF autoantibodies are selected from the group consisting of anti-GM-CSF IgA, anti-GM-CSF IgM, anti-GM-CSF IgG, anti-GM-CSF IgG1, anti-GM-CSF IgG2, anti-GM-CSF IgG3, and anti-GM-CSF IgG4.

Data generated by desorption and detection of autoantibodies can be analyzed with the use of a programmable digital computer. The computer program may analyze data to indicate the number of autoantibodies detected, and optionally the strength of the signal and the determined molecular mass for each autoantibody detected. Data analysis can include steps of determining signal strength of an autoantibody and removing data deviating from a predetermined statistical distribution. For example, the observed peaks can be normalized, by calculating the height of each peak relative to some reference. The computer can transform the resulting data into various formats for display. The standard spectrum can be displayed, and in one format the peak height and mass information are retained from the spectrum view, yielding a cleaner image and enabling autoantibodies with nearly identical molecular weights to be more easily seen. Using any of these formats, one can determine whether a particular autoantibody is present in a sample.

Analysis generally involves the identification of peaks in the spectrum that represent signal from an autoantibody. Peak selection may be done visually, but commercial software can be used to automate the detection of peaks. In general, this software functions by identifying signals having a signal-to-noise ratio above a selected threshold and labelling the mass of the peak at the centroid of the peak signal. In one example, many spectra are compared to identify identical peaks present in some selected percentage of the mass spectra. One version of this software clusters all peaks appearing in the various spectra within a defined mass range, and assigns a mass (M/Z) to all the peaks that are near the mid-point of the mass (M/Z) cluster.

Software used to analyze the data can include code that applies an algorithm to the analysis of the signal to determine whether the signal represents a peak in a signal that corresponds to an autoantibody under investigation. The software also can subject the data regarding observed autoantibody peaks to analysis, to determine whether an autoantibody peak or combination of autoantibody peaks is present that indicates the status of the particular clinical parameter under examination. Parameters of analysis include, for example, the presence or absence of one or more peaks, the shape of a peak or group of peaks, the height of one or more peaks, the log of the height of one or more peaks, and other arithmetic manipulations of peak height data.

Another technique for detecting the presence of an autoantibody involves the versatile fibre-based surface plasmon resonance (VeSPR) biosensor, as described in WO 2011/113085, which is hereby incorporated by reference in its entirety. Traditional SPR is a well-established method for label-free bio-sensing that relies on the excitation of free electrons at the interface between a dielectric substrate and a thin metal coating. The condition under which the incoming light couples into the plasmonic wave depends on the incidence angle and the wavelength of the incoming light as well as the physical properties (dielectric constant/refractive index) of the sensor itself and the surrounding environment. For this reason, SPR is sensitive to even small variations in the density (refractive index) in the close vicinity of the sensor, and does not require the use of fluorescent labels. The small variation of refractive index induced by the binding biomolecules such as autoantibodies onto the sensor surface, can be measured by monitoring the coupling conditions via either the incidence angle or the wavelength of the incoming light. Existing SPR systems may use the Krestchmann prism configuration where one side of the prism is coated with a metal such as gold or silver that can support a plasmonic wave. Alternative SPR architectures have been developed based on optical fibres with the metallic coating deposited around a short section of the fibre. This approach reduces the complexity and cost of such sensors, opening a pathway to distinctive applications such as dip sensing. The material at the sensor surface may be probed by monitoring the wavelength within a broad spectrum that is absorbed due to coupling to the surface plasmon. A powerful variant of an optical-fibre based SPR sensor, known as VeSPR, has also been developed.

An autoantibody can be detected by use of an agent that binds/interacts with an autoantibody in an indirect manner. With reference to the antibody-based detection methods described above, binding of a primary antibody specific for the autoantibody under investigation can be detected through use of a secondary antibody or reagent to the primary antibody. In effect, it is the binding or interaction of the secondary antibody or reagent with the primary antibody that is detected. The secondary antibody or reagent can be detected using the aforementioned methods.

In some instances it may be advantageous to detect the presence of an autoantibody by using an intermediary ligand that has binding affinity for the antigen or for the autoantibody if present in the sample, for example reactivity to the Fc region of the autoantibody or having reactivity to a region of the antigen different to the binding region of the autoantibody. The intermediary agent may be linked to a detectable label or marker molecule as described herein. The ligand may be an antibody which may thus be termed a secondary antibody. The antigen-autoantibody may be contacted with the labelled ligand or secondary antibody under conditions effective and for a period of time sufficient to allow the formation of secondary immune complexes. The secondary immune complexes may be washed to remove any unbound labelled ligand or secondary antibody, and the remaining label in the secondary immune complex may then be detected.

In the subject, the presence of the one or more anti-GM-CSF autoantibodies disclosed herein may be detected directly in the subject, or in an alternative embodiment, their presence may be detected in a sample obtained from a subject. The sample obtained from the subject that is analyzed by the methods of the present disclosure may have previously been obtained from the subject, and, for example, has been stored in an appropriate repository. In this instance, the sample would have been obtained from the subject in isolation of, and therefore separate to, the methods of the present disclosure.

In one embodiment, the method is performed in a subject having a preexisting condition or, alternatively, may be performed in a subject having no preexisting condition. The method may also be performed on a subject who has been previously treated for IBD, CD, and/or UC. In one embodiment, the sample is selected from the group consisting of whole blood, serum, urine, and nasal excretion.

In one embodiment, the method further includes detecting the presence or absence of additional markers. Examples of additional markers include but are not limited to anti-pANCA, ASCA, anti-CBir1 (flagellin), anti-OmpC (E. coli membrane), anti-A4 Fla2, and anti-FlaX.

In one embodiment, the presence of anti-GM-CSF autoantibodies in the subject correlates with an increased severity of Crohn's Disease as compared to the level of anti-GM-CSF autoantibodies in a reference sample. A reference sample may be obtained from a control subject, wherein a control subject does not have IBD and/or Crohn's Disease. Alternatively, a reference sample may be obtained from the subject before the subject is treated for IBD and/or Crohn's Disease. In yet another embodiment, the reference sample is from a subject that has been successfully treated for IBD and/or Crohn's Disease. In one embodiment, the reference subject has no anti-GM-CSF autoantibodies.

In some embodiments, a level of anti-GM-CSF autoantibodies in a sample from a subject having IBD and/or CD can be compared to the level of anti-GM-CSF autoantibodies in a sample obtained from the subject at a prior time, or in a sample obtained from another subject without IBD or without CD. In one embodiment, a sample obtained from the subject at a prior time can include a sample obtained at least about 1 day, at least about 2 days, at least about 5 days, at least about 10 days, at least about 30 days, at least about 60 days, at least about 75 days, at least about 100 days, at least about 200 days, at least about 1 year, at least about 2 years, at least about 3 years, at least about 4 years, at least about 5 years, at least about 6 years, at least about 7 years, at least about 8 years, at least about 9 years, at least about 10 years, or more, prior to the detection of presence or absence of anti-GM-CSF autoantibodies. In one embodiment, anti-GM-CSF autoantibodies identify severe forms of CD.

In some embodiments, the level of anti-GM-CSF autoantibodies in a sample, the relative change in the level of anti-GM-CSF autoantibodies in a sample, and/or an elevated IBD risk and/or an elevated CD risk in a subject can be provided to a third party. A party can include, for example, a health care provider such as a physician. In one embodiment, the third party can evaluate IBD risk and/or CD risk in a subject, select a treatment for the subject with the elevated IBD risk and/or CD risk, and/or administer a treatment. In one embodiment, the level of anti-GM-CSF autoantibodies in a sample, the relative change in the level of anti-GM-CSF autoantibodies in a reference sample, and/or an elevated IBD risk and/or CD risk of a subject can be provided to an automated system. In one such embodiment, an IBD risk for a subject having or at risk of having an IBD can be evaluated, and/or a treatment selected for the subject with the elevated IBD risk. In another embodiment, a CD risk for a subject having or at risk of having CD can be evaluated, and/or a treatment selected for the subject with the elevated CD risk. In one embodiment, IBD-associated anti-GM-CSF autoantibodies are distinct and alter GM-CSF receptor signaling. In one embodiment, CD-associated anti-GM-CSF autoantibodies block GM-CSF receptor signaling. In one embodiment, anti-GM-CSF autoantibodies recognize structural epitopes. In another embodiment, post-translational modified GM-CSF is targeted by anti-GM-CSF autoantibodies in CD. In one embodiment, post-translational modified GM-CSF is targeted by anti-GM-CSF autoantibodies in CD. In yet another embodiment, anti-GM-CSF autoantibodies are a predictive biomarker for Crohn's Disease.

In one embodiment, the method further comprises administering one or more additional treatments. Examples of additional treatments include any standard treatment known by those skilled in the art for the treatment of IBD, CD, and/or UC. In one embodiment, the additional treatment is, for example, anti-plasma cell treatment and/or anti-idiotype treatment.

Another aspect of the present disclosure relates to a method of preventing or treating Crohn's Disease and/or a condition resulting from Crohn's Disease in a subject. The method includes selecting a subject having or at risk of having Crohn's Disease and administering a recombinant Granulocyte Macrophage-Colony Stimulating Factor (GM-CSF) protein to the selected subject under conditions effective to prevent or treat Crohn's Disease and/or a condition resulting from Crohn's Disease in the subject.

This aspect of the present disclosure is carried out in accordance with previously described aspects of the disclosure.

Administration of the compositions described herein may be completed by any suitable route. In one embodiment, the treatment comprises administering said recombinant GM-CSF to a subject orally, by inhalation, by intranasal instillation, topically, transdermally, intradermally, parenterally, subcutaneously, intravenous injection, intra-arterial injection, intramuscular injection, intrapleurally, intraperitoneally, intrathecally, or by application to a mucous membrane.

In another embodiment, the method further comprises repeating said administering the recombinant GM-CSF.

In accordance with the present disclosure, approximately 30% of IBD patients may have anti-GM-CSF (i.e., CSF2) titers. Anti-GM-CSF (i.e., CSF2) titers may, in one embodiment, be correlated with Ileal CD and increased disease severity. This characterization may, in one embodiment, reveal CD-specific isotypes and may, in precede the onset of active disease by years. The anti-CSF2 antibodies may also recognize post-translational modifications (PTM).

In accordance with the present disclosure, anti-GM-CSF autoantibodies are a predictive marker of severe Crohn's Disease. IBD-associated anti-GM-CSF autoantibodies are distinct and alter GM-CSFR signaling. Moreover, anti-GM-CSF autoantibodies identify severe forms of CD and CD-associated anti-GM-CSF autoantibodies block GM-CSFR signaling. Anti— GM-CSF autoantibodies recognize structural epitopes. Post-translationally modified GM-CSF, in one embodiment, is targeted by anti-GM-CSF autoantibodies in CD. Anti-GM-CSF autoantibodies are, in accordance with the present disclosure, a predictive biomarker for CD in one embodiment.

As described herein, glycosylation sites on human GM-CSF (or CSF2), for example, include S22, S24, T27, S26, N44, and/or N54. Glycosylation in accordance with the present disclosure is important for protein structure, function, and stability (half-life). In one embodiment, stable cell lines expressing human GM-CSF (i.e., CSF2) deficient in glycosylation sites may be produced. STATS phosphorylation in accordance with the present disclosure may be stimulated in U937 cells with recombinant human GM-CSF (i.e., CSF2). Recombinant human GM-CSF (i.e., CSF2) deficient in glycosylation sites is biologically active and HIS-tag purification yields recombinant human GM-CSF (i.e., CSF2) from stable HEK293 clones lacking one or all glycosylations in accordance with the present disclosure. Purification of recombinant human GM-CSF (i.e., CSF2) deficient in glycosylation in accordance with the present disclosure does not alter biologically activity. Patient serum contains anti-GM-CSF autoantibodies against wild type variant of GM-CSF, specifically recognizing variants with anti-IgA antibodies against the wild type form and IgM and IgG antibodies recognizing the GM-CSF variants.

In accordance with the present disclosure, anti-GM-CSF (i.e., anti-CSF2) titers are elevated in a subgroup of CD patients, which correlates with Ileal CD and increased disease severity. In accordance with the present disclosure anti-GM-CSF (i.e., anti-CSF2) antibodies have CD-specific isotypes and the presence of these anti-GM-CSF (i.e., anti-CSF2) antibodies precede the onset of active disease by years. The anti-GM-CSF (i.e., anti-CSF2) antibodies in accordance with the present disclosure recognize post-translational modifications (PTM) and the removal of PTM may rescue the effect of anti-GM-CSF (i.e., anti-CSF2) antibodies. Accordingly, in one embodiment, genetic engineering of GM-CSF (i.e., CSF2) to avoid recognition by anti-GM-CSF (i.e., CSF2) antibodies may be useful for a personalized therapy of CD. In another embodiment, the recombinant GM-CSF (i.e., CSF2) may be produced and designed to lack specific glycosylation sites. In another embodiment, PTM-specific anti-GM-CSF (i.e., anti-CSF2) ELISA as diagnostic assay may be useful for the subclassification of CD patients. In another embodiment, use of recombinant GM-CSF (i.e., CSF2) variants as therapeutic for sero-positive anti-GM-CSF (i.e., CSF2) patients may be used.

For purposes of this and other aspects of the disclosure, the target “subject” encompasses any vertebrate, such as an animal, preferably a mammal, more preferably a human. In the context of administering a composition of the disclosure for purposes of preventing and/or treating IBD or Crohn's Disease and/or a condition resulting from IBD or Crohn's Disease in a subject comprising in a subject, the target subject encompasses any subject that has or is at risk of having IBD or Crohn's Disease. Particularly susceptible subjects include adults and elderly adults. However, any infant, juvenile, adult, or elderly adult that has or is at risk of having IBD or Crohn's Disease can be treated in accordance with the methods of the present disclosure. In one embodiment, the subject is an infant, a juvenile, or an adult.

As used herein, the phrase “therapeutically effective amount” means an amount of compound or composition that elicits the biological or medicinal response that is being sought in a tissue, system, animal, individual or human by a researcher, veterinarian, medical doctor, or other clinician. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size, and sex of the subject. Optimal amounts can also be determined based on monitoring of the subject's response to treatment. The term “treatment” or “treat” may include effective inhibition, suppression or cessation of IBD or CD symptoms so as to prevent or delay the onset, retard the progression, or ameliorate the symptoms of the IBD and/or CD.

One goal of treatment is the amelioration, either partial or complete, either temporary or permanent, of patient symptoms, including inflammation of the mucosa, extraintestinal manifestations of the disease, epithelial damage, and/or any early markers of IBD and any early markers of CD. Any amelioration is considered successful treatment. This is especially true as amelioration of some magnitude may allow reduction of other medical or surgical treatment which may be more toxic or invasive to the patient. Extraintestinal disease manifestations include those of the liver, bile duct, eyes, and skin. Another goal of the treatment is to maintain a lack of excess intestinal inflammation in persons who have already achieved remission. In one embodiment, the IBD or Crohn's Disease and/or the condition resulting from IBD or Crohn's Disease is prevented. In another embodiment, the IBD or Crohn's Disease and/or the condition resulting from IBD or Crohn's Disease is treated.

As used herein a sample may include any sample obtained from a living system or subject, including, for example, blood, serum, and/or tissue. In one embodiment, a sample is obtained through sampling by minimally invasive or non-invasive approaches (for example, by urine collection, stool collection, blood drawing, needle aspiration, and other procedures involving minimal risk, discomfort, or effort). Alternatively, samples may be gaseous (for example, breath that has been exhaled) or liquid fluid. Liquid samples may include, for example, urine, blood, serum, interstitial fluid, edema fluid, saliva, lacrimal fluid, inflammatory exudates, synovial fluid, abscess, empyema or other infected fluid, cerebrospinal fluid, sweat, pulmonary secretions (sputum), seminal fluid, feces, bile, intestinal secretions, nasal excretions, and other liquids. Samples may also include a clinical sample such as serum, plasma, other biological fluid, or tissue samples, and also includes cells in culture, cell supernatants and cell lysates. In one embodiment, the sample is selected from the group consisting of whole blood, serum, urine, and nasal excretion. Samples may be in vivo or ex vivo.

In one embodiment, the method includes administering one or more additional agents which prevent or treat Crohn's Disease and/or a condition resulting from Crohn's Disease in the subject.

Examples of additional agents that may be administered include but are not limited to corticosteroids, used primarily for treatment of moderate to severe flares of IBDs, such as CD, such as, for example, prednisone and budesonide; 5-aminosalicylates, useful in the treatment of mild-to-moderate IBDs, such as CD, examples which include 5-aminosalicylic acid (mesalazine), and sulfasalazine; Azathioprine and 6-mercaptopurine (6-MP) for maintenance therapy of IBDs, such as CD; TNF inhibitors useful for treating various severities of IBDs, such as CD, examples include infliximab, adalimumab, natalizumab; methotrexate; and surgery.

As used herein, the term “simultaneous” therapeutic use refers to the administration of at least one additional agent beyond the recombinant Granulocyte Macrophage-Colony Stimulating Factor (GM-CSF), for example, agents administered before, during, or after the recombinant GM-CSF, optionally, by the same route and at the same time or at substantially the same time. As used herein, the term “separate” therapeutic use refers to an administration of at least one additional agent beyond the recombinant GM-CSF, for example, agents administered before, during, or after administration of a recombinant GM-CSF, at the same time or at substantially the same time by different routes. As used herein, the term “sequential” therapeutic use refers to administration of at least one additional agent beyond the recombinant GM-CSF, for example, agents administered before, during, or after administration of the recombinant GM-CSF, at different times, the administration route being identical or different. More particularly, sequential use refers to the whole administration of the additional agent before administration of recombinant GM-CSF. It is thus possible to administer the additional agent over several minutes, hours, or days before applying the recombinant GM-CSF. In one embodiment, the additional agent is administered before, during, or after the recombinant GM-CSF.

Another aspect of the present disclosure relates to a method for diagnosing and/or predicting severity of and/or treating Crohn's Disease. The method includes detecting a glycoprofile of GM-CSF in a sample, and diagnosing Crohn's Disease and/or predicting the severity of Crohn's Disease based on said detecting.

This aspect of the present disclosure is carried out in accordance with previously described aspects of the disclosure.

In one embodiment, the method further includes administering a recombinant Granulocyte Macrophage-Colony Stimulating Factor (GM-CSF) protein to the diagnosed subject.

In one embodiment, when the sample provides a higher expression of mannose in GM-CSF compared to a reference sample, Crohn's Disease is diagnosed and/or the severity of Crohn's Disease is predicted. In one embodiment, the mannose is one or more mannosylated N-glycans.

In one embodiment, when the sample provides a decrease in presence of one or more core fucose in GM-CSF compared to a reference sample, Crohn's Disease is diagnosed and/or the severity of Crohn's Disease is predicted.

In the following description, reference is made to the accompanying drawings that form a part hereof, and in which is shown by way of illustration specific embodiments which may be practiced. These embodiments are described in detail to enable those skilled in the art to practice the disclosure, and it is to be understood that other embodiments may be utilized and that structural, logical and electrical changes may be made without departing from the scope of the present disclosure. The following description of example embodiments is, therefore, not to be taken in a limited sense.

The present disclosure may be further illustrated by reference to the following examples.

### EXAMPLES

The following examples are intended to illustrate, but by no means are intended to limit, the scope of the present disclosure as set forth in the appended claims.

### Example 1—Materials and Methods

Human specimen. Non-involved intestinal resection and involved intestinal resection samples were obtained from patients undergoing ileal resection surgery at the Mount Sinai Medical Center (New York, NY) after obtaining informed consent. Serological analysis of these patients was not performed. All protocols were reviewed and approved by the Institutional Review Board (IRB) at the Icahn School of Medicine at Mount Sinai (IRB 08-1236). Stored pre-diagnosis serum samples were obtained from the Department of Defense Serum Repository, Silver Spring, MD, USA. 220 CD, 200 UC, and 200 healthy controls (HC) samples were provided, sampled at two to three time points prior to diagnosis of disease and one time point post diagnosis of disease.

Sample collection. CD patients were consented under IRB HSM 13-00998. Resection samples from patients described above were taken to undergo clearance by a pathologist. Following clearance, tissues were placed in complete RPMI media (Corning) on ice prior to processing. Patients diagnosed with UC or malignancies were excluded from the analysis by flow cytometry and mass cytometry.

Isolation of PBMC. Buffy coats were obtained from the New York City Blood Center. PBMCs were collected via a Ficoll gradient (GE Healthcare). PBMCs were washed and resuspended in complete RPMI media to create a single cell suspension. Cells were counted with a hemocytometer. Leukocytes were either used for consecutive experiments or further used for the enrichment of monocytes using anti-CD14-beads (Miltenyi) and magnetic enrichment. Purity of monocytes was >98%.

Isolation of lamina propria leukocytes. For the isolation of lamina propria leukocytes, freshly removed ileal resections were washed in ice-cold PBS, mucus was scraped off and epithelial cells were removed by incubating the tissues in HB SS free of calcium and magnesium, supplemented with EDTA (5 mM) and HEPES (10 mM) for 20-30 minutes at 37° C. at 100rpm. Samples were vortex, supernatant was removed and the remaining tissue washed in HB SS containing calcium and magnesium (Ca²⁺Mg²⁺). Fibrotic tissue was removed and non-fibrotic mucosa was minced and digested using Collagenase IV and DNase I (both Sigma) in HB SS (Ca²⁺Mg²⁺) 2% FBS for 30 minutes at 37° C. at 100rpm agitation. The cell suspension was filtered and life leukocytes were enriched on a 80%:40% Percoll (GE Healthcare) gradient. The interphase was harvested, washed extensively with FACS buffer (PBS, 5 mM EDTA and 2% FBS). Cells were subsequently used for FACS analysis or ex vivo GM-CSF stimulation.

Flow Cytometry. Aldefluor staining: freshly isolated PBMCs or lamina propria leukocytes were washed twice in PBS and stained following the manufacturer protocol. Following Aldefluor staining, cells were washed in PBS and processed for surface staining.

Surface staining. Fc-receptors were blocked using Fc-block reagent (BD), following a 20 minute surface staining with directly conjugated monoclonal antibodies. The following antibodies were used and purchased from BD, R+D, Biolegend and Miltenyi: anti-human CD45 Pacific Orange, anti-human HLA-DR APC-Cy7, anti-human CD11 c Pe-Cy7, anti-human CD14 APC, anti-human CD1c PerCP-Cy5.5, anti-human CD141 Pe, anti-human CD127 FITC, anti-human CD117 Pe-Cy7, anti-NKp44 APC, anti-NKp44 Pe, anti-CD161 PerCP-Cy5.5, anti-CD3 e450, anti-CD19 e450, anti-RORγt APC, anti-CD69 Pe. Following surface staining, cells were fix using the FOXP3 staining kit to stain for RORγt.

Intracellular cytokine staining. For intracellular cytokine staining of GM-CSF and IFN-γ, cells were re-suspended in complete RPMI (Corning), 10% FBS (Life Technologies), 1% non-essential amino acids (Corning), 1% sodium-pyruvate (Corning), 1% L-glutamine (Corning), 1% penicillin-streptomycin (Life Technologies) and 1% HEPES buffer (Corning). Media was further supplemented with Brefeldin A (for GM-CSF) or Brefeldin A, 1. PMA (Sigma) and Ionomycin (Sigma) (for IFN-γ). Cells were incubated for 4 hours at 37° C. and 5% CO2 prior to surface staining (as described above). Post surface staining, cells were fixed for 30 minutes in Cytofix/CytoPerm (BD). Cells were washed using PBS containing 2% FBS, 5 mM EDTA (FACS buffer) and 0.5% Saponin (Sigma). Anti-human GM-CSF and anti-human IFN-γ staining was performed in FACS buffer containing 0.5% Saponin for 30 minutes at 4° C. in the dark. Cells were washed and samples were analyzed on a BD LSR Fortessa II.

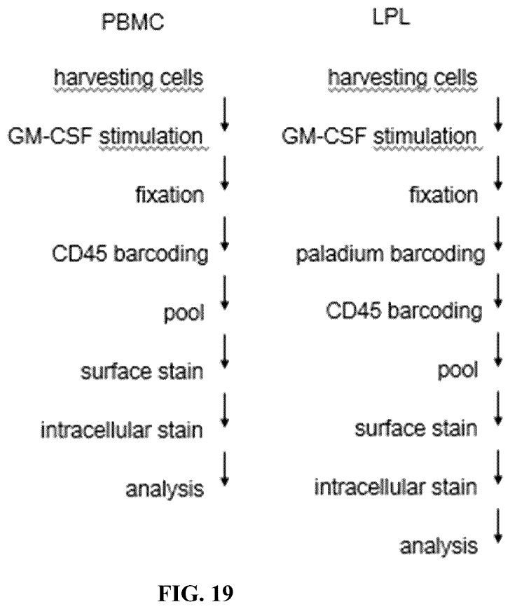

Phospho STAT5 staining. STAT5 phosphorylation was assessed using the BD PhosFlow Protocol for human PBMCs using PermBuffer II. Intracellular staining of pSTAT5 was performed using anti-human pSTAT5-Alexa647. FIG. 19 shows a scheme demonstrating the workflow for pSTAT5 staining in samples.

Mass Cytometry. CyTOF data was visualized using viSNE (Amir et al., “viSNE Enables Visualization of High Dimensional Single-cell Data and Reveals Phenotypic Heterogeneity of Leukemia” Nat. Biotechnol. 31:545-52 (2013), which is hereby incorporated by reference in its entirety), a dimensionality reduction method which uses the Barnes-Hut acceleration of the t-SNE algorithm (van der Maaten, “Accelerating t-SNE Using Tree-Based Algorithms,” Journal of Machine Learning Research 15(93):3221-3245 (2014) and van der Maaten et al., “Visualizing Data using t-SNE,” Journal of Machine Learning Research 9:2579-2605 (2008), which are hereby incorporated by reference in their entirety). viSNE was implemented using Cytobank (Chen et al., “Cytobank: Providing an Analytics Platform for Community Cytometry Data Analysis and Collaboration,” Curr. Top. Microbiol. Immunol. 377:127-57 (2014), which is hereby incorporated by reference in its entirety).

ELISA. For anti-GM-CSF ELISAs, plates were coated with recombinant human GM-CSF (Sargramostim), washed and blocked with TB ST/BSA. Wells were incubated with 10-of serum diluted in TBST followed by three washing steps. Anti-GM-CSF antibodies were detected by pan anti-human IgG HRP or isotype specific secondary antibodies. Substrate reaction was assessed using a plate reader at 550 nm.

Antibody-binding assay. GM-CSF was boiled in SDS containing buffer to generate denaturated GM-CSF. Denaturated GM-CSF was then used in anti-GM-CSF ELISAs. To assess the binding-strength of anti-GM-CSF antibodies in patient sera, GM-CSF coated plates were incubated with 10-50 μl of serum and washed with NaCl salt solutions of increasing concentrations (1-4M NaCl). Binding capacity was calculated as % of maximal binding.

Stripping of GM-CSF. GM-CSF was stripped using N-Glycosidase F, α2-3,6,8,9-Neuraminidase, Endo-α-N-acetylgalactosaminidase, β1,4-galactosidase and β-N-Acetylglucosaminidase (SIGMA) and used according to the manufacturers recommendations.

Native PAGE, SDS-PAGE/Western Blot. Recombinant and stripped GM-CSF (Sargramostim) (8 μg/well) were separated on 15% resolving native polyacrylamide gels. Gels were stained with Coomassie G Brilliant Blue to confirm stripping. Proteins were then transferred to nitrocellulose membranes and membranes were blocked with 5% Non-Fat Dry Milk in Tris-Buffer Saline 0.1% Tween-20 (TBST) at 4° C. Membranes were then incubated with serum samples (diluted 1:100 in blocking buffer). Bound anti-GM-CSF antibodies were detected using anti-Human IgG AP at 1:1000 in TB ST.

Cloning, mutagenesis, expression and purification of GM-CSF. A double-stranded DNA fragment of GM-CSF with Nhel and EcoRl RE sites and a C-terminal polyhistidine-tag (His-tag) were obtained from Integrated DNA Technologies and cloned into pIRESpuro2. Mutagenesis of putative glycosylation sites on GM-CSF were performed using primers designed with NEBaseChanger and the Q5® Site-Directed Mutagenesis Kit (New England Biolabs, Cat E0554S). Annealing temperatures were determined through gradient PCR assays. Bacterial colonies were picked, sequenced, and analyzed for correctness of sequences. Wild type and mutated GM-CSF was transfected into HEK293 cells. Stable clones were selected using puromycin selection. Cytokine secretion was validated using Flow cytometry and intracellular cytokine staining as well as ELISA. Cell culture supernatant of stable GM-CSF producing HEK293 cells were purified using Ni-columns. Purity was determined by western blot and Coomassie Brilliant Blue-stained polyacrylamide gels. Recombinant GM-CSF variants were tested for bioactivity on U937 cells using Phopho flow.

### Example 2—CD-Associated Anti-GM-CSF Autoantibodies are Distinct from those in PAP and UC

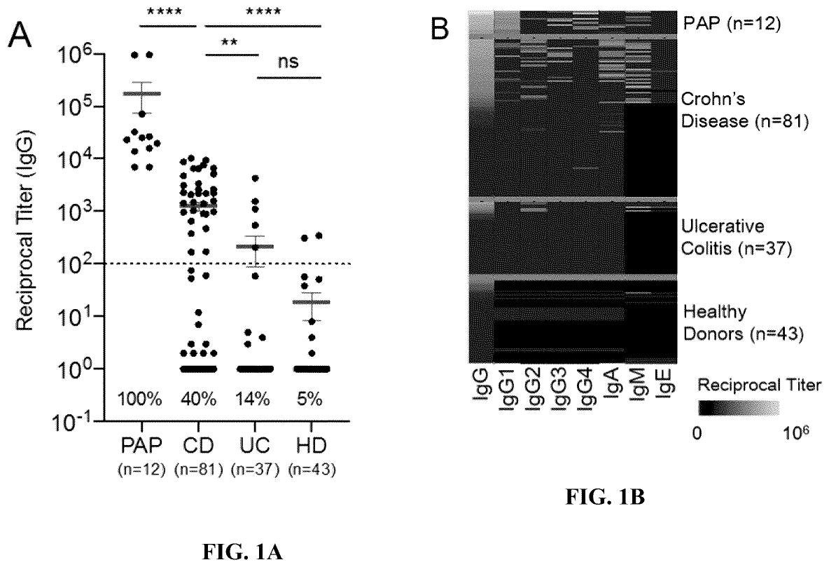

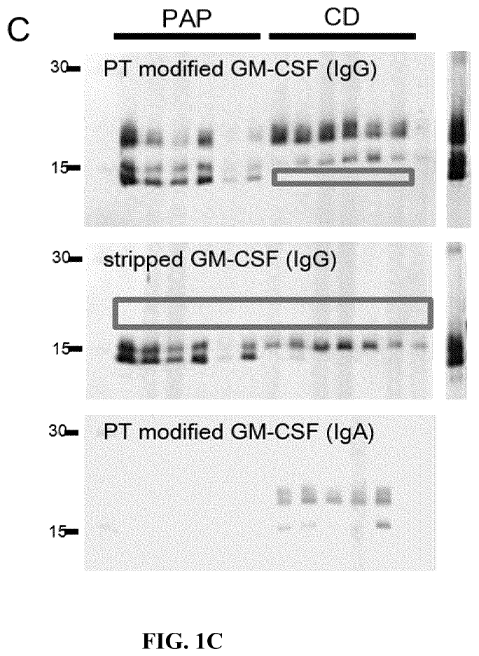

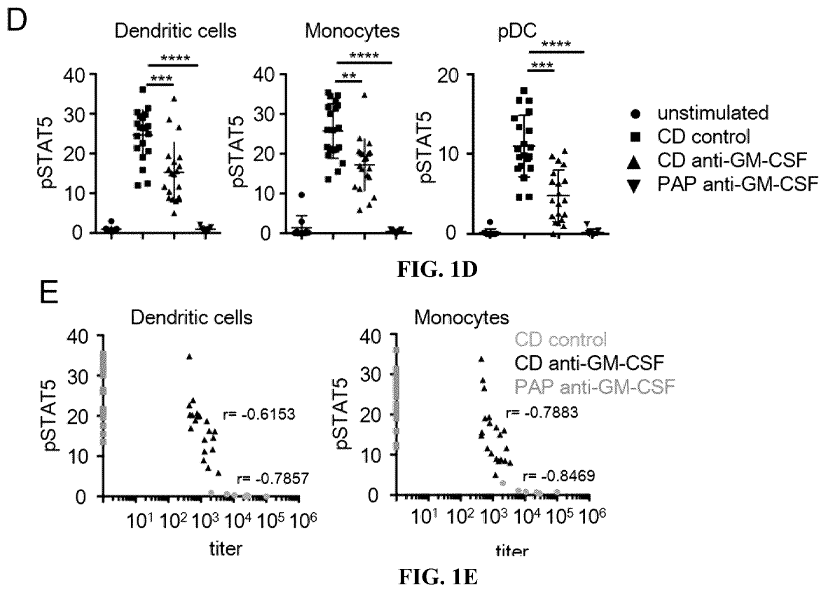

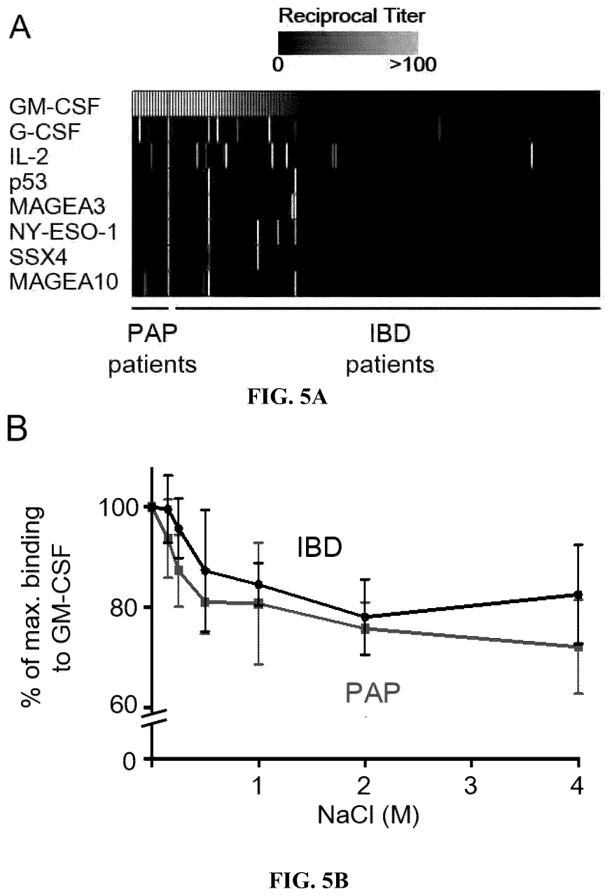

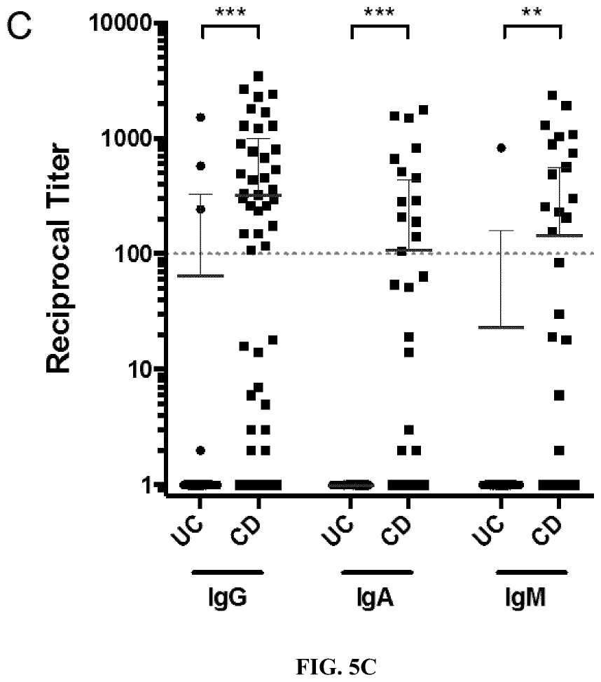

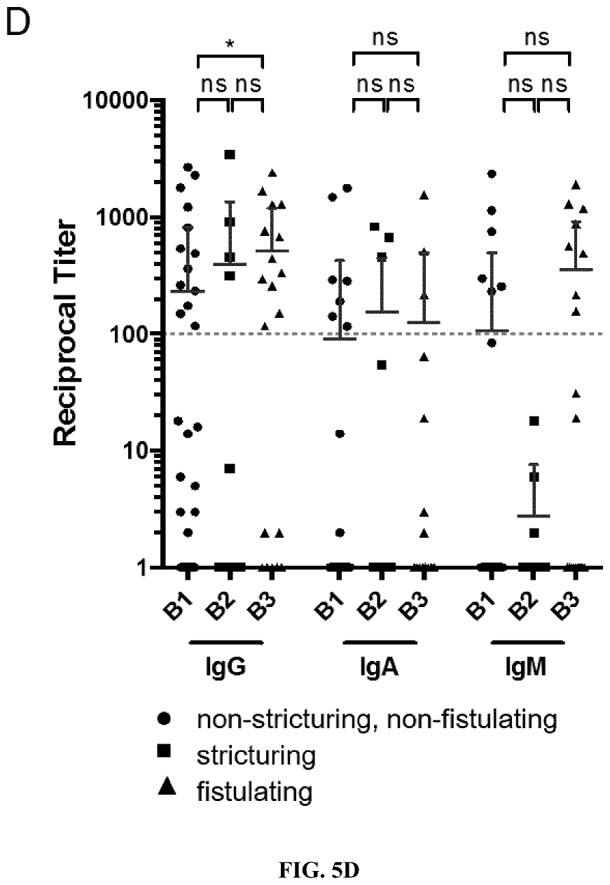

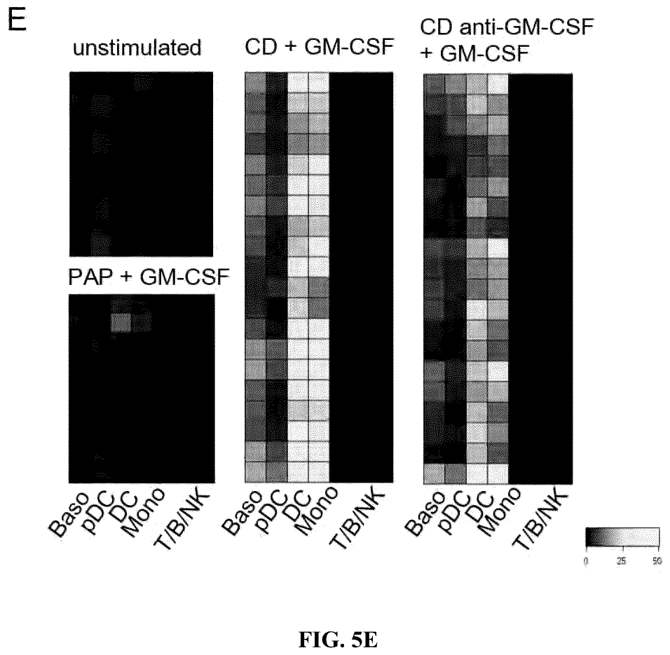

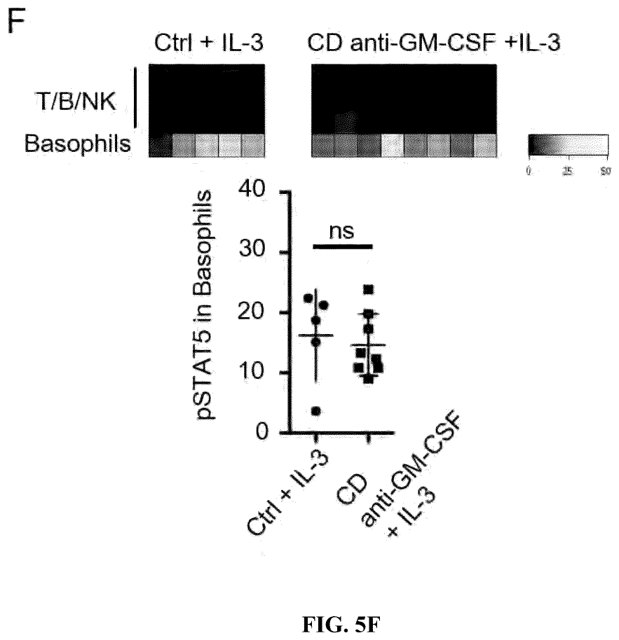

To assess the prevalence and characteristics of autoantibodies against GM-CSF in IBD, the presence of serum IgG titers were tested by ELISA using Sargramostim, a yeast-produced recombinant GM-CSF, in a cohort of patients with active CD (n=81) or UC (n=37), as well as in healthy donors (HD, n=43) and PAP patients (n=12) as controls. Among IBD patients, 40% of CD and 14% of UC patients displayed detectable levels (defined as titers greater than 1/100) of anti-GM-CSF IgG autoantibodies in their serum (FIG. 1A). Titers detected in CD but not UC patients were significantly higher compared to those observed in HD sera, though much lower than those detected in PAP patients (FIG. 1A). Importantly, sera from IBD patients with anti-GM-CSF autoantibodies rarely cross-reacted with other known autoantigens, emphasizing that these serum antibody responses were antigen-specific (FIG. 5A). Sera showing cross-reactivity to unrelated antigens, were excluded from further analysis. Even though anti-GM-CSF autoantibody titers were significantly different between PAP and IBD patients, adjusted titers demonstrated comparable, relative avidity in binding GM-CSF in the presence of increasing salt concentrations (FIG. 5B).

Considering the distinctly contained localization and anatomic features of the pathologies seen in PAP and CD, despite shared seroreactivity against GM-CSF, the isotypes and IgG subclasses of anti-GM-CSF autoantibodies were examined in IBD vs. PAP patients using specific secondary antibodies. While PAP-associated anti-GM-CSF autoantibodies were almost exclusively of IgG1 and IgG4 subclass—typically associated with Th1 and chronic exposure, these isotypes were virtually absent in IBD-associated anti-GM-CSF autoantibodies, which were instead significantly enriched in IgG2 and IgA- typically associated with Th2 and mucosal immunity. IgM and IgG3, but not IgE, were detectable in both PAP and IBD, with higher average IgM titers to GM-CSF in CD patients (FIG. 1B). Interestingly, anti-GM-CSF autoantibodies in IBD patients were not associated with sex or age, but were a specific marker for CD patients with ileal involvement and increased disease severity, confirming results reported in previous studies (FIG. 5C and 5D and Table 1). See Gathungu et al., “Granulocyte-macrophage Colony-Stimulating Factor Autoantibodies: A Marker of Aggressive Crohn's Disease,” Inflamatory Bowel Disease 19:1671-1680 (2013); Nylund et al., “Granulocyte Macrophage-Colony-Stimulating Factor Autoantibodies and Increased Intestinal Permeability in Crohn Disease,” Journal of Pediatric Gastroenterology and Nutricion 52:542-548 (2011); Jurickova et al., “Paediatric Crohn Disease Patients With Stricturing Behaviour Exhibit Ileal Granulocyte-Macrophage Colony-Stimulating Factor (GM-CSF) Autoantibody Production and Reduced Neutrophil Bacterial Killing and GM-CSF Bioactivity,” Clinical and Experimental Immunology 172:455-465 (2013); and Dabritz et al., “Granulocyte Macrophage Colony-Stimulating Factor Auto-Antibodies and Disease Relapse in Inflammatory Bowel Disease,” American Journal of Gastroenterology 108:1901-1910 (2013), all of which are hereby incorporated by reference in their entirety. Serological assessment of anti-GM-CSF autoantibodies thus allows the discrimination of a subgroup of CD patients amongst all IBD and PAP patients.

TABLE 1
Available Patient Information for Serum Samples used in FIGS. 1A-1E.

| | Crohn's disease | | | | Ulcerative colitis | | | |
|---|---|---|---|---|---|---|---|---|
| | | IgG | IgA | IgM | | IgG | IgA | IgM |
| | | positive | positive | positive | | positive | positive | positive |
| | Total | patients | patients | patients | Total | patients | patients | patients |
| Number of patients | 83 | 30 | 13 | 14 | 37 | 3 | 0 | 1 |
| Gender | | | | | | | NA | |
| Male | 43 | 20 | 8 | 10 | 17 | 1 | | 0 |
| Female | 40 | 10 | 5 | 4 | 20 | 2 | | 1 |
| Age, years | 42 | 40 | 46 | 35 | 38 | 27.5 | NA | 19 |
| (range) | (23-79) | (23-79) | (26-79) | (26-44) | (19-63) | (19-36) | | |
| Smoking | | | | | | | NA | |
| Yes | 17 | 3 | 2 | 1 | 12 | 2 | | 0 |
| No | 63 | 26 | 11 | 13 | 25 | 1 | | 1 |
| Unknown | 3 | 1 | 0 | 0 | 0 | 0 | | 0 |
| Disease behavior | | | | | NA | NA | NA | NA |
| B1 | 48 | 13 | 7 | 6 | | | | |
| B2 | 13 | 4 | 3 | 0 | | | | |
| B3 | 19 | 12 | 3 | 8 | | | | |
| Unknown | 3 | 1 | 0 | 0 | | | | |
| Heal involvement | | | | | NA | NA | NA | NA |
| Yes | 64 | 27 | 11 | 14 | | | | |
| No | 12 | 1 | 0 | 0 | | | | |
| Unknown | 7 | 2 | 2 | 0 | | | | |
| Colorectal involvement | | | | | NA | NA | NA | NA |
| Yes | 53 | 17 | 8 | 8 | | | | |
| No | 17 | 6 | 1 | 3 | | | | |
| Unknown | 13 | 7 | 4 | 3 | | | | |
| Anti-GM-CSF Ab titer | | | | | | | NA | |
| IgG | 317 | 875 | 1257 | 877 | 64 | 780 | | 576 |
| IgA | 106 | 289 | 655 | 431 | 0 | 0 | | 0 |
| IgM | 144 | 394 | 472 | 838 | 23 | 276 | | 82 |

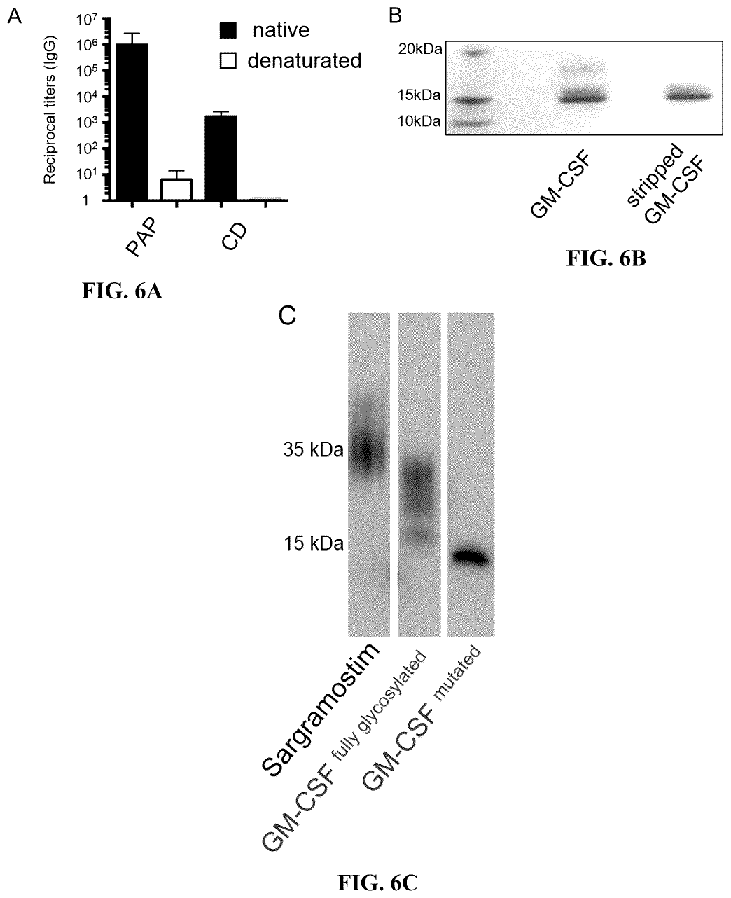

Another major difference between IBD and PAP seroreactivity to GM-CSF was found in the epitopes recognized. Peptide epitopes recognized in PAP patients have previously been described as located in the N-terminal region of the GM-CSF protein (Piccoli et al., “Neutralization and Clearance of GM-CSF by Autoantibodies in Pulmonary Alveolar Proteinosis,” Nature Communications 6:7375 (2015), which is hereby incorporated by reference in its entirety), but synthetic overlapping 20-mer linear peptides covering GM-CSF sequence failed to react with CD-associated anti-GM-CSF autoantibodies. Suspecting conformational epitopes were important, the binding of anti-GM-CSF autoantibodies on structurally intact or denaturated GM-CSF by ELISA was then assessed. Recognition of GM-CSF was significantly decreased for PAP sera and virtually absent for CD sera following denaturation, confirming the need for intact antigen (FIG. 6A). In contrast to PAP however, CD sera did not react with GM-CSF in the absence of post-translational modifications: only PAP sera had detectable titers in ELISA when using bacterially produced recombinant GM-CSF, while CD sera required a eukaryotic GM-CSF product in order to react. Analysis of yeast-produced recombinant GM-CSF protein (Sargramostim) using native and SDS-polyacrylamide gel electrophoresis (PAGE/SDS-PAGE) revealed three bands at ˜19.5 kDa, ˜16.5 kDa and ˜14.5 kDa (FIG. 6B). The highest band carried posttranslational sugar modifications that were lost when GM-CSF was enzymatically stripped (FIG. 6B). GM-CSF expressed recombinantly in HEK293 cells showed a comparable pattern that was lost when all putative glycosylation sites were genetically mutated to alanine (FIG. 6C). Posttranslational modifications of GM-CSF have previously been reported and may serve as ideal antibody-recognition sites. Miyajima et al., “Expression of Murine and Human Granulocyte-Macrophage Colony-Stimulating Factors in S. Cerevisiae: Mutagenesis of the Potential Glycosylation Sites,” The EMBO Journal 5:1193-1197 (1986), which is hereby incorporated by reference in its entirety. Using PAGE and western blot analysis, it was assessed whether sera from CD patients were able to react with the different forms of yeast-recombinant human GM-CSF (Sargramostim). While PAP-associated sera recognized all three bands of Sargramostim, CD-associated anti-GM-CSF autoantibodies exclusively bound the larger bands carrying posttranslational modifications, but not to the 14.5 kDa band corresponding to unmodified GM-CSF protein (FIG. 1C, FIGS. 6B and 6C). When Sargramostim was enzymatically stripped of sugars, seroreactivity of CD to the 19.5 kDa band was lost but still remained to the 16.5 kDa (FIG. 1C), while PAP sera also recognized the 14.5 kDa band. Piccoli et al., “Neutralization and Clearance of GM-CSF by Autoantibodies in Pulmonary Alveolar Proteinosis,” Nature Communications 6:7375 (2015), which is hereby incorporated by reference in its entirety. Similarly to IgG, anti-GM-CSF autoantibodies of the IgA isotype showed high specificity to posttranslational modifications and were exclusively found in serum from CD patients (FIG. 1C). It was also confirmed that the anti-GM-CSF specificity of CD-associated autoantibodies using recombinant human GM-CSF produced in human cells, indicating that the reactivity was not due to exclusive yeast-specific modifications. Together, these findings demonstrate that CD-associated anti-GM-CSF autoantibodies selectively recognize post-translational modifications on GM-CSF that require a structurally intact protein.

### Example 3—Neutralizing Capacity of CD-Associated Anti-GM-CSF Autoantibodies

The enriched abundance of anti-GM-CSF autoantibodies in CD patients (Table 1 and FIG. 1A), their strong binding to GM-CSF (FIG. 5B) as well as previous reports (Han et al., “Granulocyte-macrophage Colony-Stimulating Factor Autoantibodies in Murine Ileitis and Progressive Ileal Crohn's Disease,” Gastroenterology 136:1261-1271 (2009), which is hereby incorporated by reference in its entirety), support the hypothesis that anti-GM-CSF autoantibodies possess neutralizing capacities. To determine whether CD-associated anti-GM-CSF autoantibodies abrogate GM-CSFR signaling in monocytes, dendritic cells (DC) and plasmacytoid DC (pDC) within PBMCs from healthy donors, blood leukocytes were purified and stimulated with recombinant human GM-CSF (Sargramostim) ex vivo for 20 minutes in the presence of individual patient sera, either obtained from PAP patients (n=9), CD patients negative for anti-GM-CSF autoantibodies (n=20), or CD patients positive for anti-GM-CSF autoantibodies (n=20). Post stimulation, PBMCs were barcoded, pooled, surface stained, fixed, and intracellularly stained to determine the phosphorylation of STATS as readout for GM-CSFR activation within T cells, B cells, NK cells, monocytes, basophils, DC, and pDC by mass cytometry (Table 2).

Table 2 shows antibodies and isotope conjugates used in mass-cytometry analysis of peripheral blood mononucleated cells (PBMC) and lamina propria leukocytes (LPL).

TABLE 2
Antibodies and Isotope Conjugates for Mass-Cytometry Analysis

| A | | B | |
|---|---|---|---|
| PBMC | | LPL | |
| isotope | antigen | isotope | antigen |
| Pd102Di | Pd102Di | Rh103Di | viability |
| Rh103Di | Viability | In115Di | CD45_115 |
| Pd104Di | Pd104Di | La139Di | FceR1a |
| Pd105Di | Pd105Di | Pr141Di | CD45_141 |
| Pd106Di | Pd106Di | Nd142Di | CD19 |
| Pd108Di | Pd108Di | Nd144Di | CD141 |
| Pd110Di | Pd110Di | Nd145Di | CD4 |
| Nd144Di | CD141 | Nd146Di | CD8 |
| Nd148Di | CD16 | Nd148Di | CD16 |
| Nd150Di | pSTAT5 | Sm149Di | CD66 |
| Eu151Di | CD123 | Nd150Di | p-STAT5 |
| Sm152Di | CD66b | Eu151Di | CD123 |
| Eu153Di | CD1c | Eu153Di | CD1c |
| Tb159Di | CD45_159 | Sm154Di | CD163 |
| Gd160Di | CD14 | Tb159Di | CD11c |
| Dy162Di | CD64 | Gd160Di | CD14 |
| Ho165Di | CD45_165 | Dy162Di | CD32 |
| Tm169Di | CD45_169 | Ho165Di | CD116 |
| Er170Di | CD3 | Er166Di | CD24 |
| Yb172Di | CD11b | Er167Di | CD38 |
| Yb173Di | CD56 | Er168Di | CD206 |
| Yb174Di | HLA-DR | Tm169Di | CD131 |
| Lu175Di | CD45_175 | Er170Di | CD3 |
| Ir191Di | DNA | Yb173Di | CD56 |
| Ir193Di | DNA | Yb14Di | HLADR |
| | | Ir191Di | DNA |
| | | Ir193Di | DNA |

Sera were used at the same dilution, without GM-CSF titer adjustment, to reflect ex vivo conditions. Reduced STAT5 phosphorylation was observed in monocytes, DC and pDCs when adding GM-CSF in the presence of sera from PAP and anti-GM-CSF positive CD patients, compared to sera from anti-GM-CSF negative CD patients (FIG. 1D and FIG. 5E). No activation of STAT5 was recorded in TB/NK cells and only minor responses were seen in basophils upon GM-CSF stimulation in all experimental group (FIG. 5E). Of note, anti-GM-CSF autoantibodies had no effect on the stimulation of basophils with IL-3, suggesting no direct effect of these antibodies on CSF2RB-associated cytokine signaling (FIG. 5F). Reduced levels of STAT5 phosphorylation correlated with increased titers of anti-GM-CSF autoantibodies (FIG. 1E). These data collectively demonstrate the presence of neutralizing antibodies in CD patients affecting GM-CSFR signaling in blood-derived myeloid cells, including circulating precursors of intestinal antigen-presenting cells (APC).

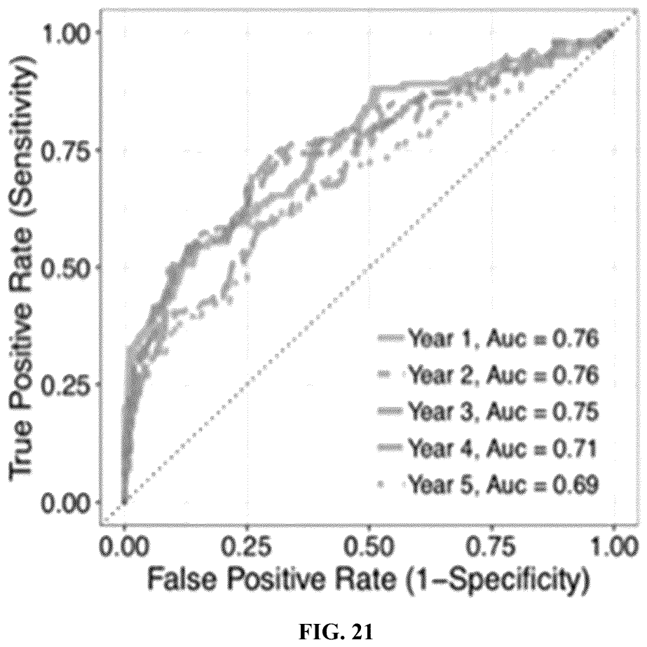

FIG. 21 shows the predictive performance of anti-flagellin X and ASCA-IgA antibody markers in terms of receiver operator curves (ROC) for years 1, 2, 3, 4, and 5 before diagnosis.

### Example 4—CD-Associated Anti-GM-CSF Antibodies Precede the Onset of Severe Disease

The presence of neutralizing anti-GM-CSF IgA and IgG2 antibodies in CD patients suggests it may contribute to not only disease mechanism but possibly etiology as well. In order to address this hypothesis, sera obtained from the Department of Defense Serum Repository were tested, prospectively collected from military service members during their annual routine medical examinations. Some of these service members were eventually diagnosed with either UC or CD during the course of their service. Three to four longitudinal serum samples spanning up to 10 years, obtained prior and up to and post diagnosis from 220 CD, 200 UC, and 200 matched individuals remaining healthy (HD), were analyzed in two independent runs (Table 3).

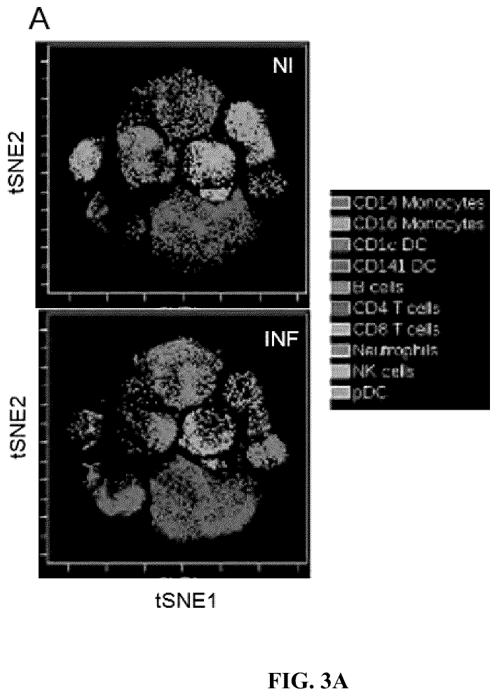

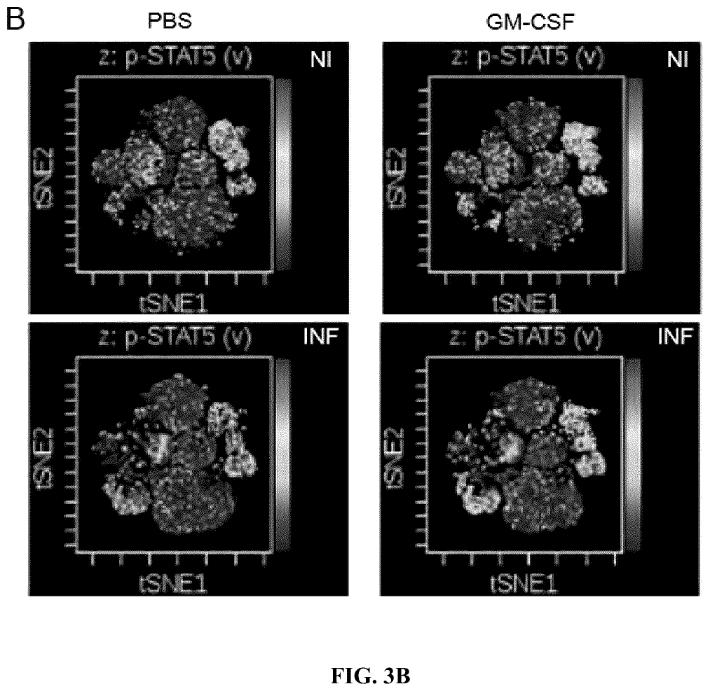

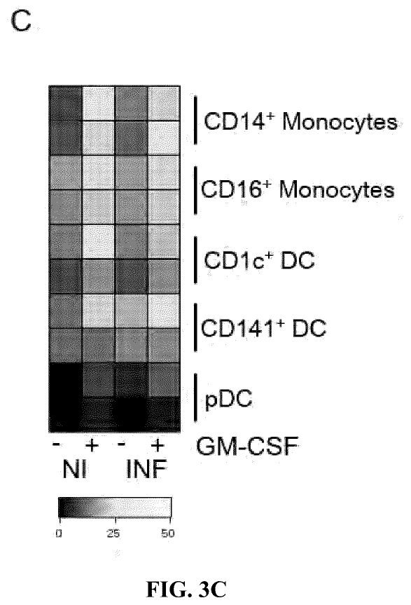

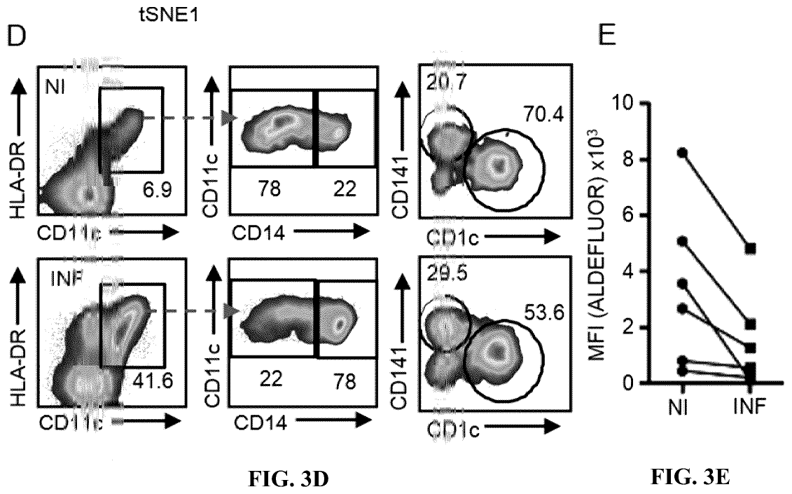

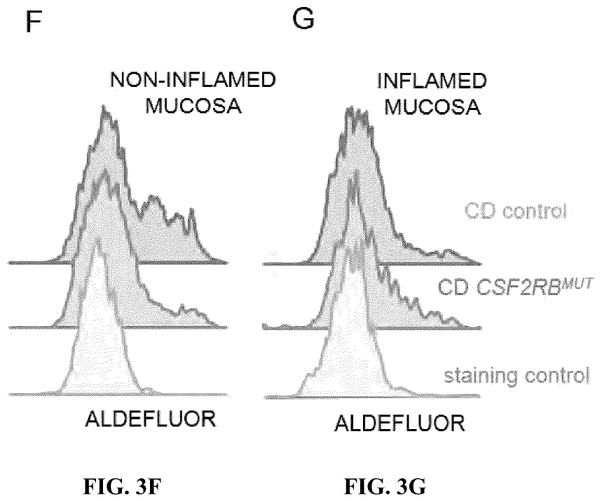

Table 3 shows available patient information for serum samples used in FIGS. 3A-3G, FIGS. 7A-7F and FIGS. 8A-8F.

TABLE 3
Patient Information for Serum Samples Used in
FIGS. 3A-3G, FIGS. 7A-7F and FIGS. 8A-8F.

| | Training cohort | | | Validation cohort | | |
|---|---|---|---|---|---|---|
| | CD | UC | HC | CD | UC | HC |
| N subjects | 120 | 100 | 100 | 100 | 100 | 100 |
| N samples | 360 | 300 | 300 | 400 | 400 | 400 |
| Mean age | 30.8 | 29.11 | 29.18 | 31.9 | 32.2 | 34.39 |
| Sex (M) | 79% | 100% | 100% | 78% | 80% | 97% |
| % complication | 28.3% | — | — | 28% | — | — |
| L1 | 27.5 | | | 11% | — | — |
| L2 | 15.8 | | | 33% | — | — |
| L3 | 39.1 | | | 30% | — | — |
| Unknown | 17.5 | | | 26% | — | — |
| location | | | | | | |

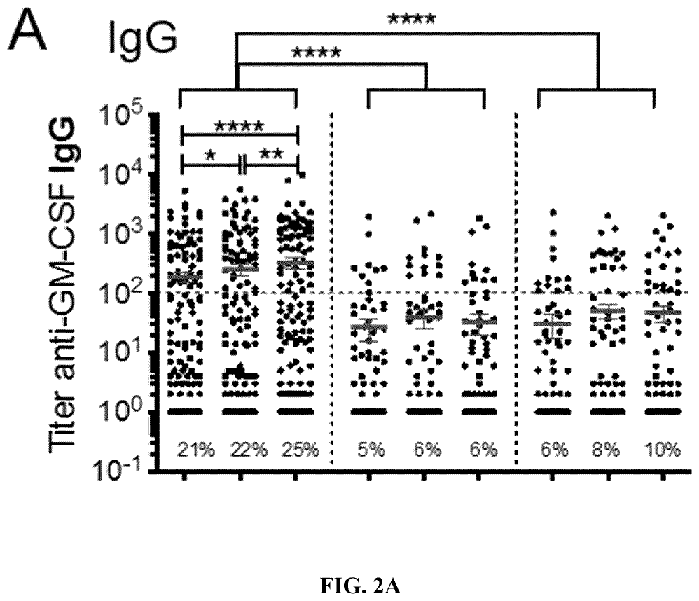

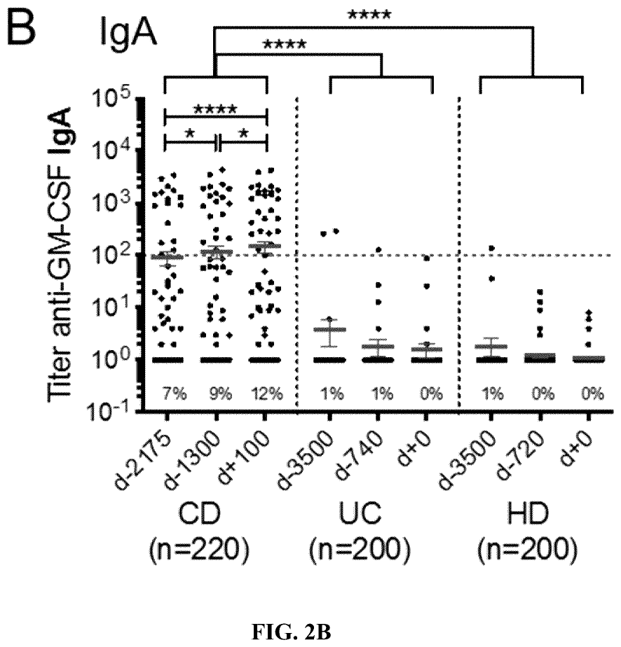

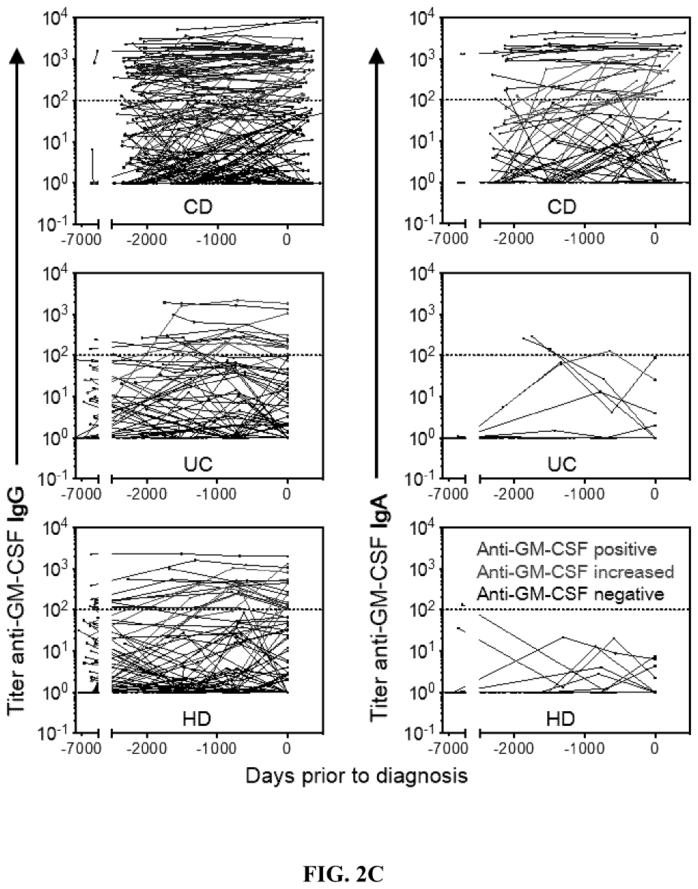

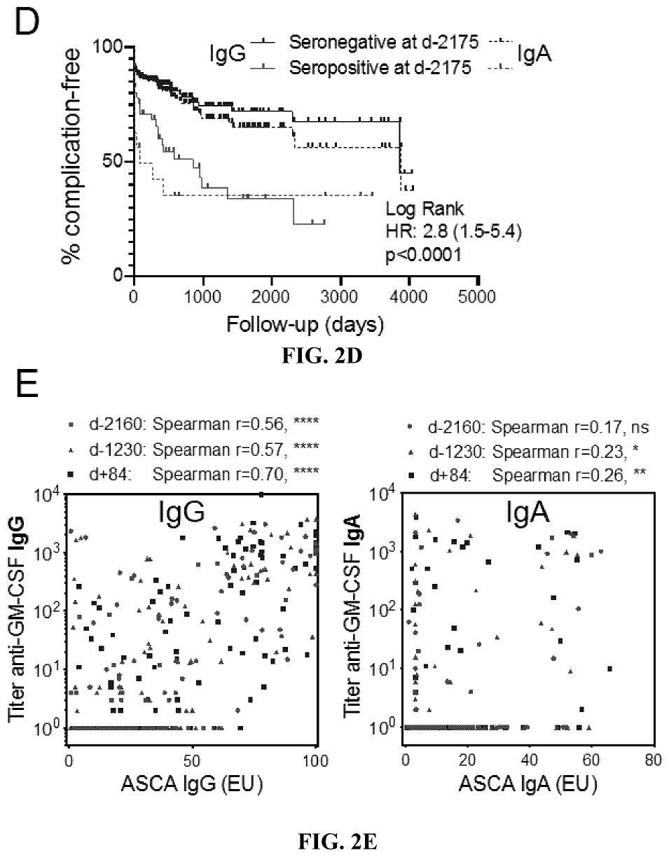

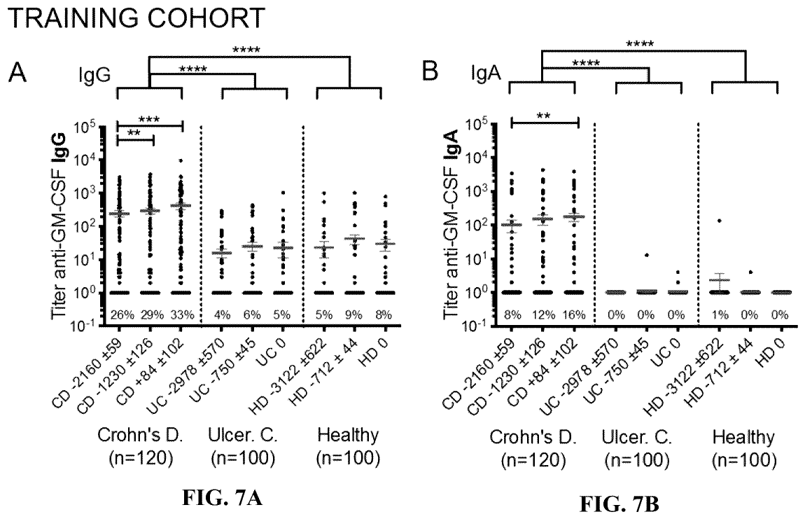

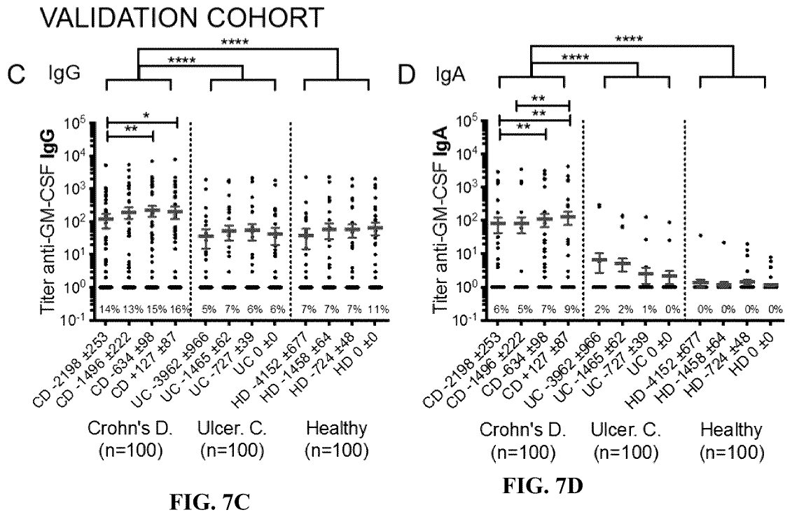

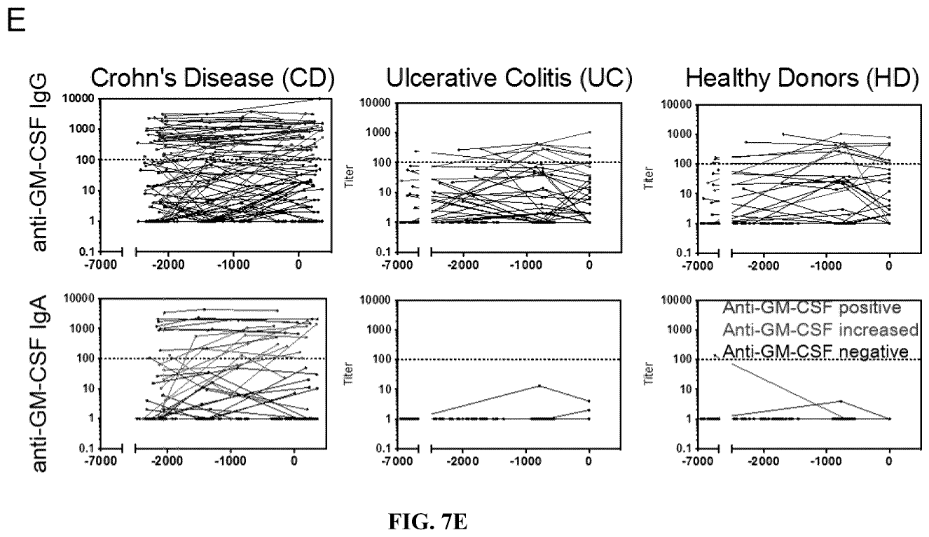

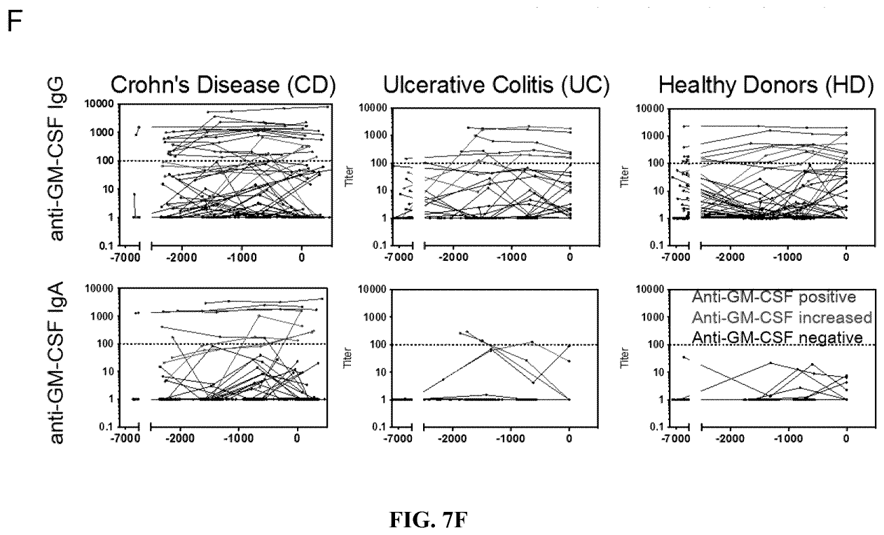

Isotype specific anti-GM-CSF ELISAs (total IgA and IgG) were performed for each time point of collection. Healthy military service members and service members eventually diagnosed with UC had a similar 5-10% detection rate of anti-GM-CSF IgG, with low mean titers below the limit of significance (between 1/25 and 1/50), and nearly no anti-GM-CSF IgA detection (0-1%), without significant change by time point (FIG. 2A-2B and FIG. 7A-7D). In contrast and remarkably, IgG and IgA to GM-CSF were already found 6 years prior to CD diagnosis in 21% and 7% of samples, with additional patients seroconverting and with mean titers significantly increasing from 1/190 to 1/320 as the date of diagnosis approaches (FIG. 2A-2C and FIG. 7A-7F). At time of diagnosis, anti-GM-CSF IgA autoantibodies were exclusively elevated in 12% of CD, while IgG were significantly more frequent (25%) in CD compared to HD and UC (FIG. 2A-2B and FIG. 7C). Nearly all CD patients with detectable anti-GM-CSF autoantibodies 6 years prior to diagnosis maintained or increased their titers over time as symptoms of CD approached (FIG. 2C and FIGS. 7E-7F). Most (75%) anti-GM-CSF IgA co-occurred with IgG, while IgG to GM-CSF was more frequent and detected in 63% of cases in the absence of IgA.

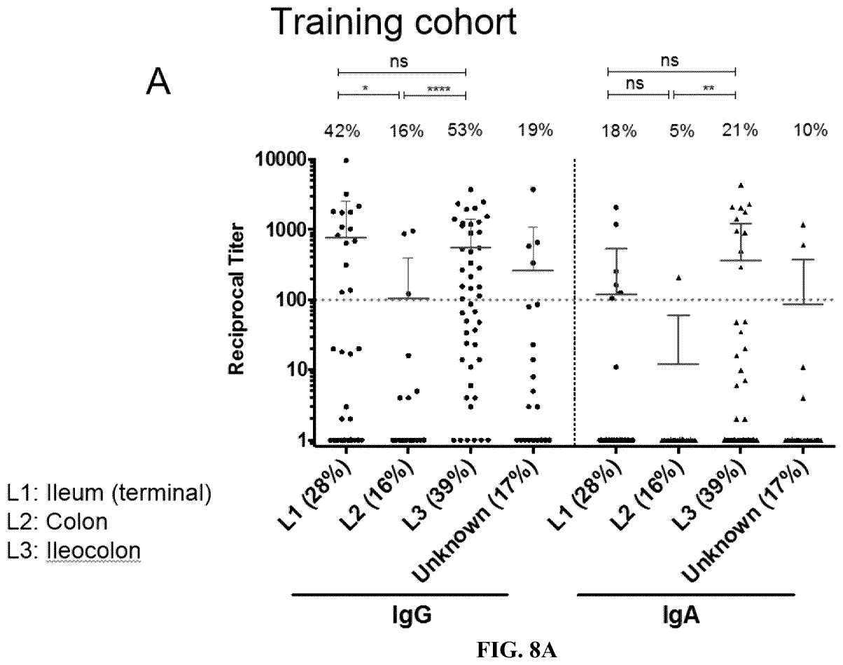

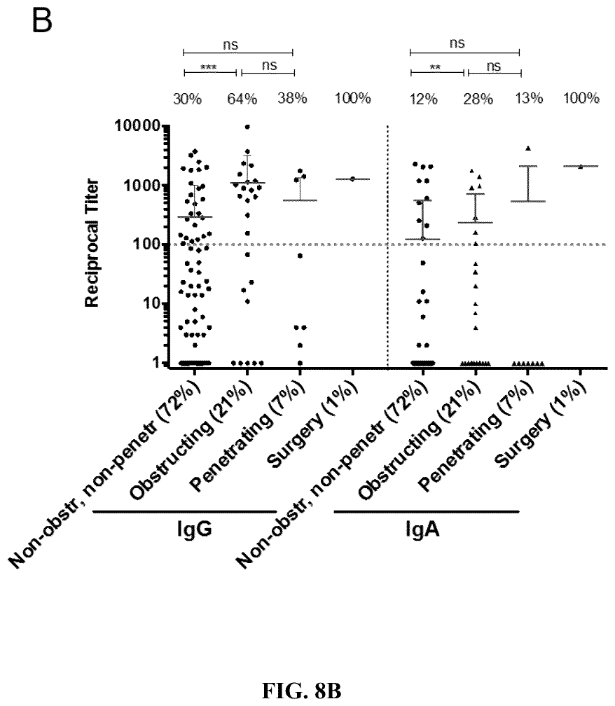

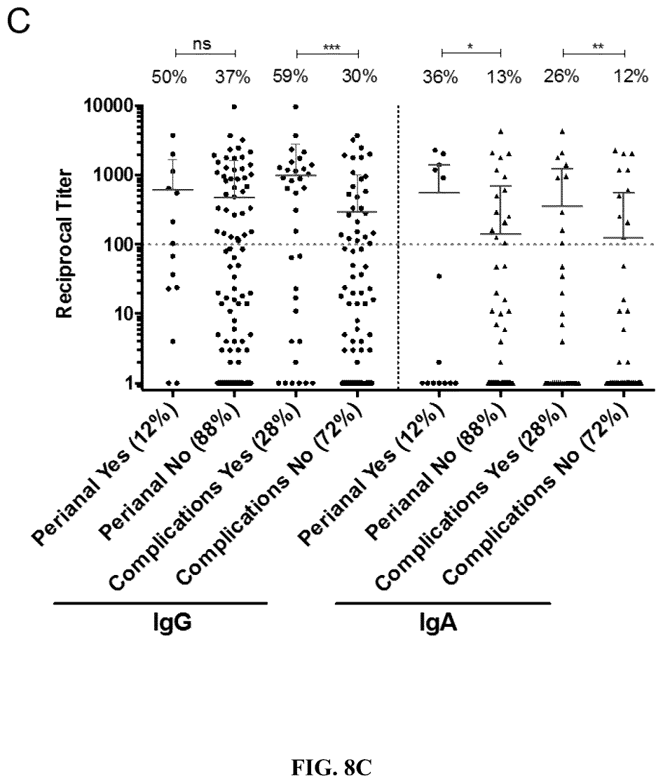

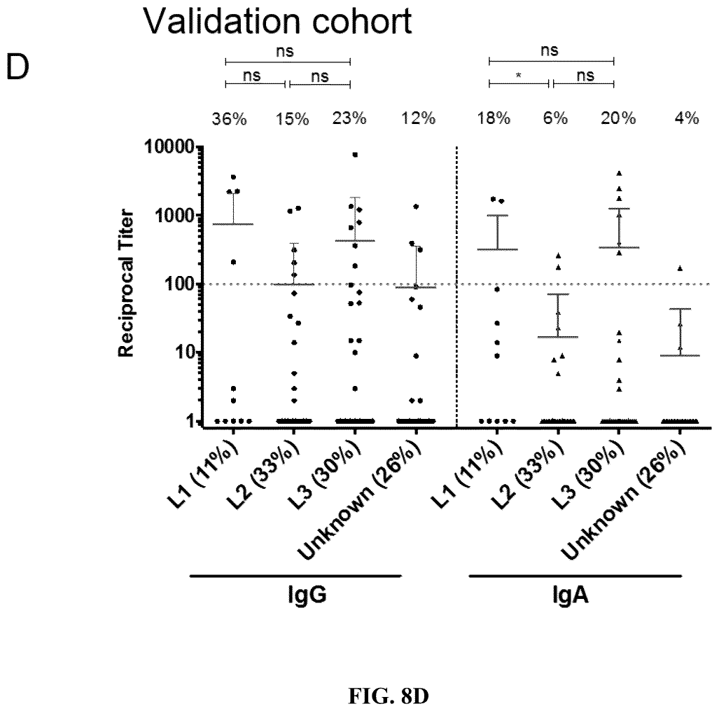

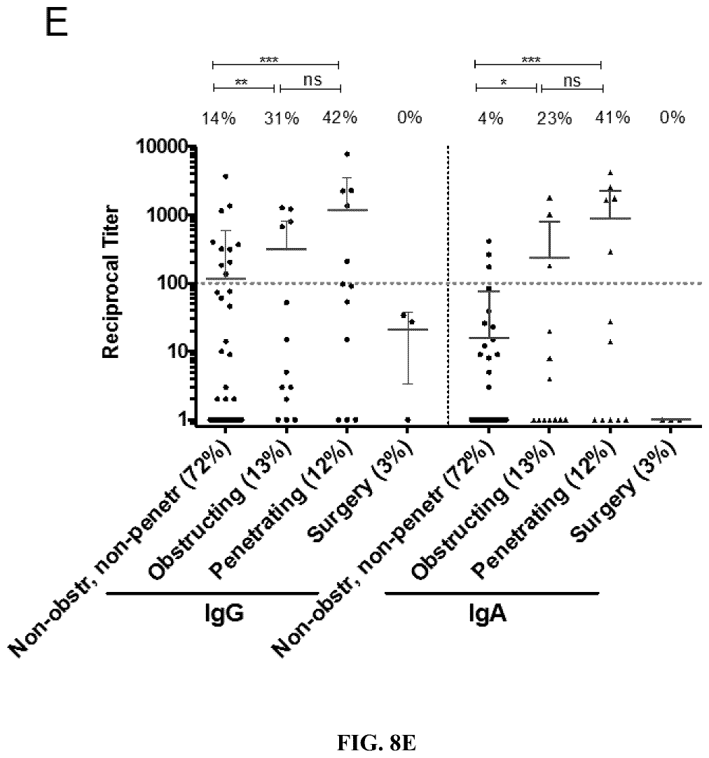

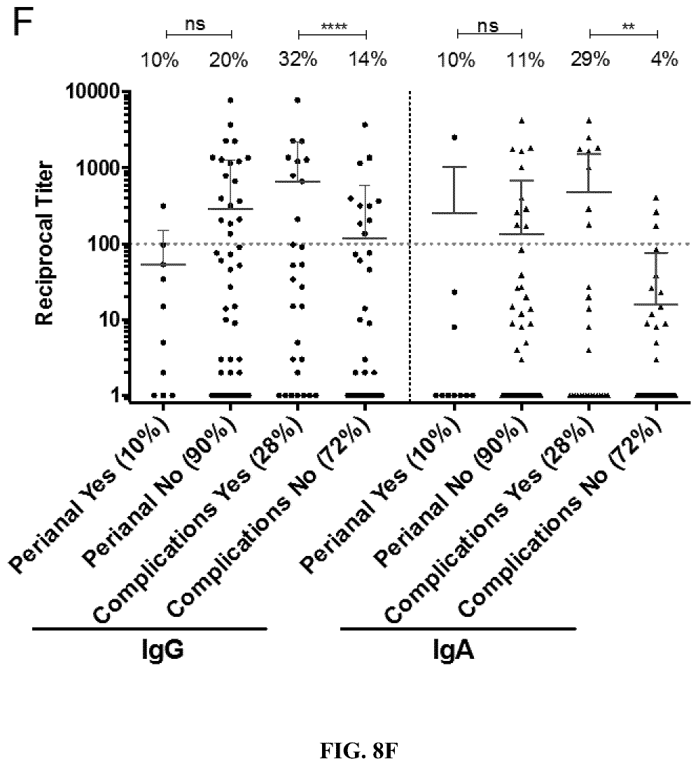

Most importantly, presence of IgG or IgA in CD was associated with ileal/ileocolonic involvement and with more severe disease and complications within 100 days of diagnosis, with a 2.8 risk hazard ratio of having constricting and/or structuring disease or require surgery soon after symptoms appear (FIG. 2D and FIGS. 8A-8D). These findings demonstrate that CD-associated anti-GM-CSF autoantibodies are not only detectable years before the onset of full disease manifestation, but also correlate with severity of disease at onset, making anti-GM-CSF autoantibodies a possible predictive biomarker for the development of complicated CD in a subset of patients. Indeed, almost all patients with anti-GM-CSF IgA had complicated disease occurring within the first 100 days of diagnosis (FIG. 2D). Presence of IgA up to 6 years prior to diagnosis provided a predictor for CD development with >97% specificity and with sensitivity increasing from 15% to 21% as diagnosis nears (ROC 0.6). Interestingly, the detection of anti-GM-CSF autoantibodies did not correlate with date of birth, sex, race, or year of sample acquisition, rendering this biomarker ubiquitously applicable across patients. Anti-Saccharomyces cerevisiae antibodies (ASCA) are a commonly used serological marker for IBD (Plevy et al., “Combined Serological, Genetic, and Inflammatory Markers Differentiate non-IBD, Crohn's Disease, and Ulcerative Colitis Patients,” Inflammatory Bowel Disease 19:1139-1148 (2013) and Silverberg et al., “Toward an Integrated Clinical, Molecular and Serological Classification of Inflammatory Bowel Disease: Report of a Working Party of the 2005 Montreal World Congress of Gastroenterology,” Canadian Journal of Gastroenterology 19 Suppl A:5A-36A (2005), both of which are hereby incorporated by reference in their entirety), and it was recently also found they are present prior to diagnosis using similar cohorts (Tones et al., “Serum Biomarkers Identify Patients Who Will Develop Inflammatory Bowel Diseases Up to 5 Y Before Diagnosis,” Gastroenterology 5085:30327-30329 (2020), which is hereby incorporated by reference in its entirety). Next, it was tested whether anti-GM-CSF autoantibodies in CD patients correlate with ASCA (IgA and IgG) at 2000 days, 500 days prior to diagnosis and 100 days post diagnosis (FIG. 2E and Table 4). Strikingly, anti-GM-CSF autoantibodies preceded the occurrence of anti-ASCA IgA antibodies and showed no significant correlations at −2000 days prior to diagnosis (FIG. 2E and Table 4).

Table 4 shows that ELISAs for ASCA-specific antibodies were performed on serum samples collected at three different time points (time point 1 and 2=prior to disease diagnosis, time point 3 post disease diagnosis). ASCA-specific IgG and IgA were measured.

TABLE 4
ELISAs for ASCA-specific Antibodies Performed on Serum Samples

| | Healthy Controls | Ulcerative Colitis | Crohn's Disease |
|---|---|---|---|
| ASCA-IgG | Spearman r = 0.40; | Spearman r = 0.35; | Spearman r = 0.56; |
| (−2000 | p < 0.0001 **** | p = 0.0003 *** | p < 0.0001 **** |
| days) | | | |
| ASCA-IgG | Spearman r = 0.38; | Spearman r = 0.31; | Spearman r = 0.57; |
| (−500 days) | p < 0.0001 **** | p = 0.0019 ** | p < 0.0001 **** |
| ASCA-IgG | Spearman r = 0.48; | Spearman r = 0.29; | Spearman r = 0.70; |
| (+100 days) | p < 0.0001 **** | p = 0.0034 ** | p < 0.0001 **** |
| ASCA-IgA | Spearman r = 0.18; | Spearman r = 0.18; | Spearman r = 0.17; |
| (−2000 | n.s. | n.s. | n.s. |
| days) | | | |
| ASCA-IgA | Spearman r = 0.15; | Spearman r = 0.18; | Spearman r = 0.23; |
| (−500 days) | n.s. | n.s. | p = 0.0115 * |
| ASCA-IgA | Spearman r = N/A; | Spearman r = 0.17; | Spearman r = 0.26; |
| (+100 | n.s. | n.s. | p = 0.0049 ** |
| days) | | | |

These data suggest serum anti-GM-CSF antibodies, particularly anti-GM-CSF IgA, to be a potential predictor of CD, disease location and risk of disease complications in a larger group of CD patients.

### Example 5—The CD Mucosa Shows Impaired Homeostatic Functions in GM-CSF-Responsive Myeloid Cells

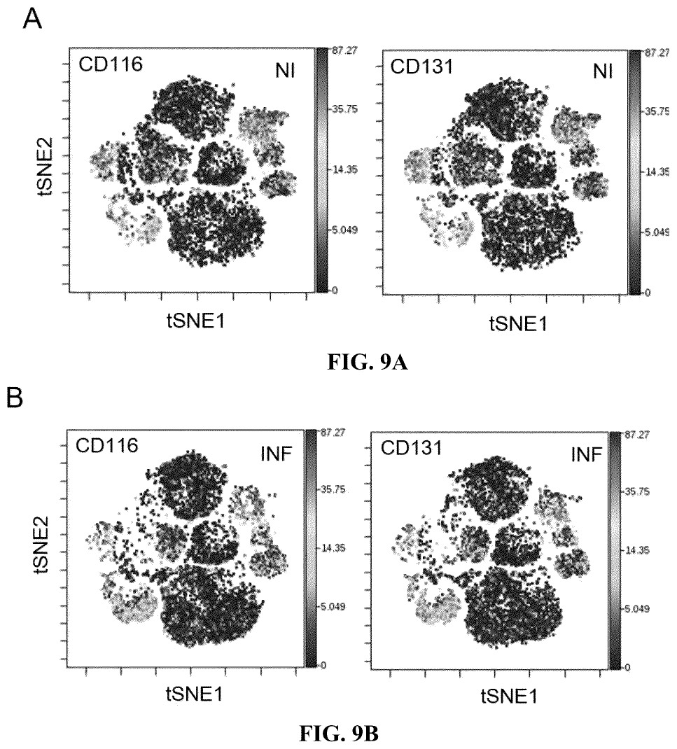

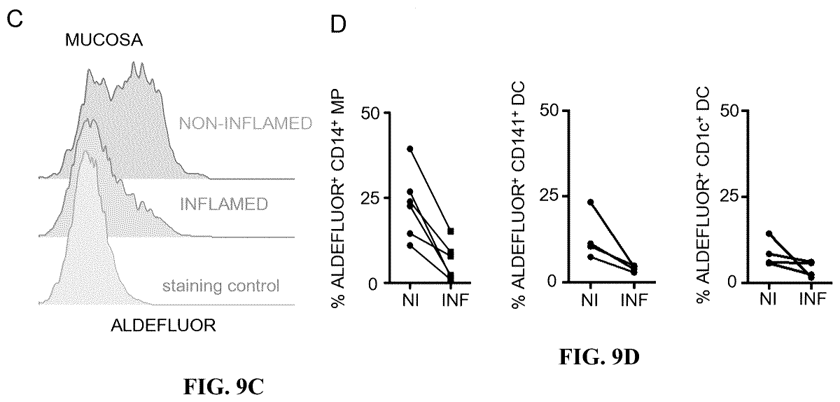

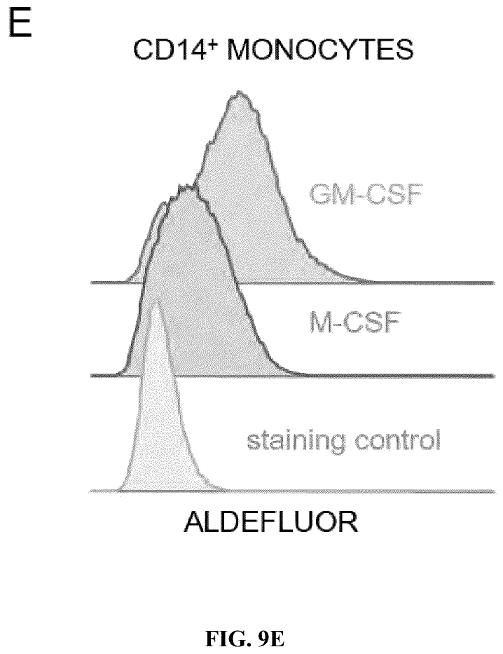

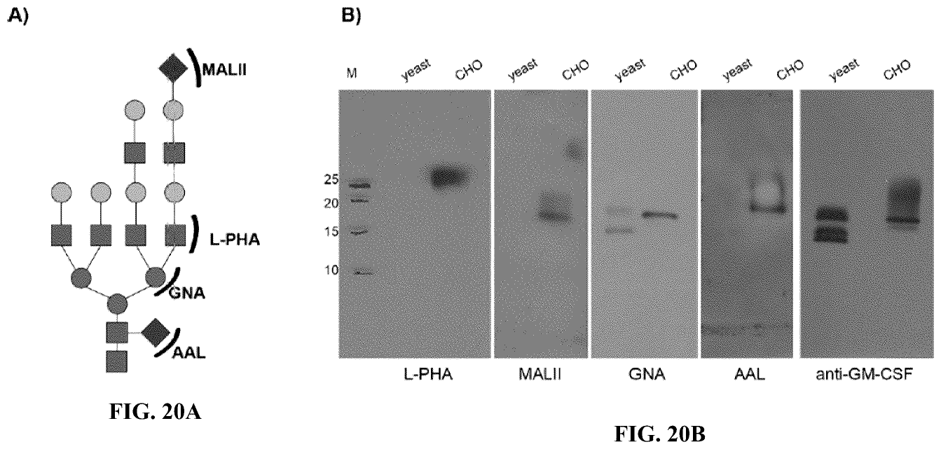

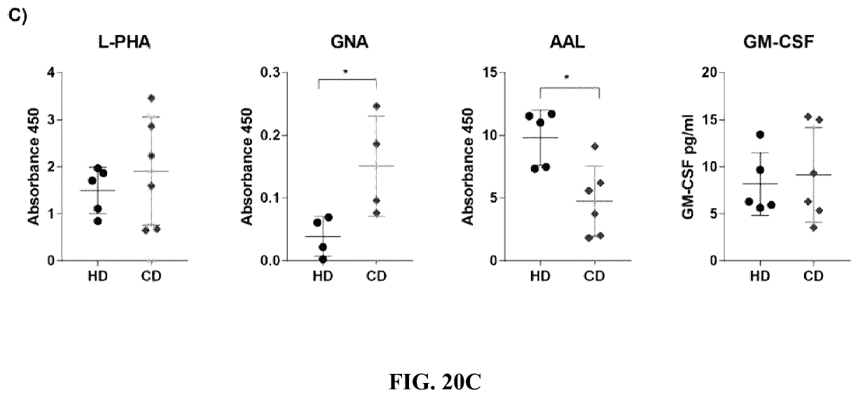

GM-CSF engages the heterodimeric GM-CSF receptor (GM-CSFR), composed of the GM-CSF binding alpha chain CD116 (CSF2RA) and the signal transducing common beta chain CD131 (CSF2RB) to induce downstream activation of the transcription factor STATS. To understand mechanisms of GM-CSF impairment, a 28-parameter mass cytometry panel was used and the distribution of CD116 and CD131 on hematopoietic cells across the inflamed (INF) and non-inflamed (NI) CD mucosa was determined (FIG. 20). Analysis highlighted differences in the abundance of CD16⁺ monocytes, CD14⁺ monocytes, CD141⁺ DC, CD1⁺DC, plasmacytoid DC (pDC) and neutrophils between IFN and NI CD mucosa (FIG. 3A), but failed to determine inflammation-dependent differences in CD116 and CD131 expression (FIGS. 9A and 9B). In line with an unperturbed GM-CSFR expression, assessment of GM-CSF responsiveness in freshly isolated lamina propria leukocytes obtained from the NI and INF CD mucosa revealed potent STATS phosphorylation across all GM-CSFR-expressing cells following stimulation with Sargramostim, independent of the inflammation in the tissue (FIG. 3B). Collectively, the functional characterization of the GM-CSFR on myeloid cells isolated from NI and INF CD tissues revealed unperturbed GM-CSFR expression and responsiveness (FIG. 3B and 3C).

GM-CSF stimulation controls steady state functions of intestinal macrophages and DC. Next, the production of retinoic-acid (RA), an essential homeostatic function of antigen-presenting cells (APCs) to sustain intestinal immune balance and induce the expression of gut-homing receptors in lymphocytes was validated. Hall et al., “The Role of Retinoic Acid in Tolerance and Immunity,” Immunity 35:13-22 (2011), which is hereby incorporated by reference in its entirety. ALDEFLUOR staining on HLA-DR⁺CD11⁺ APCs obtained from the INF and NI CD mucosa revealed a substantial decrease in RA production by APCs specifically in the INF mucosa (FIGS. 3D, 3E and FIG. 9C). Decreased levels of ALDEFLUOR staining were observed in CD14⁺ MP, CD141⁺ DC and CD1⁺DC (FIG. 9D). Monocytes and precursor DC continuously infiltrate the intestinal mucosa to differentiate into DC and MP depending on the locally available cytokine milieu. Bujko et al., “Transcriptional and Functional Profiling Defines Human Small Intestinal Macrophage Subsets,” The Journal of Experimental Medicine 215:441-458 (2018) and Richter et al., “Transcriptional Profiling Reveals Monocyte-Related Macrophages Phenotypically Resembling DC in Human Intestine,” Mucosal Immunology 11:1512-1523 (2018), which are hereby incorporated by reference in their entirety. It was demonstrated that myeloid cells in GM-CSF deficient mice or isolated from CD patients carrying a frame-shift mutation in CSF2RB, display impaired RA production. Mortha et al., “Microbiota-dependent Crosstalk Between Macrophages and ILC3 Promotes Intestinal Homeostasis,” Science 343:1249288 (2014) and Chuang et al., “A Frameshift in CSF2RB Predominant Among Ashkenazi Jews Increases Risk for Crohn's Disease and Reduces Monocyte Signaling via GM-CSF,” Gastroenterology 151:710-723 (2016), both of which are hereby incorporated by reference in their entirety. In line with these findings, culturing of CD14⁺ blood-derived monocytes in the presence of GM-CSF confirmed a GM-CSF-dependent increase in ALDEFLUOR staining in monocyte-derived myeloid cells (FIG. 9E). These levels were comparable to levels observed in APCs obtained from the NI CD mucosa (FIG. 9C). To confirm the requirement of GM-CSFR signaling for RA production by APCs in situ, APCs were isolated from a biopsy collected from one patient carrying a frame-shift mutation in CSF2RB. APCs in both the NI and INF mucosa of this patient showed low levels of RA production compared to APCs isolated from a biopsy of a patient with an intact CSF2RB gene, suggesting a critical role for the GM-CSF-GM-CSFR axis in sustaining homeostatic APC functions (FIGS. 3F and 3G). Collectively, these data demonstrate an essential role for GM-CSF in controlling the homeostatic production of RA by gut APC.

### Example 6—T Cells and ILC3 Contribute to the Pool of GM-CSF in the Non-Inflamed and Inflamed CD Mucosa

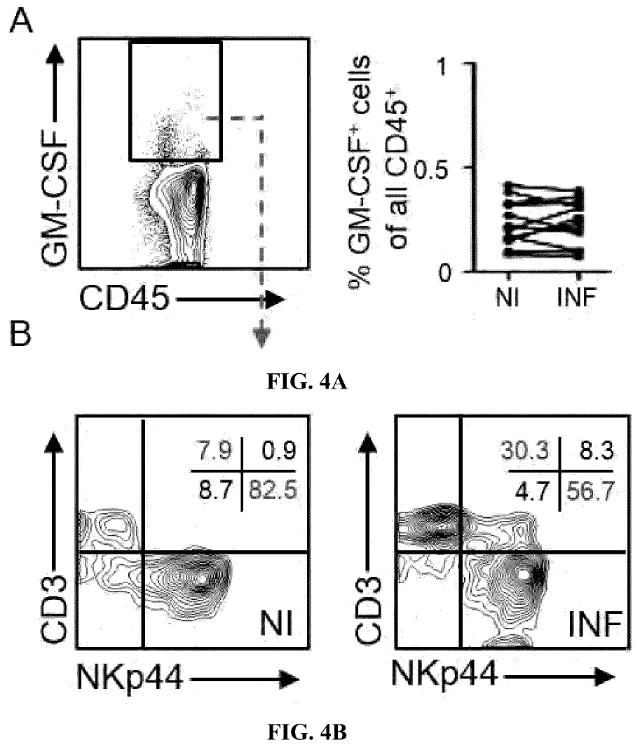

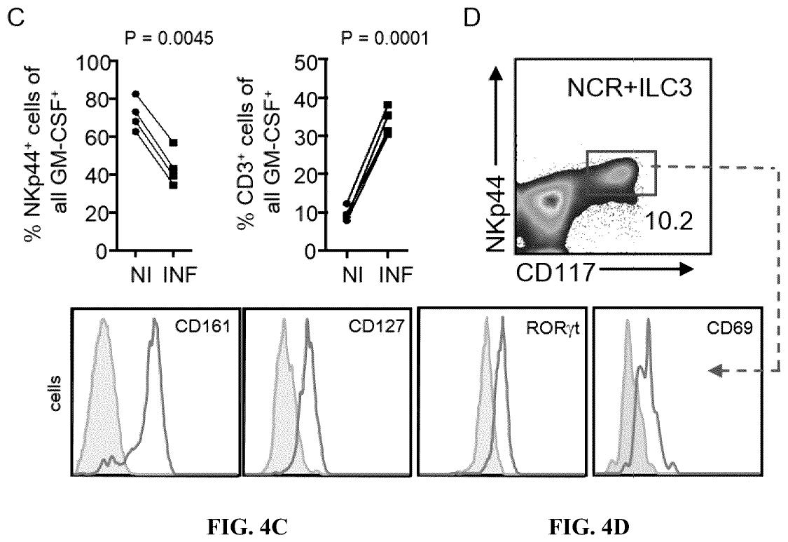

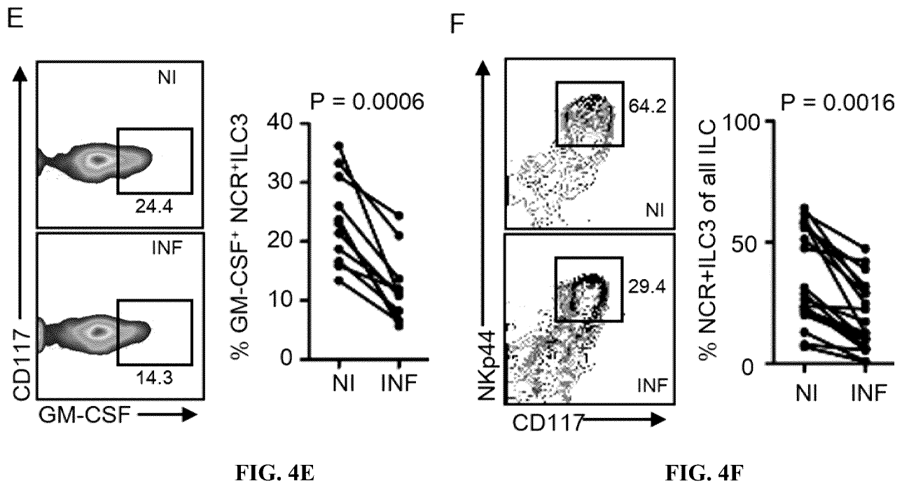

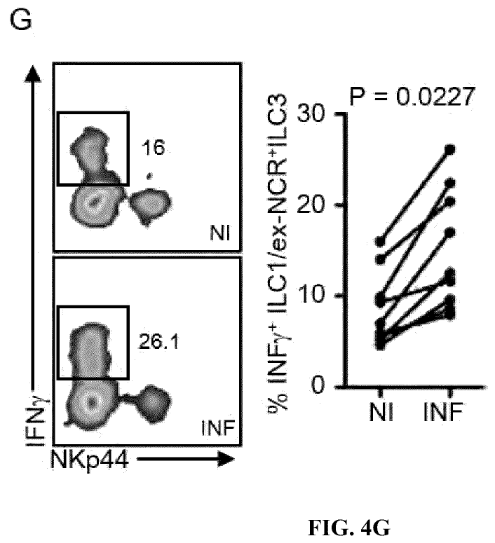

Next, the source of GM-CSF in NI and INF ileal resections obtained from CD patients (n=12) was examined. Intracellular cytokine staining in freshly isolated lamina propria leukocytes revealed no significant changes in the overall spontaneously release of GM-CSF by CD45⁺cells (FIG. 4A). A closer characterization of GM-CSF-producing cells in the NI CD mucosa revealed NKp44 expression on 80% of these cells, while the remaining 20% either expressed CD3 (approx. 10%), or remained negative for both markers (approx. 10%) (FIG. 4B). Importantly, the composition of GM-CSF-producing cells in the ileal INF CD mucosa revealed an increase in spontaneously released GM-CSF by CD3⁺ T cells at the expense of GM-CSF-producing NKp44⁺ CD3⁻(FIGS. 4B and 4C). GM-CSF-secreting NKp44⁺ CD3⁻ cells co-expressed CD117, CD127, CD161, CD69 and the transcription factor Retinoic acid-related Orphan Receptor (ROR) gamma (γ) t (FIG. 4D), identifying them as natural cytotoxicity receptor (NCR)⁺group 3 innate lymphoid cells (ILC3) (NCRALC3). Cupedo et al., “Human Fetal Lymphoid Tissue-Inducer Cells are Interleukin 17-producing Precursors to RORC+CD127+Natural Killer-Like Cells,” Nature Immunology 10:66-74 (2009); Cella et al., “A Human Natural Killer Cell Subset Provides an Innate Source of IL-22 for Mucosal Immunity,” Nature 457:722-725 (2009); and Glatzer et al., “RORgammat(+) Innate Lymphoid Cells Acquire a Proinflammatory Program Upon Engagement of the Activating Receptor NKp44,” Immunity 38:1223-1235 (2013), all of which are hereby incorporated by reference in their entirety. These findings are in line with previously reported sources of GM-CSF in the human and murine intestinal lamina propria. Mortha et al., “Microbiota-dependent Crosstalk Between Macrophages and ILC3 Promotes Intestinal Homeostasis,” Science 343:1249288 (2014) and Cella et al., “A Human Natural Killer Cell Subset Provides an Innate Source of IL-22 for Mucosal Immunity,” Nature 457:722-725 (2009), both of which are hereby incorporated by reference in its entirety. Next, it was wondered whether the decrease in GM-CSF +NCR⁺ILC3 cells was due to a decrease in GM-CSF production, or a decrease in the abundance of NCR⁺ILC3 within the INF mucosa. The interrogations, analyzing NCR⁺ILC3 numbers and the per cell release of GM-CSF by flow cytometry, revealed a decrease in GM-CSF production by NCR⁺ILC3 and a lower abundance of NCR⁺ILC3s in the INF mucosa (FIGS. 4E and 4F). Downregulation of RORγt and differentiation of NCR⁺ILC3 into inflammatory group 1 ILCs (ILC1) or ex-RORγt NCR⁺ILC3 has previously been reported. Vonarbourg et al., “Regulated Expression of Nuclear Receptor RORgammat Confers Distinct Functional Fates to NK Cell Receptor-Expressing RORgammat(+) Innate Lymphocytes,” Immunity 33:736-751 (2010) and Bernink et al., “Human type 1 Innate Lymphoid Cells Accumulate in Inflamed Mucosal Tissues,” Nature Immunology 14:221-229 (2013), both of which are hereby incorporated by reference in their entirety. Interestingly, this analysis of ex-RORγt NCR⁺ILC3/ILC1 cells obtained from the NI and IFN CD mucosa reveal higher levels of the IBD-associated inflammatory cytokine interferon (IFN)γ in the INF mucosa (FIG. 4G). Abraham and Cho, “Inflammatory Bowel Disease,” The New England Journal of Medicine 361:2066-2078 (2009) and Glatzer et al., “RORgammat(+) Innate Lymphoid Cells Acquire a Proinflammatory Program Upon Engagement of the Activating Receptor NKp44,” Immunity 38:1223-1235 (2013), both of which are hereby incorporated by reference in their entirety. Noteworthy, IFNγ was virtually absent in NCR⁺ILC3 compared to ex-RORγt NCR⁺ILC3/ILC1 (FIG. 4G). Collectively, these findings demonstrate a change in the source and per cell output of GM-CSF in the INF CD mucosa, accompanied by a switch from homeostatic NCR⁺ILC3-derived GM-CSF, towards ex-RORγt NCR⁺ILC3/ILC1-associated IFNy (FIG. 4E-4G). It is reported that ILC3-derived GM-CSF contributes to the homeostatic production of RA by myeloid cells in mice. Mortha et al., “Microbiota-dependent Crosstalk Between Macrophages and ILC3 Promotes Intestinal Homeostasis,” Science 343:1249288 (2014) and Samarakoon et al., “CD45 Regulates GM-CSF, Retinoic Acid and T-cell Homing in Intestinal Inflammation,” Mucosal Immunology 9:1514-1527 (2016), both of which are hereby incorporated by reference in their entirety. The reduced availability of NCR⁺ILC3-derived GM-CSF in the INF mucosa of CD patients may account for the decrease production of RA observed in APCs isolated from these tissues (FIG. 3E-3G and FIG. 9C-9D). Interestingly, myeloid cell-derived RA has previously been shown to abrogate the downregulation of RORγt in human NCR⁺ILC3 and prevents the accumulation of inflammatory ex-RORγt NCR⁺ILC3/ILC1 in the inflamed CD mucosa. Bernink et al., “Interleukin-12 and -23 Control Plasticity of CD127(+) Group 1 and Group 3 Innate Lymphoid Cells in the Intestinal Lamina Propria,” Immunity 43:146-160 (2015) and Bernink et al., “Human type 1 Innate Lymphoid Cells Accumulate in Inflamed Mucosal Tissues,” Nature Immunology 14:221-229 (2013), both of which are hereby incorporated by reference in their entirety. Considering the dependency of myeloid RA-production on GM-CSF, anti-GM-CSF autoantibodies or defective GM-CSFR signaling in myeloid cells may therefore change the abundance of tissue-resident ILC subsets and confer a transition from homeostatic to pre-diseased tissue state. Collectively, these findings suggest an altered balance within the sources of GM-CSF and its impact on the myeloid cell homeostasis. Anti-GM-CSF autoantibodies with a predominant mucosal isotype profile, specific to posttranslational modifications are a serological marker occurring prior to the onset of CD and may alter important this tissue-resident immune balance long before a clinical manifestation of the disease is established. These observations open the door to potential treatments delaying or even preventing the onset of CD in patients with anti-GM-CSF autoantibodies.

### Example 7—Unmodified GM-CSF as a Potential Way to Restore Homeostatic Functions of GM-CSF

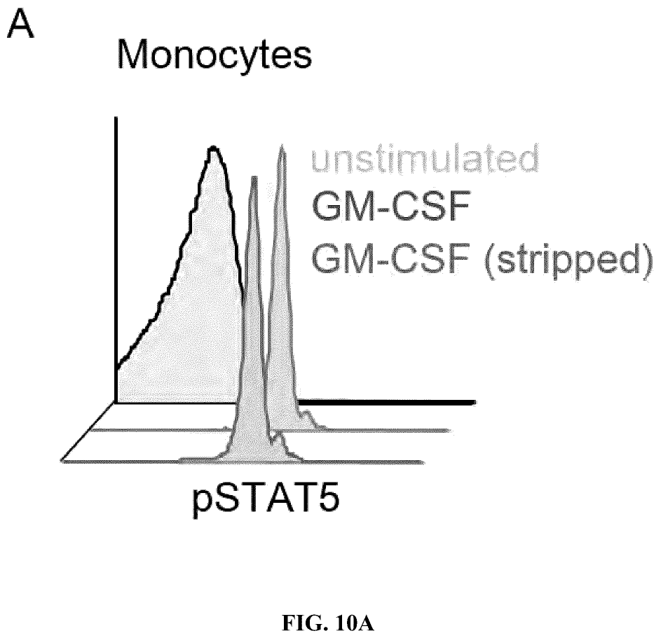

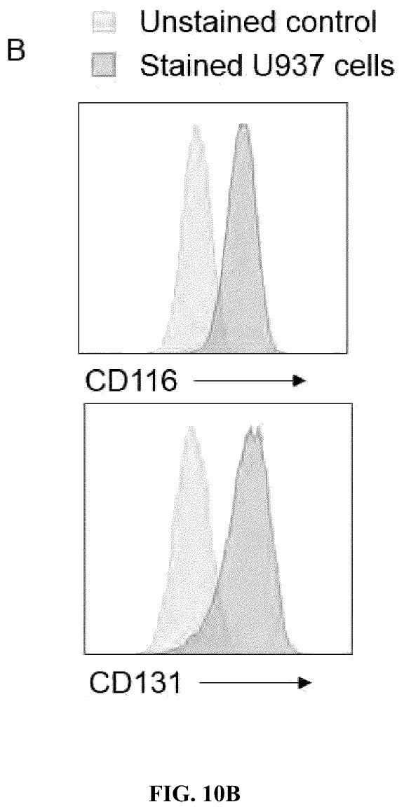

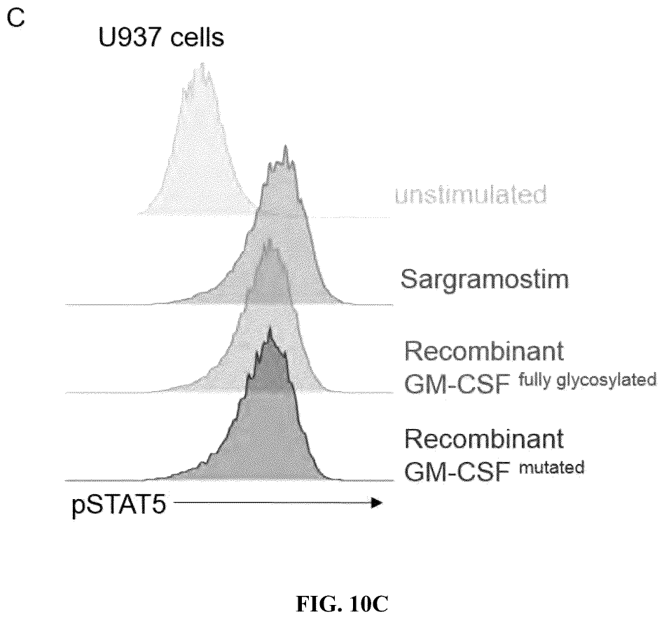

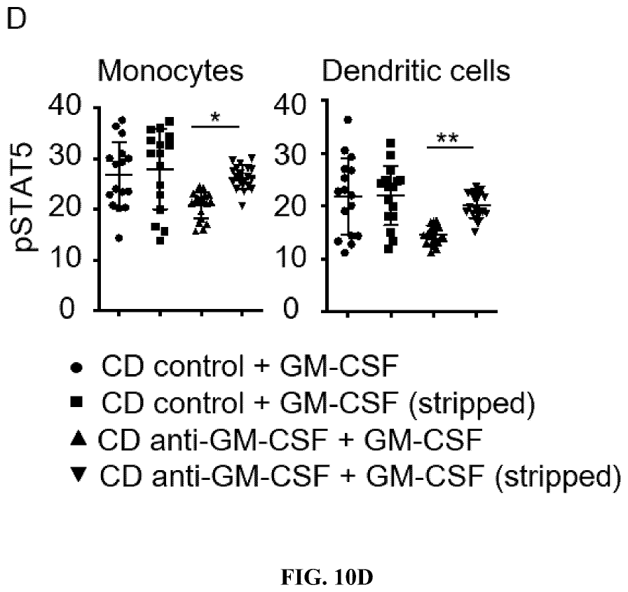

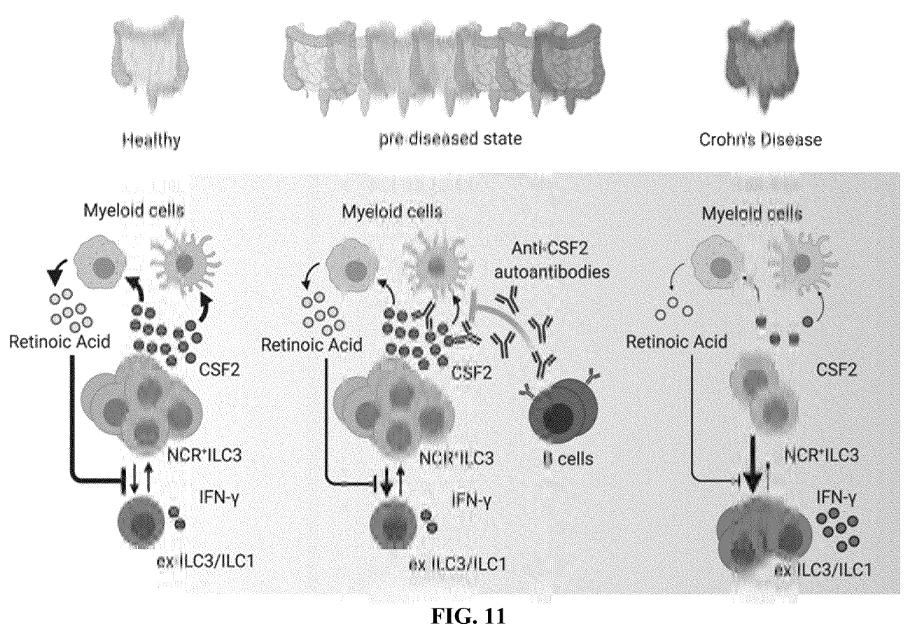

Enzymatically treated GM-CSF, or genetically engineered GM-CSF lacking all posttranslational glycosylations, retain their biological activity and initiate GM-CSFR-mediated phosphorylation of STATS in blood purified CD14⁺ monocytes or CD116/CD131-expressing monocytic U937 cells (FIGS. 10A-10C). It is hypothesized that stripping posttranslational modifications on GM-CSF would render the cytokine capable of escaping neutralization by CD-associated anti-GM-CSF autoantibodies while retaining its biological activity on myeloid cells. To test this, freshly isolated PBMCs were stimulated with GM-CSF or enzymatically stripped GM-CSF in the presence or absence of neutralizing anti-GM-CSF autoantibodies from CD patients. Samples were analyzed using mass cytometry and STAT5 phosphorylation quantified in monocytes and DC (Table 2B). As shown before, untreated and enzymatically stripped GM-CSF retained their ability to lead to potent STAT5 phosphorylation even in the presence of CD serum lacking neutralizing anti-GM-CSF autoantibodies (FIGS. 10A-10D). The neutralizing effects of CD-associated anti-GM-CSF autoantibodies were recapitulated and resulted in a significant decrease in pSTAT5 signal in monocytes and DC upon stimulation with glycosylated GM-CSF (FIG. 10D). Strikingly, this decrease was reverted when monocytes and DC received cytokine stimulation through enzymatically stripped GM-CSF in the presence of neutralizing anti-GM-CSF autoantibodies (FIG. 10D). These findings suggest that functional forms of unmodified recombinant GM-CSF may be useful to restore myeloid cell homeostasis in CD patients developing spontaneous neutralizing anti-GM-CSF autoantibodies, offering a possible therapeutic perspective.

### Example 8—Discussion of Examples 1-7

GM-CSF is a critical factor controlling intestinal myeloid cell development and functions that sustain tissue immune homeostasis. Mortha et al., “Microbiota-dependent Crosstalk Between Macrophages and ILC3 Promotes Intestinal Homeostasis,” Science 343:1249288 (2014), which is hereby incorporated by reference in its entirety. Deficiencies in GM-CSFR signaling increase the susceptibility to infections and affect the outcome of diseases, suggesting an important role of GM-CSF in maintaining gut immune balance. Goldstein et al., “Defective Leukocyte GM-CSF Receptor (CD116) Expression and Function in Inflammatory Bowel Disease,” Gastroenterology 141:208-216 (2011) and Chuang et al., “A Frameshift in CSF2RB Predominant Among Ashkenazi Jews Increases Risk for Crohn's Disease and Reduces Monocyte Signaling via GM-CSF,” Gastroenterology 151:710-723 (2016), which are hereby incorporated by reference in their entirety. Here, it is reported that the characterization of autoantibodies to GM-CSF in the serum of CD patients years before diagnosis, and propose that these antibodies contribute to disease development by disrupting the homeostatic role of GM-CSF via a complex signaling cross-talk between ILC3 and myeloid cells in the mucosa. First, it was demonstrated that the frequent and unique presence of IgG2 and IgA to GM-CSF is in a subset of CD patients, compared to UC, PAP, and HD, suggesting that these autoantibodies originate within the IgA plasma cell-rich gut mucosa. Anti-GM-CSF autoantibodies were not only associated with increased disease severity, complications and ileocolonic involvement in patients with active CD, much in line with previous reports, but remarkably, these autoantibodies were also predictive of severity, complications, and ileocolonic involvement at disease presentation up to 6 years before diagnosis in two independent cohorts. While total IgG antibodies against GM-CSF were highly enriched in CD patients, some reactivity of this isotype was seen in UC and HC. IgA antibodies reacting against GM-CSF, however, were an exclusive hallmark present in a group of CD patients and capable of blocking GM-CSFR signaling depending on posttranslational modifications on GM-CSF. The early detection of anti-GM-CSF autoantibodies, years before the diagnosis of CD, make this useful serological predictor and biomarker of complicated forms of ileocolonic CD, adding to the current gold standard serology (ASCA-IgA).

Despite an unaltered GM-CSFR expression and signaling capacity on myeloid cells in the CD mucosa, myeloid cells isolated from the INF CD mucosa displayed markedly reduced RA production in inflamed tissues. It was found that NCR⁺ILC3 were the major source of GM-CSF in the healthy NI CD mucosa, being reduced in numbers and GM-CSF output in the INF mucosa. Ex-RORγt NCR⁺ILC3/ILC1 produced higher levels of IFNγ. These observations demonstrate a lower GM-CSF output by tissue-resident sources (i.e. NCR⁺ILC3), affect homeostatic functions of myeloid cells. These results align well with previous reports identifying NCR⁺ILC3 as potent source of GM-CSF in healthy tissues, while reporting elevated numbers of inflammatory ex-RORγt NCR⁺ILC3/ILC1. Bernink et al., “Interleukin-12 and -23 Control Plasticity of CD127(+) Group 1 and Group 3 Innate Lymphoid Cells in the Intestinal Lamina Propria,” Immunity 43:146-160 (2015); Bernink et al., “Human Type 1 Innate Lymphoid Cells Accumulate in Inflamed Mucosal Tissues,” Nature Immunology 14:221-229 (2013); Croxatto et al., “Group 3 Innate Lymphoid Cells Regulate Neutrophil Migration and Function in Human Decidua,” Mucosal Immunology 9:1372-1383 (2016); Cella et al., “A Human Natural Killer Cell Subset Provides an Innate Source of IL-22 for Mucosal Immunity,” Nature 457:722-725 (2009); and Glatzer et al., “RORgammat(+) Innate Lymphoid Cells Acquire a Proinflammatory Program Upon Engagement of the Activating Receptor NKp44,” Immunity 38:1223-1235 (2013), all of which are hereby incorporated by reference in their entirety. Interestingly, ILC1s were recently reported to be associated the intestinal cellular immune signature of anti-TNF non-responder CD patients emphasizing underlining the importance of these findings. Martin et al., “Single-Cell Analysis of Crohn's Disease Lesions Identifies a Pathogenic Cellular Module Associated with Resistance to Anti-TNF Therapy,” Cell 178:1493-1508 (2019), which is hereby incorporated by reference in its entirety. While ILCs are believed to play a dispensable role during the anti-microbial defense in humans, due to the powerful proliferative capacity and dominating production of cytokines by T cells, their steady state function and role in maintaining mucosal tissue homeostasis remains widely appreciated. Rankin et al., “Complementarity and Redundancy of IL-22-producing Innate Lymphoid Cells,” Nature Immunology 17:179-186 (2016) and Vivier et al., “Innate Lymphoid Cells: 10 Years On,” Cell 174:1054-1066 (2018), which are hereby incorporated by reference in their entirety. Strikingly however is the functional plasticity of T cells, when compared to ILCs. Bedoui et al., “Parallels and Differences Between Innate and Adaptive Lymphocytes,” Nature. Immunology 17:490-494 (2016), which is hereby incorporated by reference in its entirety. T cell differentiation into Th1 or Th17 cells shows several emerging intermediate hybrid subsets (Th1/17), characterized by the expression of multiple synergistically acting cytokines (IFNy, TNFa and GM-CSF). Harbour et al., “Th17 Cells Give Rise to Th1 Cells that are Required for the Pathogenesis of Colitis,” Proceedings of the National Academy of Sciences of the United States of America 112:7061-7066 (2015), which is hereby incorporated by reference in its entirety. These “multi-cytokine-producer” are potent driver of autoimmune inflammation. Harbour et al., “Th17 Cells Give Rise to Th1 Cells that are Required for the Pathogenesis of Colitis,” Proceedings of the National Academy of Sciences of the United States of America 112:7061-7066 (2015), which is hereby incorporated by reference in its entirety. In contrast to this, ILC3s predominantly secrete homeostatic cytokines like IL-17, IL-22 or GM-CSF, implicating their role in supporting tissue homeostasis over inflammation. Mortha and Burrows, “Cytokine Networks Between Innate Lymphoid Cells and Myeloid Cells,” Frontiers in Immunology 9:191 (2018), which is hereby incorporated by reference in its entirety. However, synergistic actions of NCR⁺ILC3 and ex-RORγt NCR⁺ILC3/ILC1-secreted cytokines (e.g., GM-CSF and IFNy) could possibly generate a microenvironment that acts highly similar to cytokine-milieus generated by “multi-cytokine-producing” T cells. Mortha et al., “Microbiota-dependent Crosstalk Between Macrophages and ILC3 Promotes Intestinal Homeostasis,” Science 343:1249288 (2014); Mortha and Burrows, “Cytokine Networks Between Innate Lymphoid Cells and Myeloid Cells,” Frontiers in Immunology 9:191 (2018); and Lavin et al., “Regulation of Macrophage Development and Function in Peripheral Tissues,” Nature Reviews. Immunology 15:731-744 (2015), which are hereby incorporated by reference in their entirety. Both, synergizing ILC subsets and “multi-cytokine-producing” T cells may thus create a cytokine milieu fostering inflammation. The data of the present disclosure supports the idea of an intertwined local feed-back adaptation of ILC3 and myeloid cells. Mortha et al., “Microbiota-dependent Crosstalk Between Macrophages and ILC3 Promotes Intestinal Homeostasis,”

Science 343:1249288 (2014); and Bernink et al., “Interleukin-12 and -23 Control Plasticity of CD127(+) Group 1 and Group 3 Innate Lymphoid Cells in the Intestinal Lamina Propria,” Immunity 43:146-160 (2015), which are hereby incorporated by reference in their entirety. Plasma cells residing in the intestinal healthy mucosa, abrogating myeloid cell-derived RA production by neutralizing GM-CSF through anti-GM-CSF autoantibodies could thus favor the accumulation of inflammatory ex-RORγt NCR⁺ILC3/ILC1 by altering the NCR⁺ILC3 myeloid cell circuit (FIG. 11). CD pathogenesis is heterogenous and dependent on dietary components. Levine et al., “Evolving Role of Diet in the Pathogenesis and Treatment of Inflammatory Bowel Diseases,” Gut 67:1726-1738 (2018), which is hereby incorporated by reference in its entirety. Diminished intake of RA-precursors may favor a tissue immune state resulting in the accumulation of inflammatory ILCs within the intestinal mucosa. Spencer et al., “Adaptation of Innate Lymphoid Cells to a Micronutrient Deficiency Promotes Type 2 Barrier Immunity,”

Science 343:432-437 (2014); Goverse et al., “Vitamin A Controls the Presence of RORgamma+Innate Lymphoid Cells and Lymphoid Tissue in the Small Intestine,” Journal of Immunology 196:5148-5155 (2016); and Soares-Mota et al., “High Prevalence of Vitamin A Deficiency in Crohn's Disease Patients According to Serum Retinol Levels and the Relative Dose-Response Test,” World Journal of Gastroenterology 21:1614-1620 (2015), all of which are hereby incorporated by reference in their entirety. Anti-GM-CSF antibodies preceded the onset of CD by several years and may slowly, but with steadily increasing efficiency, alter anti-microbial defense, immune homeostasis and barrier integrity over the course of years. Nylund et al., “Granulocyte Macrophage-Colony-Stimulating Factor Autoantibodies and Increased Intestinal Permeability in Crohn Disease,”

Journal of Pediatric Gastroenterology and Nutricion 52:542-548 (2011) and Dabritz, “Granulocyte Macrophage Colony-Stimulating Factor and the Intestinal Innate Immune Cell Homeostasis in Crohn's Disease,” American Journal of Physiology. Gastrointestinal and Liver Physiology 306:G455-465 (2014), both of which are hereby incorporated by reference in their entirety. It is thus suggested that CD-associated anti-GM-CSF autoantibodies tip the intestinal immune tone towards inflammation and define a “pre-diseased” state of CD prior to the onset of disease by changing the local innate immune interactions. Intriguingly, the identification of posttranslational modifications on GM-CSF as epitopes for anti-GM-CSF autoantibodies, inspire the development of therapeutics with the potential to escape antibody-mediated neutralization. These agents could potentially reset the “pre-diseased” immune state or delay progression towards active disease in a subset of CD patients, prior to full manifestation of disease. Considering the outcome of previous clinical trials utilizing recombinant human GM-CSF for CD, a critical pre-selection of patients is advised prior to reevaluating GM-CSF-dependent therapeutic regimes and the design of personalized drug trials. These findings conclusively demonstrate a new mechanism for the development of a complicated form of CD in a larger subgroup of CD patients, allowing for the personalized classification of CD patients into anti-GM-CSF autoantibody positive and negative patients long before disease manifestation. Collectively these findings open new roads for a more precise diagnostic, classification and improved personalized treatment of CD patients.

### Example 9—Novel Variants of GM-CSF

The present disclosure relates to novel variants of GM-CSF that escape the neutralization of anti-GM-CSF autoantibodies detectable in the sera of Crohn's Disease (CD) patients up to 10 year prior to the onset of disease.

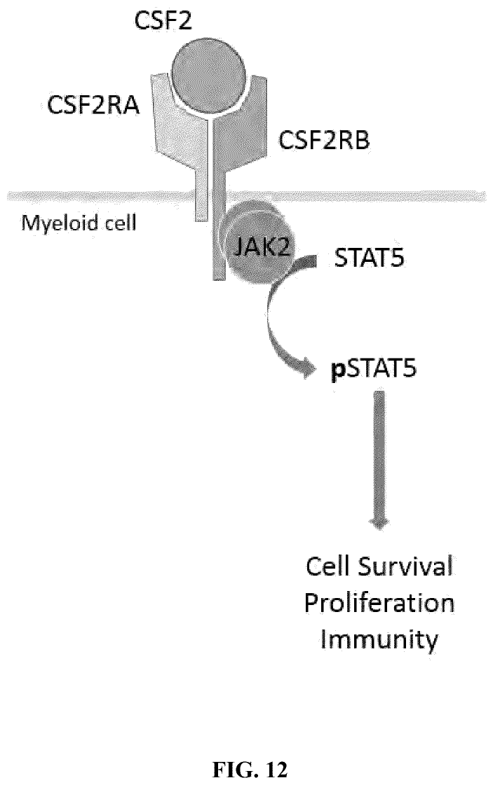

GM-CSF (i.e., CSF2) is important for the survival, differentiation, and function of mononuclear phagocytes (MNPs). GM-CSF signals via signal transducer and activator of transcription, STATS. Heterodimeric CSF2 receptor is expressed on myeloid subsets and signals through JAK2/STAT5 which supports anti-fungal/viral and bacterial defense and supports immune tolerance (Tregs/MDSC) (FIG. 12).

The findings described herein provide new protein variants of the myeloid growth and differentiation factor GM-CSF. Amino acid residues that are glycosylation sites were mutated to produce recombinant human GM-CSF in a human cell line. These variants are proposed to be unrecognizable by anti-GM-CSF autoantibodies found in the serum of CD patients. Highly sensitive ELISAs against GM-CSF will allow for the determination of whether a person will develop CD. This ELISA will further be useful to predict if a CD patient will develop a severe and complicated form of CD that often requires surgery.

Development of a multiplexable bead-based mass cytometry assay that will allow for the determination of (1) the isotype spectrum of anti-GM-CSF autoantibodies in multiple serum samples at the same time and (2) reveal the recombinant GM-CSF variants these autoantibodies react against to ultimately identify heterogeneity in CD patients presenting with anti-GM-CSF autoantibodies.

Collectively, this disclosure provides a precision diagnostic assay and personalized therapeutic for the improved detection and categorization of CD patients prone to develop a severe and complicated form of CD.

A synthesized cDNA encoding for human GM-CSF with and C-terminal Enterokinase-site followed by a 6 His-tag has been cloned into the eukaryotic expression vector pIRESpuro. Q5 site directed mutagenesis has been used to generate mutations S22A, S24A, S26A, T27A, N44A, N54A. Variations carrying individually mutated amino acids have been used to generate variants carrying two, three, four, five or all six sites mutated to Alanine. HEK293 cells have been transiently transfected with pIRESpuro containing one of these variants for the generation of clone stably integrating the recombinant DNA into their genome. Newly generated clones are expanded and GM-CSF production is validated using ELISA and intracellular antibody staining and analysis by flow cytometry. Bioactivity of the produced variants in test on U937 cell. U937 cells are stimulated with cell culture supernatant from HEK293 cells expressing GM-CSF variants. STAT5 phosphorylation is evaluated using phospho STAT5 flow. Cell culture supernatant of HEK293 cells is collected and 6x His-tag carrying GM-CSF is purified using Nickle columns. GM-CSF containing eluates are enriched using size exclusion columns. Recovered GM-CSF is then tested for molecular weight and glycosylation using SDS-PAGE and anti-GM-CSF Western blot. Using this process up to 100ug of protein/20 ml of condition media is currently able to be enriched. Recombinant variants will alternatively be stable transfected into other human cell lines to compare the glycosylation pattern of GM-CSF derived from different cellular sources.

The generated variants will either be used to coat high-binding 96 well ELISA plates. Serum samples and polyclonal goat anti-human GM-CSF sera will be used to set up ELISAs that will allow for detection of reactivity against GM-CSF and its different glycosylation in sera from healthy individuals, CD or UC patients. Healthy subjects at familiar risk of developing CD will be tested for the presence of anti-GM-CSF autoantibodies. Using this method, individuals at risk of developing a complicated form of CD disease may be identified.

Genetically engineered recombinant human GM-CSF variants will be used, covalently coupled to latex beads of different sizes (2, 4, 6, 8, 10, 12, 14, 16, 18 p.m in diameter). Each bead of a given size will be coated with one genetically engineered GM-CSF variant. Beads will then be pooled into a tube at equal ratios and used in small reaction volumina of 20-50 μl of serum from anti-GM-CSF positive Crohn's Disease patients. Following the incubation with sera, bead and GM-CSF variants bound by anti-GM-CSF autoantibodies will be stained with anti-human IgA, anti-human IgM and anti-human IgG or total anti-human Ig secondary antibodies. These secondary antibodies can either be coupled to distinct metal isotopes (for the use in mass cytometry) or fluorophores (for the use in flow cytometry). After washing the beads, samples will be analyzed on a flow or mass cytometer, revealing anti-GM-CSF autoantibody staining on beads of specific size, reflecting the specific epitope/epitopes of the serum and signals/fluorescence for specific isotypes, to reveal the heterogeneity in antibody isotypes reacting against specific epitopes on GM-CSF.

IBD-associated anti-GM-CSF autoantibodies are distinct and alter GM-CSFR signaling. Anti-GM-CSF autoantibodies identify severe forms of CD as described herein.

CD-associated anti-GM-CSF autoantibodies block GM-CSFR signaling. It is also discovered herein that anti-GM-CSF autoantibodies recognize structural epitopes. Post-translational modified GM-CSF is targeted by anti-GM-CSF autoantibodies in CD. Moreover, anti-GM-CSF autoantibodies are a predictive biomarker for Crohn's Disease. Anti-CSF2 titers are elevated in a subgroup of CD patients which is correlated with Ileal CD and increased disease severity. Anti-CSF2 Abs have CD-specific isotypes which precede the onset of active disease by years and recognize post-translational modifications (PTM). Removal of PTM rescues effect of anti-CSF2 Abs. Genetic engineering of CSF2 was achieved to avoid recognition by anti-CSF2 Abs for a personalized therapy of CD. Production of recombinant CSF2 designed to lack specific glycosylation sites. PTM-specific anti-CSF2 ELISA as diagnostic assay for the subclassification of CD patients. Use of recombinant CSF2 variants as therapeutic for sero-positive anti-CSF2 patients.

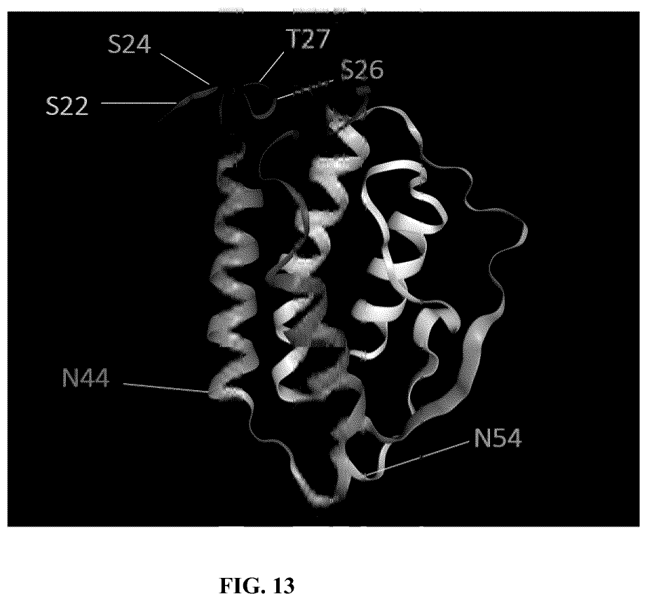

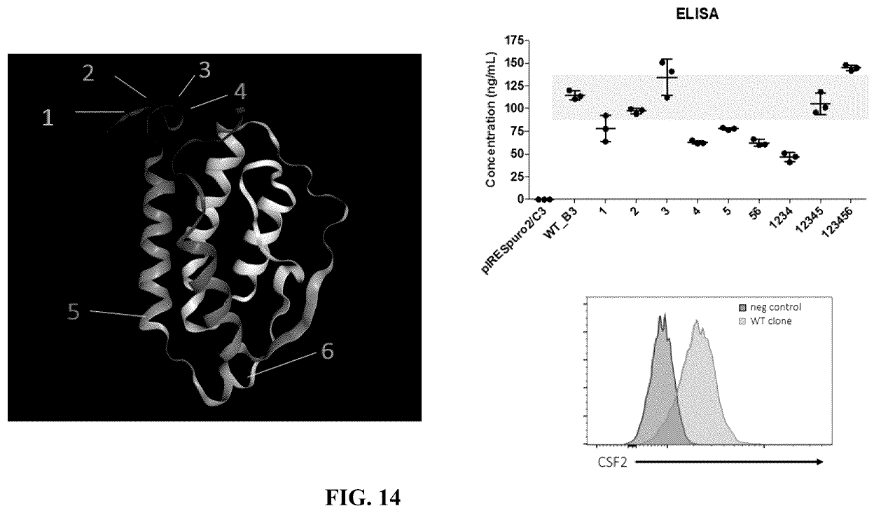

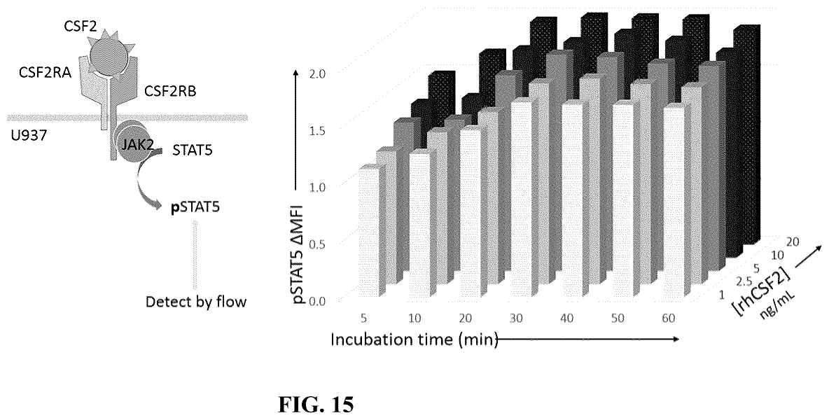

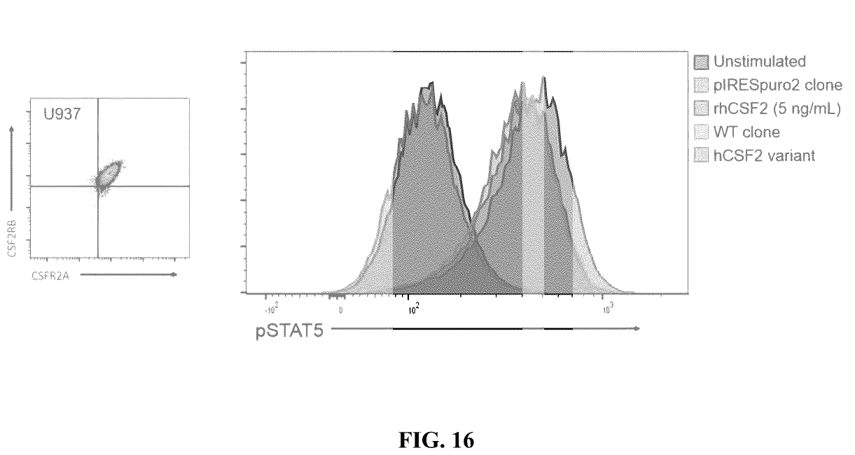

Glycosylation sites on human CSF2, for example, include S22, S24, T27, S26, N44, and/or N54, as shown in FIG. 13. Glycosylation in accordance with the present disclosure is important for protein structure, function, and stability (half-life). In one example as shown in FIG. 14, stable cell lines expressing human CSF2 deficient in glycosylation sites are produced. STATS phosphorylation in accordance with the present disclosure may be stimulated in U937 cells with recombinant human CSF2 (FIG. 15). As shown in FIG. 16, recombinant human CSF2 deficient in glycosylation sites is biologically active.

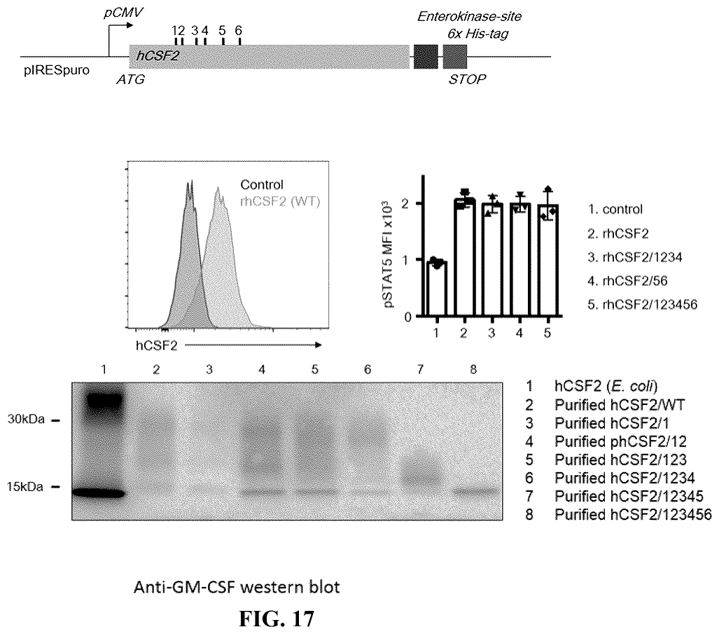

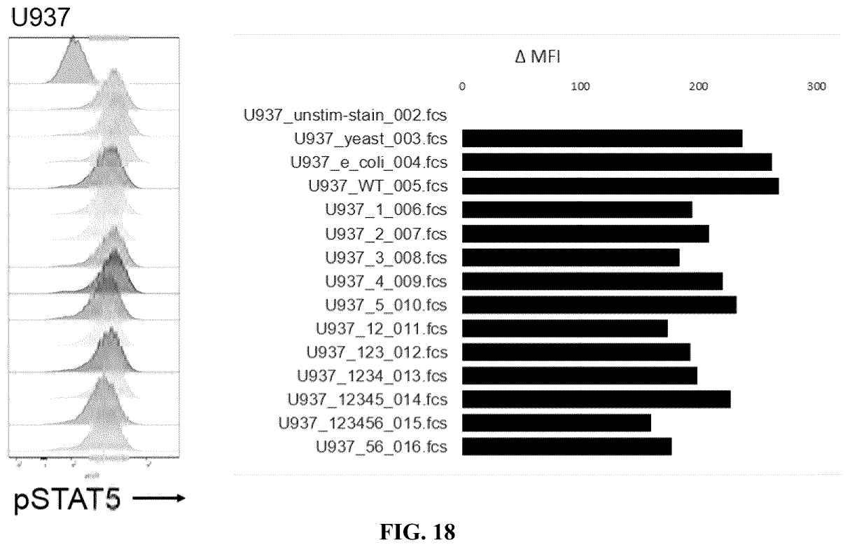

In one embodiment, HIS-tag purification yields recombinant human CSF2 from stable HEK293 clones lacking one or all glycosylations (FIG. 17). Purification of recombinant human CSF2 deficient in glycosylation does not alter biologically activity (FIG. 18). Patient serum contains anti-GM-CSF autoantibodies against wild type variant of GM-CSF, specifically recognizing Variants 2 and 4 with anti-IgA antibodies against the wild type form and IgM and IgG antibodies recognizing the GM-CSF variants. Variants recognized by the serum will then be considered as potential intervention therapeutic.

The generation of human GM-CSF variants that lack either one individual, or all possible combinations of posttranslational glycosylations. An ELISA that uses these GM-CSF variants identifies CD patients. The use of the GM-CSF variants in mass or flow cytometry multiplexable bead-based assays to simultaneously identify the epitopes and isotypes of anti— GM-CSF autoantibodies in sera of CD patients.

### Example 10—GM-CSF Is Abnormally Glycosylated in Crohn's Disease

The glycosylation profile of GM-CSF protein was evaluated. The recombinant GM-CSF from two different origins, yeast and CHO-produced cells was used. On this regard, it is important to notice that the N-glycosylation machinery from yeast is different (less complex) from the mammalians, which raise questions about its translational to human GM-CSF patients. For the characterization of the glycosylation profile of GM-CSF, a lectin blot was performed, using L-PHA (that recognizes β1,6-GlcNAc branched N-glycans), MALII (that recognizes α2,3-sialic acid), GNA (recognizing high-mannose N-glycans) and AAL (that recognizes core fucose structures) (FIG. 20A). Interestingly, GM-CSF produced in mammalian cells displays a higher molecular weight isoform, which indicates that GM-CSF is post-translationally modified with a significant % of glycans structures. This isoform has shown to display a positive reactivity to L-PHA and AAL lectins (FIG. 20B). This specific glycosignature suggests that this isoform is modified with complex branched and fucosylated N-glycans structures. The lower band of CHO-producing GM-CSF has positive reactivity to MALII and GNA, revealing the presence of a potential hybrid N-glycan structure with terminal sialylation (FIG. 20B). Concerning, yeast-producing GM-CSF, it only displays high- mannose N-glycans (FIG. 20B), which are typically found in lower organisms as fungi.

Overall, GM-CSF from mammalian cells was demonstrated to have two glycoforms, in which the heavier is modified by complex branched and fucosylated N-glycans and the lower glycoform could potentially be a hybrid N-glycan with terminal sialylation. These results demonstrate that indeed GM-CSF is significantly modified with complex branched N-glycans structures, highlighting the importance of the glycosylation of GM-CSF in the modulation of its functions both in health and disease.

Additionally, and to gain further insights on whether and how GM-CSF is abnormally glycosylated in CD and consequently associated with disease pathogenesis, serum from CD patients and healthy donors (HD) was isolated and the glycoprofile of GM-CSF was characterized. A lectin ELISA was performed, in which serum GM-CSF from CD and HD were captured and incubated with L-PHA, GNA and AAL lectins. The results showed no differences in the levels of L-PHA binding, however, GNA binding was significantly increased in GM-CSF from CD patients, suggesting that in CD patients GM-CSF has an increased expression of mannosylated N-glycans (FIG. 20C). In contrast, AAL binding was significantly reduced in GM-CSF from CD patients, suggesting a decreased presence of core fucose residues (FIG. 20C). Levels of GM-CSF are similar between CD and HD. Overall, the increased high-mannose structures and decrease in the core- fucose revealed a specific glycosignature of GM-CSF in CD patients when comparing with HD.

### Example 11—Materials and Methods for Example 10

Lectin Blot and Western Blot—The glycoprofile of recombinant forms of GM-CSF was evaluated by lectin blot. 2 μg of purified protein from yeast and CHO cells were subjected to 15% SDS-PAGE electrophoresis and membranes were blocked with BSA 4% before incubation with lectins Phaseolus Vulgaris Leucoagglutinin (L-PHA), Maackia Amurensis Lectin II (MAL-II), Galanthus Nivalis Lectin (GNA) and Aleuria Aurantia Lectin (AAL) (Vector Labs; 2 ug/mL). Bands were then visualized using the Vectorstain Elite ABC kit (Vector Labs) and the detection was performed using ECL reagent (GE Healthcare, Life Sciences).

For western blot analysis with anti-GM-CSF, 51 μg of purified protein from yeast and CHO cells were used. The samples were subjected to 15% SDS-PAGE electrophoresis and membranes were blocked with milk before incubation with biotinylated GM-CSF antibody (0.1 μg/mL R&D Systems). The target protein was then visualized using the Vectorstain Elite ABC kit (Vector Labs) and the detection was performed using ECL reagent (GE Healthcare, Life Sciences).

Serum collection—Blood samples from four adult Crohn's disease (CD) patients and two pediatric CD patients were collected at Centro Hospitalar Universitário do Porto (CHUP) and Hospital de São João (HSJ), respectively. All patients were diagnosed with active CD and were naïve for treatment with biologics at the time of blood collection. Blood samples from healthy donors were used as controls. All participants gave informed consent about all clinical procedures and research protocols were approved by the ethical committee of both hospitals. Serum was collected from peripheral blood by centrifugation at 1620× g for 10 minutes and stored at−-80oC until analysis.

Lectin ELISA—Detection of lectin-binding motifs in the serum GM-CSF from CD patients and HD was performed by a lectin ELISA adapted from Astrom et al., “Reverse Lectin ELISA for Detecting Fucosylated Forms of α1-acid Glycoprotein Associated With Hepatocellular Carcinoma,” PlosOne (2017), which is hereby incorporated by reference in its entirety. Briefly, Microtiter plates (Maxisorp, Nunc) were coated with a mouse anti-human GM-CSF (DuoSet, R&DSystems) in PBS buffer overnight at room temperature (RT). Blocking was performed with Carbo-free blocking solution (Vector Labs) for 1 h at RT. Plates were washed 5 times with PBS+0.05% Tween 20 (PBST) before CD/HD plasma samples, diluted 1:50 in PBS containing 1% Carbo-free blocking solution (diluent solution) (Vector Labs), were added and incubated shaking at 200 rpm for 2h at RT. After washing as described above, biotinylated lectins, diluted 1:1000 in diluent solution (Vector Labs), were added and incubated for 1 h shaking at 200 rpm at RT. Bound lectin was detected using an HRP-conjugated streptavidin (DuoSet R&D Systems) incubated for 20 minutes and Tetramethylbenzidine substrate (DuoSet R&D Systems) was incubated for 20 minutes protected from dark. Reaction was stopped using H2SO4 and the amount of bound lectin was measured at 450 nm using a μQuant Microplate Reader (BioTek, Agilent).

GM-CSF ELISA—Prior to serum capture, serum from CD patients and HD was concentrated using Amicon® Ultra-2 mL Centrifugal Filters, to reach a final concentration of 4X. Microtiter plates (Maxisorp, Nunc) were coated with a mouse anti-human GM-CSF (DuoSet R&D Systems) in PBS buffer, overnight at room temperature (RT). Blocking was performed with Carbo-free blocking solution (Vector Labs) for 1 h at RT. Plates were washed 5 times with PBS+0.05% Tween 20 (PB ST) before the CD/HD plasma samples in PBS containing 1% Carbo-free blocking solution (diluent solution) were added and incubated shaking at 200 rpm for 2 h at RT. After washing the wells as described above, biotinylated mouse anti-human GM-CSF (DuoSet R&D Systems) was added and incubated for 2 h shaking at 200 rpm at RT. Bound GM-CSF was detected using an HRP-conjugated streptavidin (DuoSet R&D Systems) incubated for 20 minutes and Tetramethylbenzidine substrate (DuoSet R&D Systems) was incubated for 20 minutes protected from dark. Reaction was stopped using H2504 and the amount of bound lectin was measured at 450 nm using a 1.tQuant Microplate Reader (BioTek, Agilent).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1-69. (canceled)

70. A method of preventing or treating one or both of an inflammatory bowel disease (IBD) and a condition resulting from the IBD in a subject comprising:
selecting a subject having or at risk of having the IBD and
administering a Granulocyte Macrophage-Colony Stimulating Factor (GM-CSF) protein to the selected subject under conditions effective to prevent or treat one or both of the IBD and a condition resulting from the IBD in the subject.

71. The method of claim 70, wherein the GM-CSF protein is glycosylated.

72. The method of claim 70, wherein the GM-CSF protein is not glycosylated.

73. The method of claim 70, wherein the GM-CSF comprises an amino acid substitution at S22.

74. The method of claim 70, wherein the GM-CSF comprises an amino acid substitution at S24.

75. The method of claim 70, wherein the GM-CSF comprises an amino acid substitution at T27.

76. The method of claim 70, wherein the GM-CSF comprises an amino acid substitution at S26.

77. The method of claim 70, wherein the GM-CSF comprises an amino acid substitution at N44.

78. The method of claim 70, wherein the GM-CSF comprises an amino acid substitution at N54.

79. The method of claim 70, wherein the GM-CSF comprises an alanine substitution at one or more of S22, S24, T27, S26, N44, and N54.

80. The method of claim 70, wherein the administering comprises inhalation, intranasal instillation, topically, transdermally, intradermally, parenterally, subcutaneously, intravenous injection, intra-arterial injection, intramuscular injection, intrapleurally, intraperitoneally, intrathecally, or application to a mucous membrane.

81. The method of claim 70, wherein the administering comprises administering a pharmaceutical composition, wherein the pharmaceutical composition comprises the GM-CSF and a pharmaceutically acceptable carrier.

82. The method of claim 70, wherein the IBD is Crohn's disease.

83. The method of claim 70, wherein the IBD is ulcerative colitis.

84. The method of claim 70, further comprising administering to the subject one or more additional agent that prevents or treats the IBD and/or a condition resulting from the IBD, wherein the additional agent is selected from an antibiotic, an anti-inflammatory, or an immunosuppressant.

85. The method of claim 70, wherein the selecting comprises detecting or having detected the presence of anti-GM-CSF autoantibodies in a sample from the subject.

86. The method of claim 85, wherein the sample is selected from whole blood, serum, urine, and nasal excretion.

87. The method of claim 85, wherein the GM-CSF comprises an amino acid substitution at one or more of S22, S24, T27, S26, N44, and N54.

88. The method of claim 85, wherein the administering comprises administering a pharmaceutical composition, wherein the pharmaceutical composition comprises the GM-CSF and a pharmaceutically acceptable carrier.

89. The method of claim 70, wherein the IBD is Crohn's disease or ulcerative colitis.
